SEQUENCE LISTING (136 sequences)

SEQ ID NO: 1 - protein, 207 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSS

SEQ ID NO: 2 - protein, 225 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQY

SEQ ID NO: 3 - protein, 231 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSK

SEQ ID NO: 4 - protein, 233 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTI

SEQ ID NO: 5 - protein, 374 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 6 - protein, 232 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNGGSVPADPPTAFNKDKLNSFITQYSTGQVSVEIEWELQKEG

SEQ ID NO: 7 - protein, 472 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYENLYFQGASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQY

SEQ ID NO: 8 - protein, 230 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSR

SEQ ID NO: 9 - protein, 374 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 10 - protein, 317 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLGGSGGGSKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 352 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLGGSGGGSVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 361 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGSDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 370 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 285 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLVAGPSNMAVQGRNYSKRWNPEIQYTSNYYKSNNVEFAVNTEGVYSEPRPIGTRYLTRNL

SEQ ID NO: 15 - protein, 368 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGSGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 294 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 518 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 513 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSGGSEFAVNTEGVYSEPRPIGTRYLTRNL

SEQ ID NO: 19 - protein, 507 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSGGSEFAVNTEGVYSEPRPIGTRYLTRNL

SEQ ID NO: 20 - protein, 495 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSGGTRYLTRNL

SEQ ID NO: 21 - protein, 493 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNV

SEQ ID NO: 22 - protein, 1035 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGENLYFQSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNV

SEQ ID NO: 23 - protein, 234 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGGSQSLDRLMNPLIDQYLYYLSR

SEQ ID NO: 24 - protein, 378 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGGSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 25 - protein, 229 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGGSQSLDRLMNPLIDQY

SEQ ID NO: 26 - protein, 378 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 27 - protein, 235 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGSGGGSQSLDRLMNPLIDQYLYYLSR

SEQ ID NO: 28 - protein, 379 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGSGGGSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 29 - protein, 230 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGSGGGSQSLDRLMNPLIDQY

SEQ ID NO: 30 - protein, 379 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGSGGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 31 - protein, 236 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGSQSLDRLMNPLIDQYLYYLSR

SEQ ID NO: 32 - protein, 380 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 33 - protein, 231 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGSQSLDRLMNPLIDQY

SEQ ID NO: 34 - protein, 380 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 35 - protein, 237 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGGSQSLDRLMNPLIDQYLYYLSR

SEQ ID NO: 36 - protein, 381 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGGSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 37 - protein, 232 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGGSQSLDRLMNPLIDQY

SEQ ID NO: 38 - protein, 381 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 39 - protein, 238 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGGGSQSLDRLMNPLIDQYLYYLSR

SEQ ID NO: 40 - protein, 382 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGGSGGGGSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAA

SEQ ID NO: 41 - protein, 233 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGGGSQSLDRLMNPLIDQY

SEQ ID NO: 42 - protein, 382 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSGGGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQ

SEQ ID NO: 43 - protein, 212 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSGGGSG

SEQ ID NO: 44 - protein, 505 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGSDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGM

SEQ ID NO: 45 - protein, 509 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGSGGGSDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGM

SEQ ID NO: 46 - protein, 367 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGSGGGSQSLDRLMNPLIDQYLGGGGSGGGSDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 364 aa, synthetic construct
GVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHGGSGGGSQSLDRLMNPLIDQYLGGSGGGSDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 48 - protein, 287 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGGSGGGGSKHPPPQILIKNGSQYSTGQVSVEIEWELQKENSKRWNPEIQYTSNY

SEQ ID NO: 49 - protein, 300 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGGSGGGGSKHPPPQILIKNTPVPADPGSLNSFITQYSTGQVSVEIEWELQKENSKRWNPEIQYTSNY

SEQ ID NO: 50 - protein, 337 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGSGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNY

SEQ ID NO: 51 - protein, 336 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGSGGGSQSLDRLMNPLIDQYLGGGGSGGGSDVYLQGPIWAKIPHTDGNFHPSPLMGGFGMKHPPPQILIKNTPVPADPPTAFNKDKLNSFITQYSTGQVSVEIEWELQKENSKRWNPEIQYTSNY

SEQ ID NO: 52 - protein, 304 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGSGGGSGGSKHPPPQILIKNTPVPADPGSLNSFITQYSTGQVSVEIEWELQKENSKRWNPEIQYTSGGTRYLTRNL

SEQ ID NO: 53 - protein, 332 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGSDVYLQGPIWAKIPHTDGNFHPSPLMGGFGMKHPPPQILIKNTPVPADPGSLNSFITQYSTGQVSVEIEWELQKENSKRWNPEIQYTSGGTRYLTRNL

SEQ ID NO: 54 - protein, 268 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGSGGSGGSGGSKHPPPQILIKNGGSTQYSTGQVSVEIEWELQKEN

SEQ ID NO: 55 - protein, 274 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGSGGSGGSGGSKHPPPQILIKNGGSTQYSTGQVSVEIEWELQKEN

SEQ ID NO: 56 - protein, 266 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGSGGSGGSGGSKHPPPQILIKNGSQYSTGQVSVEIEWELQKEN

SEQ ID NO: 57 - protein, 272 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGSGGSGGSGGSKHPPPQILIKNGSQYSTGQVSVEIEWELQKEN

SEQ ID NO: 58 - protein, 269 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGGGSGGGGSGGGGSKHPPPQILIKNGSQYSTGQVSVEIEWELQKEN

SEQ ID NO: 59 - protein, 275 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNGSQYSTGQVSVEIEWELQKEN

SEQ ID NO: 60 - protein, 289 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNGSQYSTGQVSVEIEWELQKENSKRWNPEIQYTSNY

SEQ ID NO: 61 - protein, 297 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNGSQYSTGQVSVEIEWELQKENSKRWNPEIQYTSGGTRYLTRNL

SEQ ID NO: 62 - protein, 282 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGSGGGSGGGSKHPPPQILIKNTPVPADGSNSFITQYSTGQVSVEIEWELQKENS

SEQ ID NO: 63 - protein, 295 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSSGGSGGSLCNTRNQSLDRLMNPLIDQYLGGGSGGGSGGGSKHPPPQILIKNTPVPADGSNSFITQYSTGQVSVEIEWELQKENS

SEQ ID NO: 64 - protein, 283 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGGGSGGGGSGGGGSKHPPPQILIKNTPVPADPGSLNSFITQYSTGQVSVEIEWELQKENS

SEQ ID NO: 65 - protein, 289 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNTPVPADPGSLNSFITQYSTGQVSVEIEWELQKENS

SEQ ID NO: 66 - protein, 302 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNTPVPADPGSLNSFITQYSTGQVSVEIEWELQKENSKRWNPEIQYTSNY

SEQ ID NO: 67 - protein, 310 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGGGSGGGGSGGGGSKHPPPQILIKNTPVPADPGSLNSFITQYSTGQVSVEIEWELQKENSKRWNPEIQYTSGGTRYLTRNL

SEQ ID NO: 68 - protein, 291 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGSGGSGGSGGSKHPPPQILIKNTPVPADPGSLNSFITQYSTGQVSVEIEWELQKENSKRWNPEIQYTS

SEQ ID NO: 69 - protein, 297 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGSGGSGGSGGSKHPPPQILIKNTPVPADPGSLNSFITQYSTGQVSVEIEWELQKENSKRWNPEIQYTS

SEQ ID NO: 70 - protein, 300 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGSGGSGGSGGSKHPPPQILIKNTPVPADPGSLNSFITQYSTGQVSVEIEWELQKENSKRWNPEIQYTSGTRYLTRNL

SEQ ID NO: 71 - protein, 306 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGSGGSGGSGGSKHPPPQILIKNTPVPADPGSLNSFITQYSTGQVSVEIEWELQKENSKRWNPEIQYTSGTRYLTRNL

SEQ ID NO: 72 - protein, 301 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIGGSGGSGGSGGSKHPPPQILIKNTPVPADPGSLNSFITQYSTGQVSVEIEWELQKENSKRWNPEIQYTSGGTRYLTRNL

SEQ ID NO: 73 - protein, 307 aa, synthetic construct
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSLCNTRNMNPLIGGSGGSGGSGGSKHPPPQILIKNTPVPADPGSLNSFITQYSTGQVSVEIEWELQKENSKRWNPEIQYTSGGTRYLTRNL

SEQ ID NO: 74 - protein, 579 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGGSGGGGDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 75 - protein, 577 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGSGGGDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 76 - protein, 584 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGGGSGGGGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 77 - protein, 579 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGGGSGGGGDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 78 - protein, 548 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGGGSGGGGDVYLQGPIWAKIPHTDGHFHPSPLMGGFGLKHPPPQILIKNTPVPADPPTTFNQSKLNSFITQYSTGQVSVEIEWELQKENSKRWNPEIQYTSNY

SEQ ID NO: 79 - protein, 587 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 80 - protein, 593 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 81 - protein, 590 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 82 - protein, 596 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 83 - protein, 593 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGSGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 84 - protein, 599 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLGGSGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 85 - protein, 596 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGSGGSGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 86 - protein, 602 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLGGSGGSGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 87 - protein, 600 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLEFAWPGQQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - protein, 593 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSQSLDRLMNPLIDQYLGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 610 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - protein, 564 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLDVYLQGPIWAKIPHTDGNFHPSPLMGGFGMKHPPPQILIKNTPVPADPPTAFNKDKLNSFITQYSTGQVSVEIEWELQKENSKRWNPEIQYTGGVEFAVNTEGVYSEPRPIGTRYLTRNL

SEQ ID NO: 91 - protein, 606 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTITNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 92 - protein, 603 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 93 - protein, 596 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSQSLDRLMNPLIDQYLGGSGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 94 - protein, 707 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSGGSGGSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVGGSGGSGGSKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 594 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 96 - protein, 613 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIEFAWPGQTNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 97 - protein, 584 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTGGVDFAVNTEGVYSEPRPIGTRYLTRNL

SEQ ID NO: 98 - protein, 587 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTGGVDFAVNTEGVYSEPRPIGTRYLTRNL

SEQ ID NO: 99 - protein, 581 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTGGVDFAVNTEGVYSEPRPIGTRYLTRNL

SEQ ID NO: 100 - protein, 590 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLGGSGGSGGSADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTGGVDFAVNTEGVYSEPRPIGTRYLTRNL

SEQ ID NO: 101 - protein, 700 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWGGSGGSGGSKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 102 - protein, 590 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLGGGSGGGGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 103 - protein, 583 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSGGSGGSQSLDRLMNPLIDQYLGGGSGGGDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 104 - protein, 532 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAGGGGSGGGGKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 516 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFGSGKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 106 - protein, 561 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHGGGSQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 107 - protein, 1247 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGENLYFQGASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNV

SEQ ID NO: 108 - protein, 587 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 109 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 110 - DNA, 8686 bp, synthetic construct
  source 1..8686: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcggtcaccagccaacaatgtgcggatggcgttacgacttactgggcctgatctcaccgctttagataccgcacactgggcaatacgaggtaaagccagtcgaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 111 - DNA, 8936 bp, synthetic construct
  source 1..8936: mol_type = other DNA
ttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggcgcgcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactgaattcacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctt

SEQ ID NO: 112 - protein, 483 aa, synthetic construct
GGGAPMADNNEGADGVGNSSGNWHCDSTWLGDRVITTSTRTWALPTYNNHLYKQISSGNDNYFGYSTPWGYFDFNRFHCHFSPRDWQRLINNNWGFRPKRLFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFFSYTFEDVPFHSSYAHSQSLDRLMNPLIDQYLYYLSRTQGTLLFSQAGPMSQAKNWLPGPCYRQQRVSTNNNSNFWTGATKYHLNGRDSLVNPGAMATHKDDEEFFPSGVLIFGKQGAGNDVMTEEEIKTTNPVATEQYGVANLQNTPTGVNQGALPGMVWQNRDVYLQGPIWAKIPHTDGHFHPSPLMGGFGLKHPPPQILIKNTPVPANPPTTFSAKASFITQYSTGQVSVEIEWELQKENSKRWNPEIQYTSNYKSNVDFAVDTGVYSEPRPIGTRYLTRNL

SEQ ID NO: 113 - protein, 531 aa, synthetic construct
GGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - protein, 532 aa, synthetic construct
GGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 115 - protein, 4 aa, synthetic construct
SGGG

SEQ ID NO: 116 - protein, 4 aa, synthetic construct
GGGS

SEQ ID NO: 117 - protein, 5 aa, synthetic construct
GSGGG

SEQ ID NO: 118 - protein, 6 aa, synthetic construct
GGGSGG

SEQ ID NO: 119 - protein, 6 aa, synthetic construct
GGSGGG

SEQ ID NO: 120 - protein, 6 aa, synthetic construct
SGGSGG

SEQ ID NO: 121 - protein, 6 aa, synthetic construct
GGSGGS

SEQ ID NO: 122 - protein, 7 aa, synthetic construct
GGSGGGS

SEQ ID NO: 123 - protein, 7 aa, synthetic construct
GGGSGGG

SEQ ID NO: 124 - protein, 7 aa, synthetic construct
GGSGGSG

SEQ ID NO: 125 - protein, 8 aa, synthetic construct
GGGSGGGG

SEQ ID NO: 126 - protein, 9 aa, synthetic construct
GGGGSGGGS

SEQ ID NO: 127 - protein, 9 aa, synthetic construct
GGGGSGGGG

SEQ ID NO: 128 - protein, 9 aa, synthetic construct
GGSGGSGGS

SEQ ID NO: 129 - protein, 11 aa, synthetic construct
GGGSGGGSGGS

SEQ ID NO: 130 - protein, 12 aa, synthetic construct
GGSGGSGGSGGS

SEQ ID NO: 131 - protein, 15 aa, synthetic construct
GGGGSGGGGSGGGGS

SEQ ID NO: 132 - protein, 12 aa, synthetic construct
GGSGGSLCNTRN

SEQ ID NO: 133 - protein, 9 aa, synthetic construct
GGENLYFQS

SEQ ID NO: 134 - protein, 7 aa, synthetic construct
ENLYFQG

SEQ ID NO: 135 - protein, 9 aa, synthetic construct
GGENLYFQG

SEQ ID NO: 136 - protein, 7 aa, synthetic construct
GGGSGGS

(19) United States
(12) Patent Application Publication
(10) Pub. No.: US 2023/0086710 A1
(43) Pub. Date: Mar. 23, 2023

(54) AAV CAPSID VARIANTS

(71) Applicant: California Institute of Technology, Pasadena, CA (US)

(72) Inventors: Xiaozhe Ding, Pasadena, CA (US); Viviana Gradinaru, La Canada Flintridge, CA (US)

(21) Appl. No.: 17/931,808

(22) Filed: Sep. 13, 2022

Related U.S. Application Data
(60) Provisional application No. 63/244,132, filed on Sep. 14, 2021.

(51) Int. Cl. C07K 14/075; C12N 15/86

(52) U.S. Cl. CPC C07K 14/075; C12N 15/86; C12N 2750/14122

(57) ABSTRACT

Disclosed herein include methods and compositions comprising variant AAV capsids. Variant capsid proteins, including variant capsid proteins with structure-guided deletion/substitution, tandem multimers, and/or variant capsid proteins with structure-guided deletion and C-terminal insertion, are provided in some embodiments. The variant capsid proteins disclosed herein are capable of assembling into a variant AAV capsid with an expanded size (e.g., diameter) and/or genetic cargo capacity. Methods of treating diseases and disorders using said rAAV are also disclosed.

### RELATED APPLICATIONS

This application claims the benefit under 35 U.S.C. § 119(e) to U.S. Provisional application No. 63/244,132, filed Sep. 14, 2021, the content of this related application is incorporated herein by reference in its entirety for all purposes.

### STATEMENT REGARDING FEDERALLY SPONSORED R&D

This invention was made with government support under Grant No. NS111369 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302452-WO_Sequence listing.xml, created Sep. 13, 2022, which is 180 kilobytes in size. The information in the electronic format of the Sequence Listing is incorporated herein by reference in its entirety.

### BACKGROUND

### Field

The present disclosure relates generally to the field of gene delivery. More particularly, the application relates to engineered adeno-associated viruses (AAV) of improved cargo-delivery capability, including an expanded size capable of packaging oversized cargoes.

### Description of the Related Art

The AAV capsid, a 25-nm protein nanoparticle, has been widely applied as an in vivo gene delivery vector because of its low immunogenicity, engineerable tropism, and excellent safety profile. However, the capsid's small physical volume imposes a modest upper limit of ˜5 kb on its cargo capacity. Oversized cargoes are truncated to fit this limit when packaged. This restriction precludes single-vector packaging of many important genetic cargoes, including a number of CRISPR-based tools, many cell-type-specific promoters and enhancers, and at least 6% of human cDNAs including many disease-related genes. Because the icosahedral capsid has an evolutionarily conserved geometry, altering the size of the AAV capsid has been challenging. Thus, despite the demand for a larger-sized AAV capsid, there is a lack of success in altering the size of AAV capsids. This can be due to its evolutionarily conserved geometry. Accordingly, there is a need for AAV capsids of expanded size to enable larger genetic cargos and improved cargo-delivery capabilities.

### SUMMARY

Disclosed herein include variant adeno-associated virus (AAV) capsid proteins. In some embodiments, the variant AAV capsid protein comprises: (a) fifty or more of amino acid residues functionally equivalent to amino acids 452 to 577 of the AAV9 VP1 protein (SEQ ID NO: 109) have been deleted or substituted, (b) twenty or more of amino acid residues functionally equivalent to amino acids 597 to 640 of the AAV9 VP1 protein (SEQ ID NO: 109) have been deleted or substituted, (c) five or more of amino acid residues functionally equivalent to amino acids 656 to 669 of the AAV9 VP1 protein (SEQ ID NO: 109) have been deleted or substituted, (d) five or more of amino acid residues functionally equivalent to amino acids 692 to 736 of the AAV9 VP1 protein (SEQ ID NO: 109) have been deleted or substituted, or a combination thereof.

In some embodiments, one hundred or more of amino acid residues functionally equivalent to amino acids 452 to 577 of the AAV9 VP1 protein (SEQ ID NO: 109) have been deleted or substituted, (b) thirty or more of amino acid residues functionally equivalent to amino acids 596 to 640 of the AAV9 VP1 protein (SEQ ID NO: 109) have been deleted or substituted, (c) ten or more of amino acid residues functionally equivalent to amino acids 656 to 669 of the AAV9 VP1 protein (SEQ ID NO: 109) have been deleted or substituted, (d) twenty or more of amino acid residues functionally equivalent to amino acids 692 to 736 of the AAV9 VP1 protein (SEQ ID NO: 109) have been deleted or substituted, or a combination thereof.

The variant AAV capsid protein can comprise a deletion or substitution of the amino acid residues functionally equivalent to amino acids 452 to 577 of the AAV9 VP1 protein (SEQ ID NO: 109). The variant AAV capsid protein can comprise a deletion or substitution of the amino acid residues functionally equivalent to amino acids 433 to 640 of the AAV9 VP1 protein (SEQ ID NO: 109), amino acids 445 to 577 of the AAV9 VP1 protein (SEQ ID NO: 109), amino acids 445 to 587 of the AAV9 VP1 protein (SEQ ID NO: 109), amino acids 445 to 593 of the AAV9 VP1 protein (SEQ ID NO: 109), amino acids 445 to 594 of the AAV9 VP1 protein (SEQ ID NO: 109), amino acids 445 to 603 of the AAV9 VP1 protein (SEQ ID NO: 109), amino acids 445 to 604 of the AAV9 VP1 protein (SEQ ID NO: 109), amino acids 445 to 610 of the AAV9 VP1 protein (SEQ ID NO: 109), amino acids 445 to 611 of the AAV9 VP1 protein (SEQ ID NO: 109), amino acids 445 to 640 of the AAV9 VP1 protein (SEQ ID NO: 109), amino acids 445 to 691 of the AAV9 VP1 protein (SEQ ID NO: 109), amino acids 452 to 577 of the AAV9 VP1 protein (SEQ ID NO: 109), amino acids 452 to 581 of the AAV9 VP1 protein (SEQ ID NO: 109), amino acids 452 to 593 of the AAV9 VP1 protein (SEQ ID NO: 109), amino acids 452 to 599 of the AAV9 VP1 protein (SEQ ID NO: 109), amino acids 429 to 607 of the AAV9 VP1 protein (SEQ ID NO: 109), amino acids 428 to 640 of the AAV9 VP1 protein (SEQ ID NO: 109), or amino acids 418 to 640 of the AAV9 VP1 protein (SEQ ID NO: 109). The variant AAV capsid protein can comprise a deletion or substitution of the amino acid residues functionally equivalent to amino acids 593 to 640 of the AAV9 VP1 protein (SEQ ID NO: 109), amino acids 594 to 640 of the AAV9 VP1 protein (SEQ ID NO: 109), or amino acids 596 to 640 of the AAV9 VP1 protein (SEQ ID NO: 109). The variant AAV capsid protein can comprise a deletion or substitution of the amino acid residues functionally equivalent to amino acids 704 to 711 of the AAV9 VP1 protein (SEQ ID NO: 109), amino acids 704 to 727 of the AAV9 VP1 protein (SEQ ID NO: 109), amino acids 704 to 736 of the AAV9 VP1 protein (SEQ ID NO: 109), amino acids 706 to 736 of the AAV9 VP1 protein (SEQ ID NO: 109), amino acids 712 to 736 of the AAV9 VP1 protein (SEQ ID NO: 109), amino acids 693 to 736 of the AAV9 VP1 protein (SEQ ID NO: 109), or amino acids 692 to 736 of the AAV9 VP1 protein (SEQ ID NO: 109). The variant AAV capsid protein can comprise a deletion or substitution of the amino acid residues functionally equivalent to amino acids 658 to 667 of the AAV9 VP1 protein (SEQ ID NO: 109), or amino acids 659 to 666 of the AAV9 VP1 protein (SEQ ID NO: 109). The variant AAV capsid protein can comprise a deletion or substitution of the amino acid residues functionally equivalent to amino acids 426 to 736 of the AAV9 VP1 protein (SEQ ID NO: 109), amino acids 444 to 736 of the AAV9 VP1 protein, amino acids 445 to 736 of the AAV9 VP1 protein (SEQ ID NO: 109), amino acids 452 to 736 of the AAV9 VP1 protein (SEQ ID NO: 109), or amino acids 450 to 736 of the AAV9 VP1 protein (SEQ ID NO: 109).

In some embodiments, the substitution is by a peptide segment. In some embodiments, the peptide segment comprises no more than 20 amino acids. In some embodiments, the peptide segment is about 1, 2, 3, 4, 5, 6, 7, 8, 9, 11, 12 or 15 amino acids in length. In some embodiments, the peptide segment is a flexible peptide segment. In some embodiments, the flexible peptide segment is a G/S-rich peptide segment. In some embodiments, the flexible peptide segment is a peptide (G/S)n. In some embodiments, n is a position integer that is smaller than 21. In some embodiments, the peptide segment is, or comprises, G, GS, GG, GGS, GSG, SGGG (SEQ ID NO: 115), GGGS (SEQ ID NO: 116), GSGGG (SEQ ID NO: 117), GGGSGG (SEQ ID NO: 118), GGSGGG (SEQ ID NO: 119), SGGSGG (SEQ ID NO: 120), GGSGGS (SEQ ID NO: 121), GGSGGGS (SEQ ID NO: 122), GGGSGGG (SEQ ID NO: 123), GGSGGSG (SEQ ID NO: 124), GGGSGGGG (SEQ ID NO: 125), GGGGSGGGS (SEQ ID NO: 126), GGGGSGGGG (SEQ ID NO: 127), GGSGGSGGS (SEQ ID NO: 128), GGGSGGGSGGS (SEQ ID NO: 129), GGSGGSGGSGGS (SEQ ID NO: 130), or GGGGSGGGGSGGGGS (SEQ ID NO: 131).

In some embodiments, the variant AAV capsid protein comprises the amino acid residues functionally equivalent to Y426, A427, and H428 of the AAV9 VP1 protein (SEQ ID NO: 109). In some embodiments, the variant AAV capsid protein comprises one or more deletions, substitutions and/or insertions C-terminal to the amino acid residue functionally equivalent to H428 of the AAV9 VP1 protein (SEQ ID NO: 109).

In some embodiments, the variant AAV capsid protein comprises a deletion or substitution of the amino acid residues functionally equivalent to amino acids 659 to 666 of the AAV9 VP1 protein (SEQ ID NO: 109) and a deletion or substitution of the amino acid residues functionally equivalent to amino acids 704 to 711 of the AAV9 VP1 protein (SEQ ID NO: 109). In some embodiments, the variant AAV capsid protein comprises a substitution of the amino acid residues functionally equivalent to amino acids 659 to 666 of the AAV9 VP1 protein (SEQ ID NO: 109) by a peptide segment, and a deletion of the amino acid residues functionally equivalent to amino acids 704 to 711 of the AAV9 VP1 protein (SEQ ID NO: 109).

In some embodiments, the peptide segment is an extended hinge. In some embodiments, the peptide hinge is, or comprises, GGSGGSLCNTRN (SEQ ID NO: 132). In some embodiments, the peptide hinge is C-terminal to the amino acid residue functionally equivalent to S429 of the AAV9 VP1 protein (SEQ ID NO: 109).

In some embodiments, (a) fifty or more of the amino acid residues functionally equivalent to amino acids 452 to 581 of the AAV9 VP1 protein (SEQ ID NO: 109) have been deleted, and (b) the variant AAV capsid protein comprises five or more of the deleted amino acids in (a) in the C-terminus. In some embodiments, (a) the amino acid residues functionally equivalent to amino acids 417 to 640 of the AAV9 VP1 protein (SEQ ID NO: 109) have been substituted by a peptide segment of GGS and (b) the variant AAV capsid protein comprises the deleted amino acids 430 to 640 of the AAV9 VP1 protein (SEQ ID NO: 109) in (a) in the C-terminus. In some embodiments, (a) the amino acid residues functionally equivalent to amino acids 417 to 640 of the AAV9 VP1 protein (SEQ ID NO: 109) have been substituted by a peptide segment of GGSGGGS (SEQ ID NO: 122) and (b) the variant AAV capsid protein comprises the deleted amino acids 430 to 640 of the AAV9 VP1 protein (SEQ ID NO: 109) in (a) in the C-terminus.

Disclosed herein include variant adeno-associated virus (AAV) capsids. In some embodiments, the variant AAV capsid comprises a variant AAV capsid protein provided herein. In some embodiments, the variant AAV capsid comprises a plurality of multimers each comprising two or more AAV capsid proteins. In some embodiments, at least one of the two or more AAV capsid proteins is a variant AAV capsid protein provided herein. In some embodiments, two of the two or more AAV capsid proteins are connected by a linker. In some embodiments, the two or more AAV capsid proteins comprise VP1, VP2, VP3, derivatives thereof, or any combination thereof. In some embodiments, the variant AAV capsid comprises two or more multimers that differ with respect to the capsid protein isoforms that compose the multimers. In some embodiments, the two or more AAV capsid proteins comprise one or more parental AAV capsid proteins, or derivatives thereof. In some embodiments, the linker is a peptide linker. In some embodiments, the peptide linker comprises an amino acid sequence of GGENLYFQS (SEQ ID NO: 133). In some embodiments, the peptide linker comprises an amino acid sequence of ENLYFQG (SEQ ID NO: 134) or GGENLYFQG (SEQ ID NO: 135). In some embodiments, at least one of the two or more AAV capsid proteins is a wildtype AAV capsid protein. In some embodiments, the plurality of multimers are capable of assembling into the variant AAV capsid.

The variant AAV capsid can have an AAV serotype of AAV9, AAV2, AAV6, AAV8, or variants thereof, a hybrid or chimera of any of the foregoing AAV serotypes, and any combination thereof.

In some embodiments, the variant AAV capsid has a diameter of at least about 25 nm. In some embodiments, the variant AAV capsid has a diameter of at least about 30 nm. In some embodiments, the variant AAV capsid has a diameter of about 30 nm to about 40 nm, of about 30 nm to about 50 nm, or of about 40 nm to about 50 nm. In some embodiments, diameter is calculated as the mean of the major axis length and the minor axis length. The diameter can be measured by, for example, transmission electron microscopy (TEM) or hydrodynamic diameter. In some embodiments, the hydrodynamic diameter is measured by dynamic light scattering (DLS).

In some embodiments, the AAV capsid is capable of packing a nucleic acid more than 5.2 kb, more than 5.5 kb, more than 6 kb, more than 6.1 kb, more than 6.3 kb, or more than 6.5 kb. In some embodiments, the AAV capsid is capable of packing a nucleic acid that is about 6.7 kb in length.

The variant AAV capsid can be capable of protecting the nucleic acid from DNAse I digestion. In some embodiments, the variant AAV capsid has at least 1%, at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200%, at least 300%, at least 400%, at least 500%, at least 600%, at least 700%, at least 800%, at least 900%, or at least 1000% of free DNase I protected titer of the corresponding parental AAV capsid. The variant AAV capsid is at least about 10%, 20%, 30%, 40%, or 50% larger in diameter and/or genetic cargo capacity as compared to the corresponding parental AAV capsid and/or a wildtype AAV capsid. In some embodiments, the packaging efficiency of the variant AAV capsid is at least about 1%, 5%, 10%, 20%, 30%, 40%, or 50% of the packaging efficiency of the corresponding parental AAV capsid and/or a wildtype AAV capsid. In some embodiments, the transduction efficiency of the variant AAV capsid is at least about 0.5%, 1%, 5%, 10%, 20t 30%, 40%, 50%, 60%, 70%, 80%, 90%, 100%, 110%, 120%, 130%, 140%, or 150% of the transduction efficiency of the corresponding parental AAV capsid and/or a wildtype AAV capsid. In some embodiments, the variant AAV capsid protein comprises an amino acid sequence that is at least 80%, at least 85%, at least 90%, at least 95%, at least 98%, at least 99%, or 100% identical to a sequence of any one of SEQ ID NOs: 1-108.

Disclosed herein include recombinant AAVs (rAAVs). In some embodiments, the rAAV comprises: (a) a variant AAV capsid provided herein; and (b) a heterologous nucleic acid. In some embodiments, the heterologous nucleic acid comprises a polynucleotide encoding a payload. The payload can comprise a payload RNA agent and/or a payload protein. In some embodiments, the heterologous nucleic acid is more than 5.2 kb, more than 5.5 kb, more than 6 kb, more than 6.1 kb, more than 6.3 kb, or more than 6.5 kb in length. In some embodiments, the heterologous nucleic acid is about 6.7 kb in length. In some embodiments, the length of the heterologous nucleic acid is about 70%, about 80%, about 90%, about 95%, or about 100%, of the genetic cargo capacity of the variant AAV capsid.

In some embodiments, the heterologous nucleic acid comprises a 5′ inverted terminal repeat (ITR) and a 3′ ITR. In some embodiments, the payload comprises an RNA. In some embodiments, the payload comprises a protein. In some embodiments, the heterologous nucleic acid comprises a promoter operably linked to the polynucleotide encoding a payload. In some embodiments, the promoter is capable of inducing the transcription of the polynucleotide. In some embodiments, the heterologous nucleic acid comprises one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). In some embodiments, the silencer effector comprises a microRNA (miRNA), a precursor microRNA (pre-miRNA), a small interfering RNA (siRNA), a short-hairpin RNA (shRNA), precursors thereof, derivatives thereof, or a combination thereof. In some embodiments, said silencer effector is capable of binding the one or more silencer effector binding sequences, thereby reducing the stability of the payload transcript and/or reducing the translation of the payload transcript.

In some embodiments, the polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

The promoter can be or comprise a ubiquitous promoter. In some embodiments, the ubiquitous promoter is a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or a combination thereof.

The promoter can be an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, estrogen responsive promoter, a PPAR-γ promoter, or an RU-486 responsive promoter.

The promoter can be or comprise a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. In some embodiments, the tissue specific promoter is a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. In some embodiments, the tissue specific promoter is a muscle-specific promoter, for example a creatine kinase (MCK) promoter.

The promoter can comprise an intronic sequence. In some embodiments, the promoter comprises a bidirectional promoter and/or an enhancer (e.g., a CMV enhancer). In some embodiments, one or more cells of a subject comprise an endogenous version of the payload. In some embodiments, the promoter comprises or is derived from the promoter of the endogenous version. In some embodiments, one or more cells of a subject comprise an endogenous version of the payload. In some embodiments, the payload is not truncated relative to the endogenous version.

The payload RNA agent can comprise one or more of dsRNA, siRNA, shRNA, pre-miRNA, pri-miRNA, miRNA, stRNA, lncRNA, piRNA, and snoRNA. In some embodiments, the payload RNA agent inhibits or suppresses the expression of a gene of interest in a cell. In some embodiments, the gene of interest is selected SOD1, MAPT, APOE, HTT, C90RF72, TDP-43, APP, BACE, SNCA, ATXN1, ATXN2, ATXN3, ATXN7, SCN1A-SCN5A, or SCN8A-SCN11A.

The payload protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1 or a combination thereof.

The payload protein can comprise a disease-associated protein. In some embodiments, the level of expression of the disease-associated protein correlates with the occurrence and/or progression of the disease. The payload protein can comprise a programmable nuclease, for example Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease.

The heterologous nucleic acid can further comprises a polynucleotide encoding (i) a targeting molecule and/or (ii) a donor nucleic acid. In some embodiments, the targeting molecule is capable of associating with the programmable nuclease. In some embodiments, the targeting molecule comprises single strand DNA or single strand RNA. In some embodiments, the targeting molecule comprises a single guide RNA (sgRNA). In some embodiments, the heterologous nucleic acid further comprises a polynucleotide encoding one or more secondary proteins. In some embodiments, the payload protein and the one or more secondary proteins comprise a synthetic protein circuit. In some embodiments, the heterologous nucleic acid comprises a single-stranded AAV (ssAAV) vector or a self-complementary AAV (scAAV) vector.

In some embodiments, the rAAV has an infectivity to a host cell of at least 1%, at least 5%, at least 10%, at least 20%, at least 30%, at least 40%, at least 50%, at least 60%, at least 70%, at least 80%, or at least 90% of a wildtype AAV. In some embodiments, the rAAV has an infectivity to a host cell of at least 1%, at least 5%, at least 10%, at least 20%, at least 30%, at least 40%, at least 50%, at least 60%, at least 70%, at least 80%, or at least 90% of the corresponding wildtype AAV serotype.

Disclosed herein include compositions. In some embodiments, the composition comprises a variant AAV capsid protein provided herein, an AAV capsid provided herein, and/or an rAAV provided herein; and a pharmaceutically acceptable carrier. In some embodiments, the pharmaceutical composition is for intraventricular, intraperitoneal, intraocular, intravenous, intraarterial, intranasal, intrathecal, intracistemae magna, or subcutaneous injection, and/or for direct injection to any tissue in the body. The pharmaceutical composition provided herein can further comprise a therapeutic agent. The pharmaceutical composition provided herein can further comprise: (i) a targeting molecule or a nucleic acid encoding the targeting molecule and/or (ii) a donor nucleic acid or a nucleic acid encoding the donor nucleic acid. In some embodiments, the targeting molecule is capable of associating with the programmable nuclease. In some embodiments, the targeting molecule comprises single strand DNA or single strand RNA. In some embodiments, the targeting molecule comprises a single guide RNA (sgRNA).

Disclosed herein include methods of introducing a nucleic acid into a cell. In some embodiments, the method comprises: contacting the cell with a variant AAV capsid provided herein, or the therapeutically effective amount of the rAAV provided herein, or the composition provided herein. In some embodiments, the cell is present in a subject. In some embodiments, introducing comprises: (i) isolating one or more cells from the subject; (ii) contacting said one or more cells with a composition comprising; and (iii) administering the one or more cells into a subject after the contacting step. In some embodiments, the contacting is performed in vivo, in vitro, and/or ex vivo. In some embodiments, the contacting comprises calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the subject is a mammal. In some embodiments, the mammal is a human.

Disclosed herein include methods of treating a disease or disorder in a subject. In some embodiments, the method comprises: administering to the subject a therapeutically effective amount of an rAAV provided herein. In some embodiments, the administering comprises systemic administration. The systemic administration can be intravenous, intramuscular, intraperitoneal, or intraarticular. The administering can comprise intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or a combination thereof. In some embodiments, administering comprises an injection into a brain region. In some embodiments, administering comprises direct administration to the brain parenchyma.

The brain region can comprise the Lateral parabrachial nucleus, brainstem, Medulla oblongata, Medullary pyramids, Olivary body, or a combination thereof. In some embodiments, administering comprises delivery to dorsal root ganglia, visceral organs, astrocytes, neurons, or a combination thereof of the subject.

In some embodiments, the variant AAV capsid comprises tropism for a target tissue or a target cell. In some embodiments, the target tissue or the target cell comprises a tissue or a cell of a central nervous system (CNS) or a peripheral nervous system (PNS), or a combination thereof. The target cell can be a neuronal cell, a neural stem cell, an astrocyte, a tumor cell, a hematopoietic stem cell, an insulin producing beta cell, a lung epithelium, a skeletal cell, or a cardiac muscle cell. In some embodiments, the target cell is located in a brain or spinal cord. The target cell can be, for example, an antigen-presenting cell, a dendritic cell, a macrophage, a neural cell, a brain cell, an astrocyte, a microglial cell, and a neuron, a spleen cell, a lymphoid cell, a lung cell, or a lung epithelial cell. In some embodiments, the stem cell comprises an embryonic stem cell, an induced pluripotent stem cell (iPSC), a hematopoietic stem/progenitor cell (HSPC), or any combination thereof. The method can comprise administering an inducer of the inducible promoter to the one or more cells. In some embodiments, the inducer comprises doxycycline.

The disease or disorder can be pulmonary fibrosis, surfactant protein disorders, peroxisome biogenesis disorders, or chronic obstructive pulmonary disease (COPD). In some embodiments, the disease or disorder comprises a CNS disorder or a PNS disorder. In some embodiments, the subject is a subject suffering from or at a risk to develop one or more of chronic pain, cardiac failure, cardiac arrhythmias, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, and lysosomal storage disorders that involve cells within the CNS. In some embodiments, the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. The disease or disorder can be a neurological disease or disorder.

### BRIEF DESCRIPTION OF THE DRAWINGS

FIG. 1A-FIG. 1C depict non-limiting exemplary embodiments and data related to structural dissection of an AAV capsid subunit. FIG. 1A depicts dissection of a monomeric unit. The top bar shows the distribution of the blocks in the primary sequence of VP1. On the bottom left side are cartoon and space-filling representations of a monomer subunit in the AAV9 crystal structure (PDB ID: 3UX1); On the bottom right side are cartoon and space-filling representations of a monomer subunit modeled with Alphafold2. FIG. 1B shows different roles of the blocks in different symmetrical interactions, demonstrated with the structure of an AAV9 capsid subunit (PDB ID: 3UX1). In the bottom row pictures, the sealer block is hidden to show the interactions formed by the core block and the spike block. FIG. 1C depicts dissection of the spike block. Different segments and blocks are distinguished by the darkness.

FIG. 2A-FIG. 2B depict non-limiting exemplary embodiments and data related to comparison of AAV9 structure to TBSV. FIG. 2A depicts alignment between the core block of AAV9 (PDB ID: 3UX1) and conformation A of TBSV coat protein (PDB ID: 2TBV). FIG. 2B depicts a hypothetical T=3 architecture formed by wtAAV9 capsid proteins created using a TBSV architecture as a template. 180 copies of AAV subunits were created, and the jelly-roll folds of AAV subunits were aligned to those in a TBSV capsid structure. Finally, the diameter of the whole capsid was expanded by 10% because of the ˜10% size difference between the jelly rolls of AAV subunits and TBSV subunits. Note that the residues at the 3-fold interfaces (mainly spike block residues) have spatial conflicts with each other. The conformations A, B and C in a TBSV capsid are distinguished by the darkness of the color and indicated by arrows.

FIG. 3 depicts non-limiting exemplary embodiments and data related to AlphaFold2-predicted structure of 6 subunits of AAV9 Δ426-736. No PDB template was supplied as an input. The capsid proteins were predicted to fold in a similar way as wild-type AAV/TBSV capsids and form a planar hexamer.

FIG. 4A-FIG. 4B depict non-limiting exemplary embodiments and data related to genome-protecting structures formed by C-terminal-truncated capsid variants. FIG. 4A depicts DNaseI-protected qPCR titer provided by a series of C-terminus-truncated capsid proteins. Variants that are truncated at ˜450th amino acid, close to the boundary of one spike “arm” and the spike “tip” showed the highest titer (3-4 fold compared to wtAAV9) when packaging a 6.7 kb genome. Another peak is at the truncated site the ˜593th amino acid, the boundary of the other spike “arm” and the other spike “tip”. FIG. 4B shows morphology of aggregated AAV9 Δ450-736 capsids under negative stain TEM. Note the scale bar lengths differ in the three images.

FIG. 5 depicts non-limiting exemplary embodiments of PyMOL-modeled structure of the trimer formed by wtAAV9 and AAV9 Δ450-736. The left column shows the structure of a native AAV trimer (PDB ID: 3UX1); the middle column shows a trimeric structure where the orientation of each monomer is aligned to that of a TBSV monomer; the right column shows the TBSV-templated trimer structure of the truncated AAV9 capsid protein containing a deletion of AAs 450-736. As the comparison shows, the deletion of the bulky spike block allows forming “flatter” trimers, which is needed for spherical capsids with larger radius of curvature. In the meantime, the “arm” residues (arrows, residues 429-444) help forming simple 3-fold interactions needed for capsid formation.

FIG. 6A-FIG. 6B depict non-limiting exemplary embodiments and data related to structure and capsid morphology of a core-block-only capsid variant. FIG. 6A depicts AF2-predicted structure of AAV9 Δ410-653 GGS Δ691-736 monomer, aligned with an AAV9 monomer structure (PDB ID: 3ux1). FIG. 6B depicts negative stain TEM micrograph of the purified capsids. Scale bar in the left image: 200 nm; scale bar in the right image: 100 nm.

FIG. 7A-FIG. 7B depict non-limiting exemplary embodiments and data related to design and morphology of a tandem-dimer capsid with truncated subunits. FIG. 7A shows a diagram showing the design, where the lighter color represents deleted residues mapped to a AAV9 structure model. FIG. 7B shows negative stain TEM micrograph of the purified capsids. Scale bar in the left image: 200 nm; scale bar in the right image: 100 nm.

FIG. 8 depicts non-limiting exemplary embodiments and data related to the “arm” and “tip” fragments of the spike block predicted to independently fold into a globular domain with mostly hydrophilic surfaces. AF-2 predicted structures of AAV9 residues 429-607 are shown. In the top right model, the shading indicates the hydrophobicity of the surface residues, where the darker shading represents higher hydrophobicity.

FIG. 9A-FIG. 9D depict non-limiting exemplary embodiments and data related to design and TEM morphology of a few capsid-forming spike-deletion variants based on AAV9 backbone. FIG. 9A depicts design and TEM morphology of capsid formed by protein AAV9 Δ433-640 [GS7]. FIG. 9B depicts design and TEM morphology of capsid formed by protein AAV9 Δ445-610 [GS9]. FIG. 9C depicts design and TEM morphology of capsid formed by protein AAV9 Δ452-599. FIG. 9D depicts capsid formed by protein AAV9 Δ445-691 [Native14mer]. In each of FIG. 9A-FIG. 9D, the left side shows the design mapped to an AAV9 crystal structure (PDB ID: 3UX1) (lighter shading: residues that are deleted, darker shading: residues that are kept). Scale bars, 100 nm.

FIG. 10A-FIG. 10G depict non-limiting exemplary embodiments and data related to rational deletions in the sealer block that do not change the morphology or yield of the capsids. FIG. 10A shows negative stain TEM images of wild-type AAV9. FIG. 10B shows negative stain TEM images of AAV9 Δ704-711 [(GS)₃] capsid. FIG. 10C shows negative stain TEM images of AAV9 Δ659-666 [GS] capsid. FIG. 10D depicts negative stain TEM images of AAV9 Δ659-666 [GS] Δ704-711 [(GS)₃] capsid. FIG. 10E depicts cartoon indicating the locations of the deleted loops. FIG. 10F depicts DNaseI-protected genome titer (vg) of sealer-deleted variants when packaging at full-capacity (5.2 kb genome) or with an oversized (6.7 kb) genome. Note the y-axis is in log-scale. FIG. 10G depicts negative stain TEM images of capsids formed by a tandem-dimer unit of a wild-type AAV9 subunit linked to a sealer-truncated variant. Scale bars, 100 nm.

FIG. 11A-FIG. 11D depict non-limiting exemplary embodiments and data related to extension of the “428 hinge” for increased infectious titer of truncated capsids. FIG. 11A shows structural analysis highlighting the critical role of the hinge near the 428th residue in determining the curvature of the trimer formed by a capsid subunit without intertwined 3-fold interactions. FIG. 11B-FIG. 11D depict infectious titer assay results indicating the relative number of infectious particles in the media of virus producer cells transfected with different capsid DNA. Briefly, AAV capsids carrying a 6.1 kb CAG-GFP-CMV-mCherry double-fluorophore rAAV genome (SEQ ID NO: 110) were produced with the standard triple-transfection method in HEK293T cells. 3 days post transfection, the media of the producer cells were collected and treated with DNaseI (0.1 U/μL enzyme, 1× buffer, 37° C. 1-2 hr) as well as thermal ablation (45° C., 1 hr). 50 uL of treated media were then used to infect reporter cells pre-transfected with pHelper plasmids. Images were taken 4 days after infection. FIG. 11B shows that adding a 6mer flexible peptide insertion helps improving the infectious titer of C-terminal-truncated variants, particularly the capsid with Δ450-736 variant in an AAV-DJ backbone. FIG. 11C shows that flexible peptides with different lengths were inserted after the 428^{th} residue in the C-terminal-truncated variants of AAV-DJ. FIG. 11D shows that flexible peptides with different lengths were inserted after the 428^{th} residue in the C-terminal-truncated variants of AAV-DJ. Scale bar, 500 μm.

FIG. 12A-FIG. 12B depict non-limiting exemplary embodiments and data showing rationally designed capsid variants yield comparable infectious titer to wild-type AAV capsids when packaging an oversized (6.1 kb) genome. The infectious titer assay is the same as described above for FIG. 11A-FIG. 11D. FIG. 12A depicts variants that combined spike deletion and “hinge” extension. FIG. 12B depicts variants that combined spike deletion and one or two sealer deletions. Note that all variants in FIG. 12B transduced some cells with two fluorophores at the same time, indicating expression from a full-length oversized genome. Scale bar, 500 μm.

FIG. 13A-FIG. 13B depict non-limiting exemplary embodiments and data showing mechanism of curvature reduction by the truncations in the spike block and the sealer block. FIG. 13A depicts that truncations in the spike block can help remove the steric hindrance against reducing curvature within a trimer. FIG. 13B depicts that truncations in the sealer block can help reduce the curvature between the trimers. These mechanisms also suggest other ways of reducing the curvature instead of truncation, for example by adding flexible linkers between the core block and the “hindering” block (i.e. the spike block or the sealer block).

FIG. 14 depicts non-limiting exemplary embodiments and data related to example architectures of icosahedral capsids. Each icosahedral capsid is made of 60×T subunits. Representative symmetrical interactions between the subunits involved in each type of capsid are shown in the small spheres. Among the symmetrical interactions, the 5-fold interaction (black curves in the sectional view) is the major source of curvature in an icosahedral capsid. (Pseudo-) 6-fold interaction (orange lines in the sectional view), which only appears in T>3 capsids, results in planar hexamers that connect the 5-fold vertices. These extra subunits between the 5-fold vertices contribute to the expansion of the capsid's size. Structures used for visualizations throughout this figure are low-resolution models of a T=1 deletion mutant of Sesbania mosaic viral capsid (PDB ID: 1VAK) and the T=3 wild-type capsid of the same species (PDB ID: 1SMV). Dark shading, light shading, and white represent three minorly different conformations taken by the chemically identical subunits in a T=3 capsid.

FIG. 15A-FIG. 15C depict non-limiting exemplary embodiments and data related to quality control of AF2-modeled structures. FIG. 15A depicts AF2 predicted structures ranked by average IDDT. FIG. 15B depicts number of MSA sequences (top) and IDDT score (bottom) at each residue position. FIG. 15C depicts PAE plot of the 5 predicted models.

FIG. 16A-FIG. 16D depict non-limiting exemplary embodiments and data related to structure analysis and modeling indicating that 3-fold and 2-fold interactions restrict the curvature of AAV capsids. FIG. 16A depicts AAV capsid structure (PDB ID: 3UX1) colored by groups of pentamers. The polygon symbols indicate the 2-, 3-, 5-fold symmetric axes. Residues that are involved in 3-fold interactions and 2-fold interactions, cementing the inter-pentamer angles, were indicated in dark shading and also marked with “*”. FIG. 16B-FIG. 16D depict a hallucinated T=3 AAV capsid architecture modeled by aligning individual AAV capsid proteins into a T=3 TBSV structure (PDB ID: 2TBV) followed by even expansion by 1.1-fold. FIG. 16B-FIG. 16C depict comparison of the model and wt AAV9 structure (PDB ID: 3UX1) showing that 3-fold/2-fold interactions can create intra-trimeric/inter-trimetric steric hindrance against AAV capsids from bending into a lower curvature. The dark dots indicate steric clashes in the models. FIG. 16D depicts overall view of the model confirms with circles highlighting the major sites of steric clashes.

FIG. 17A-FIG. 17C depict non-limiting exemplary embodiments and data related to the intertwined 3-fold interactions around the spikes that can cement the curvature of AAV capsids and can tolerate large deletions. FIG. 17A depicts schematic showing that the 3-fold interactions around the spikes can prevent AAV capsids from adopting a lower curvature. FIG. 17B depicts structural alignment between AAV9 (PDB ID: 3UX1, grey), an invertebrate parvovirus GmDNV (PDB ID: 1DNV, light color), and an AlphaFold-modeled AAV9 variant with trimmed 3-fold spike region. FIG. 17C depicts alignment of sequences of VP1 capsid proteins from ˜100 AAV serotypes. The level of conservativeness at each position is indicated by darkness of a scale bar on top of the sequences (the dark color the highlighted sequences: most conserved; the dark color without highlights of the sequences: least conserved). The consensus sequence, AAV9 VP1 sequence, and AAV-DJ VP1 sequence are shown for reference.

FIG. 18A-FIG. 18C depict non-limiting exemplary embodiments and data related to characterization of AAV9 Δ445-610 capsid. FIG. 18A depicts Western blot using the media of producer cells of a few AAV9 variants. Primary antibody: mouse anti-VP1 antibody (clone A1). AAV9 Δ445-611 capsid showed a band at a smaller size as expected. FIG. 18B depicts Negative-stain TEM image of capsids purified with a precipitation-based method. Scale bars, 200 nm. FIG. 18C depicts DNaseI-protected qPCR titering of the capsid variants.

FIG. 19A-FIG. 19B depict non-limiting exemplary embodiments and data related to characterization of AAV-DJ Δ445-594 capsid and derivatives. FIG. 19A depicts Western blot using the cell lysate of producer cells of a few AAV9 variants. Primary antibody: mouse anti-VP1 antibody (clone A1). The triple band pattern that appeared in every variant can be a result of protein degradation. FIG. 19B depicts negative-stain TEM image of capsids purified with a precipitation-based method. Scale bars, 200 nm.

FIG. 20A-FIG. 20B depict non-limiting exemplary embodiments and data related to some spike-trimmed capsids that provide genome protection against free DNaseI and can transduce cultured HEK293T cells. FIG. 20A depicts relative genome protection assay results. Lysates of HEK293T cells producing different capsid variants were aliquoted, and each aliquot was treated with 80 U/mL free recombinant DNase I or immobilized recombinant DNase I (in the form of agarose resin suspensions) at 37° C. on a shaker overnight. The samples were then titered in triplicates. Relative genome protection against free DNase I was calculated by dividing free-DNase-I-treated qPCR titer by immobilized-DNase-I-treated qPCR titer. Black arrow: positive control. White arrow: negative control. FIG. 20B depicts infectious titer assay results. Briefly, AAV capsids carrying a 6.3 kb EF1a-mCherry-IRES-Cre-IRES-EGFP double-fluorophore rAAV genome (SEQ ID NO: 111) were produced with the standard triple-transfection method in HEK293T cells. 3 days post-transfection, the media of the producer cells were collected and treated with DNaseI (0.1 U/μL enzyme, 1× buffer, 37° C. 1-2 hr) as well as thermal ablation (45° C., 1 hr). 50 μL of treated media were then used to infect reporter cells pre-transfected with pHelper plasmids. Images were taken 3 days after infection. Scale bar, 500 μm.

FIG. 21 depicts a non-limiting exemplary workflow for iterative screening in 96-well format.

FIG. 22 depicts non-limiting exemplary embodiments and data related to AlphaFold2 models of a few AAV9 variants with or without an extension at the hinge around residue 428.

FIG. 23 depicts non-limiting exemplary embodiments related to the structure of pAAV-CAG-GFP-spacer-CMV-mCherry (SEQ ID NO: 110).

FIG. 24 depicts non-limiting exemplary embodiments related to the structure of pAAV-EF1a-mCh-IRES-Cre-IRES-EGFP (SEQ ID NO: 111).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include variant adeno-associated virus (AAV) capsid proteins. In some embodiments, the variant AAV capsid protein comprises: (a) fifty or more of amino acid residues functionally equivalent to amino acids 452 to 577 of the AAV9 VP1 protein (SEQ ID NO: 109) have been deleted or substituted, (b) twenty or more of amino acid residues functionally equivalent to amino acids 597 to 640 of the AAV9 VP1 protein (SEQ ID NO: 109) have been deleted or substituted, (c) five or more of amino acid residues functionally equivalent to amino acids 656 to 669 of the AAV9 VP1 protein (SEQ ID NO: 109) have been deleted or substituted, (d) five or more of amino acid residues functionally equivalent to amino acids 692 to 736 of the AAV9 VP1 protein (SEQ ID NO: 109) have been deleted or substituted, or a combination thereof.

Disclosed herein include variant adeno-associated virus (AAV) capsids. In some embodiments, the variant AAV capsid comprises a variant AAV capsid protein provided herein. In some embodiments, the variant AAV capsid comprises a plurality of multimers each comprising two or more AAV capsid proteins. Disclosed herein include recombinant AAVs (rAAVs). In some embodiments, the rAAV comprises: (a) a variant AAV capsid provided herein; and (b) a heterologous nucleic acid. In some embodiments, the heterologous nucleic acid comprises a polynucleotide encoding a payload. In some embodiments, the payload comprises a payload RNA agent and/or a payload protein. Disclosed herein include compositions. In some embodiments, the composition comprises a variant AAV capsid protein provided herein, an AAV capsid provided herein, and/or an rAAV provided herein; and a pharmaceutically acceptable carrier.

Disclosed herein include methods of introducing a nucleic acid into a cell. The method can comprise: contacting the cell with a variant AAV capsid provided herein, or the therapeutically effective amount of the rAAV provided herein, or the composition provided herein. Disclosed herein include methods of treating a disease or disorder in a subject, comprising: administering to the subject a therapeutically effective amount of an rAAV provided herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

As used herein, the term “plasmid” refers to a nucleic acid that can be used to replicate recombinant DNA sequences within a host organism. The sequence can be a double stranded DNA.

As used herein, the term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene, for example, AAV9 Δ659-666GS, Δ704-727 and AAV-DJ Δ445-594. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise, an AAV can infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental AAV capsid that has been modified in an amino acid sequence of the parental AAV capsid protein.

The term “AAV genome” as used herein refers to nucleic acid polynucleotide encoding genetic information related to the virus. The genome, in some instances, comprises a nucleic acid sequence flanked by AAV inverted terminal repeat (ITR) sequences. The AAV genome can be a recombinant AAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV ITR sequences. In some embodiments, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein can be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” refers to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™ Auxiliary, stabilizer, emulsifier, lubricant, binder, pH adjustor controller, isotonic agent and other conventional additives can also be added to the carriers.

Provided herein are methods for creating AAV capsids. In some embodiments, the method comprises complete or partial deletion/substitution from the C-terminus of a wild-type or engineered capsid protein, for example to create a capsid variant with deletion of residues 444-736, residues 450-736, residues 452-736, or residues 593-736 in AAV9.

Disclosed herein includes a method to create AAV capsids by using the whole or a part of the “core block” (Table 4) as a backbone, optionally with the insertion of a non-AAV-derived peptide or a short stretch (<200 aa) of AAV-derived peptide. Also disclosed includes a method to create AAV capsids by complete or partial deletion/substitution of the “spike block” (Table 4) from a wild-type or engineered capsid protein. For example, to create a size-expanded capsid variant, AAV9 Δ433-640 (G/S)7, from wtAAV9 by deleting residues 433-640 and inserting GGGSGGS sequence (SEQ ID NO: 136) at the deletion site.

Some embodiments provide a method to create AAV capsids, in which the target region starts or ends with sites such as block boundaries, segment boundaries, or the sites of the hinges described in Table 4 and Table 5. In some embodiments, the present disclosure provided a method to create AAV capsids by complete or partial deletion/substitution of the “sealer block” from a wild-type or engineered capsid protein. Provided herein include a method to create AAV capsids by insertion(s) of G/S-rich flexible linkers to a parent capsid protein (e.g., a wild-type or engineered capsid protein). Provided herein includes a method to create AAV capsids, in which the insertion sites are at the boundaries between the blocks and segments (Table 4) or the “hinge” sites (Table 5). Also provided includes a method to create AAV capsids by tandemly linking the coding sequence for two subunits of wild-type or engineered capsid proteins together, optionally with a peptide linker in between. The individual subunits can be either a wild-type AAV capsid or an engineered capsid. Some embodiments provide a method to create AAV capsids using designs or sequences described herein (See, Example 1 and Example 2; Table 3-Table 4 below).

Also provided includes a method to create AAV capsids by combining one or more of the methods described herein. Provided herein includes a method to create size-expanded (diameter>28 nm) capsids, for example with any of methods above. The AAV capsids created by methods described herein can be used, for example, as delivery vectors of DNA sequences, as antigen-display platforms, or as components of other biologics, both in vitro and in vivo.

### Structure-Guided Design of Variant AAV Capsid Proteins and Capsids

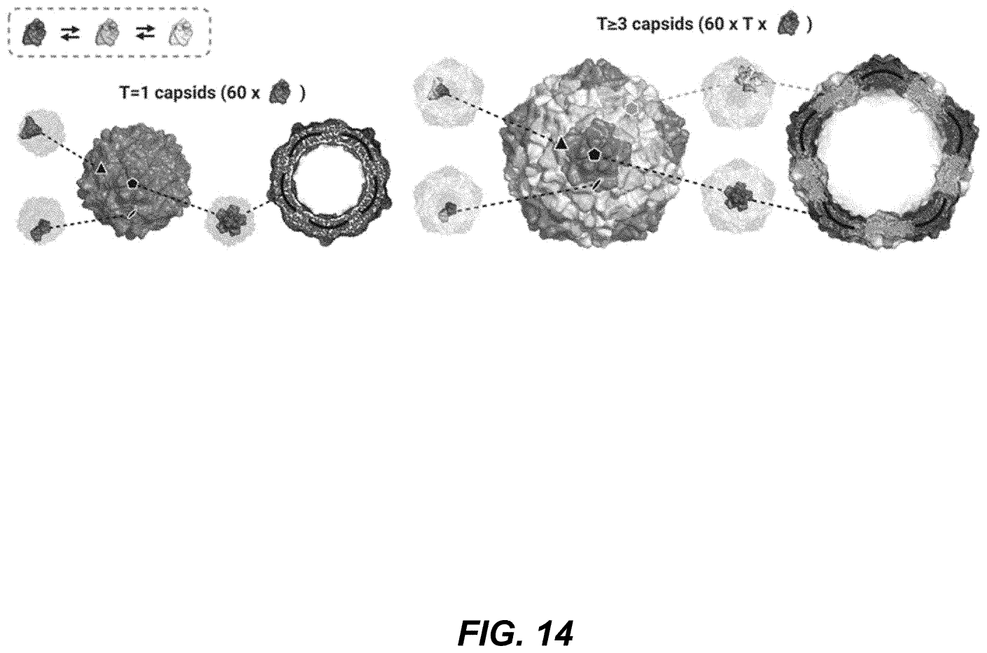

The geometry of an icosahedral viral capsid is described by its triangulation (T) number. By this definition, a viral capsid is built from 60T subunits with symmetrical interactions. For example, a T=1 capsid like wild-type AAV comprises 60 subunits, whereas a T=3 capsid would comprise 180 subunits, as shown in FIG. 14. Capsid size polymorphism has been reported in many natural icosahedral capsids. Some icosahedral capsids, notably a number of ssRNA plant viral capsids that share similar jelly-roll protein folds as AAV capsids, can form spherical particles of different sizes (T numbers) with only minor sequence changes. The larger forms of these capsids are produced by organizing a greater number of identical subunits into more complicated icosahedral geometries. Inspired by this phenomenon, it is provided herein that the size of AAV capsids was expanded by adopting these more complicated geometries.

Guided by a structural dissection, rational modifications of the capsids, such as deletions and substitutions of capsid sequences or insertion of short linkers, can lead to genome-protecting capsids with distinct sizes and morphologies. The AAV capsid subunit has been structurally dissected into 4 blocks: disordered N-terminus block (residues 1-218), core block (residues 219-417, 641-655, 670-691), spike block (residues 418-640), and sealer block (residues 656-669, 692-736). Except for the disordered N-terminus block, each block plays a unique role in capsid assembly. The minimal sequence required for forming genome-protecting assemblies resides in the core block, while the spike block and the sealer block modulate the morphology and size of the assembly products. Structure-guided deletions and substitutions in the spike block and the sealer block result in capsid-forming variants with diameters larger than 30 nm. This size switching caused by deletions and substitutions in the spike block or the sealer block can be through reducing the surface curvature within a trimer or between neighboring trimers, respectively. In addition, key “engineerable” sites were identified for modulating AAV capsid assembly, such as the boundaries between the blocks and segments as well as internal “hinges”. Insertions at these key sites or deletions between the sites can lead to capsids with expanded sizes. The structural dissection can serve as a useful guide for future engineering of AAV capsid assembly. Without being bound by any particular theory, the overall curvature of wild-type AAV trimers, the basic building block of AAV capsids, is restricted by the bulky, interlacing 3-fold interaction. The bulky, interlacing 3-fold interaction can sterically prevent AAV trimers from bending and adapting to a larger radius of curvature. As described herein, AAV capsid variants can be designed with structure-guided deletions and substitutions in the spike-forming 3-fold interaction region. Some variants resulted in viral particles with diameters around 40 nm. Structure-guided modifications resulted in variants with improved production yield. These viral particles can provide partial protection to encapsidated rAAV genomes and deliver to cultured cells in vitro, albeit at low efficiency. These size-expanded capsids can deliver conventionally “oversized” cargos that are larger than 5 kb. The rationally designed capsid variants and their derivatives can serve as novel delivery vectors for gene therapy.

Disclosed herein include variant AAV capsid proteins, in which (a) fifty or more of amino acid residues functionally equivalent to amino acids 452 to 577 of the AAV9 VP1 protein (SEQ ID NO: 109) have been deleted or substituted, (b) twenty or more of amino acid residues functionally equivalent to amino acids 597 to 640 of the AAV9 VP1 protein (SEQ ID NO: 109) have been deleted or substituted, (c) five or more of amino acid residues functionally equivalent to amino acids 656 to 669 of the AAV9 VP1 protein (SEQ ID NO: 109) have been deleted or substituted, (d) five or more of amino acid residues functionally equivalent to amino acids 692 to 736 of the AAV9 VP1 protein (SEQ ID NO: 109) have been deleted or substituted, or a combination thereof.

In some embodiments of the variant AAV capsid proteins disclosed herein, (a) one hundred or more of amino acid residues functionally equivalent to amino acids 452 to 577 of the AAV9 VP1 protein (SEQ ID NO: 109) have been deleted or substituted, (b) thirty or more of amino acid residues functionally equivalent to amino acids 596 to 640 of the AAV9 VP1 protein (SEQ ID NO: 109) have been deleted or substituted, (c) ten or more of amino acid residues functionally equivalent to amino acids 656 to 669 of the AAV9 VP1 protein (SEQ ID NO: 109) have been deleted or substituted, (d) twenty or more of amino acid residues functionally equivalent to amino acids 692 to 736 of the AAV9 VP1 protein (SEQ ID NO: 109) have been deleted or substituted, or a combination thereof. In some embodiments of the variant AAV capsid proteins disclosed herein, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126 amino acid residues functionally equivalent to amino acids 452 to 577 of the AAV9 VP1 protein (SEQ ID NO: 109) have been deleted or substituted. In some embodiments of the variant AAV capsid proteins disclosed herein, 20, 21, 22, 23, 24, 25, 26, 27, 28, 29, 30, 31, 32, 33, 34, 35, 36, 37, 38, 39, 40, 41, 42, 43, 44, or 45 amino acid residues functionally equivalent to amino acids 596 to 640 of the AAV9 VP1 protein (SEQ ID NO: 109) have been deleted or substituted. In some embodiments of the variant AAV capsid proteins disclosed herein, 5, 6, 7, 8, 9, 10, 11, 12, 13, or 14 amino acid residues functionally equivalent to amino acids 656 to 669 of the AAV9 VP1 protein (SEQ ID NO: 109) have been deleted or substituted. In some embodiments of the variant AAV capsid proteins disclosed herein, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acid residues functionally equivalent to amino acids 692 to 736 of the AAV9 VP1 protein (SEQ ID NO: 109) have been deleted or substituted.

The variant AAV capsid protein can comprise a deletion or substitution of the amino acid residues functionally equivalent to amino acids 452 to 577 of the AAV9 VP1 protein (SEQ ID NO: 109). The variant AAV capsid protein can comprise a deletion or substitution of the amino acid residues functionally equivalent to amino acids 433 to 640 of the AAV9 VP1 protein (SEQ ID NO: 109), amino acids 445 to 577 of the AAV9 VP1 protein (SEQ ID NO: 109), amino acids 445 to 587 of the AAV9 VP1 protein (SEQ ID NO: 109), amino acids 445 to 593 of the AAV9 VP1 protein (SEQ ID NO: 109), amino acids 445 to 594 of the AAV9 VP1 protein (SEQ ID NO: 109), amino acids 445 to 603 of the AAV9 VP1 protein (SEQ ID NO: 109), amino acids 445 to 604 of the AAV9 VP1 protein (SEQ ID NO: 109), amino acids 445 to 610 of the AAV9 VP1 protein (SEQ ID NO: 109), amino acids 445 to 611 of the AAV9 VP1 protein (SEQ ID NO: 109), amino acids 445 to 640 of the AAV9 VP1 protein (SEQ ID NO: 109), amino acids 445 to 691 of the AAV9 VP1 protein (SEQ ID NO: 109), amino acids 452 to 577 of the AAV9 VP1 protein (SEQ ID NO: 109), amino acids 452 to 581 of the AAV9 VP1 protein (SEQ ID NO: 109), amino acids 452 to 593 of the AAV9 VP1 protein (SEQ ID NO: 109), amino acids 452 to 599 of the AAV9 VP1 protein (SEQ ID NO: 109), amino acids 429 to 607 of the AAV9 VP1 protein (SEQ ID NO: 109), amino acids 428 to 640 of the AAV9 VP1 protein (SEQ ID NO: 109), or amino acids 418 to 640 of the AAV9 VP1 protein (SEQ ID NO: 109). The variant AAV capsid protein can comprise a deletion or substitution of the amino acid residues functionally equivalent to amino acids 593 to 640 of the AAV9 VP1 protein (SEQ ID NO: 109), amino acids 594 to 640 of the AAV9 VP1 protein (SEQ ID NO: 109), amino acids 596 to 640 of the AAV9 VP1 protein (SEQ ID NO: 109).

The variant AAV capsid protein can comprise a deletion or substitution of the amino acid residues functionally equivalent to amino acids 704 to 711 of the AAV9 VP1 protein (SEQ ID NO: 109), amino acids 704 to 727 of the AAV9 VP1 protein (SEQ ID NO: 109), amino acids 704 to 736 of the AAV9 VP1 protein (SEQ ID NO: 109), amino acids 706 to 736 of the AAV9 VP1 protein (SEQ ID NO: 109), amino acids 712 to 736 of the AAV9 VP1 protein (SEQ ID NO: 109), amino acids 693 to 736 of the AAV9 VP1 protein (SEQ ID NO: 109), or amino acids 692 to 736 of the AAV9 VP1 protein (SEQ ID NO: 109). The variant AAV capsid protein can comprise a deletion or substitution of the amino acid residues functionally equivalent to amino acids 658 to 667 of the AAV9 VP1 protein (SEQ ID NO: 109), or amino acids 659 to 666 of the AAV9 VP1 protein (SEQ ID NO: 109). The variant AAV capsid protein can comprise a deletion or substitution of the amino acid residues functionally equivalent to amino acids 426 to 736 of the AAV9 VP1 protein (SEQ ID NO: 109), amino acids 444 to 736 of the AAV9 VP1 protein, amino acids 445 to 736 of the AAV9 VP1 protein (SEQ ID NO: 109), amino acids 452 to 736 of the AAV9 VP1 protein (SEQ ID NO: 109), or amino acids 450 to 736 of the AAV9 VP1 protein (SEQ ID NO: 109).

The substitution can be by a peptide segment (e.g., a flexible linker). The peptide segment can comprise no more than 20 (e.g., 1, 2, 3, 4, 5, 6, 7, 8, 9, 10, 11, 12, 13, 14, 15, 16, 17, 18, 19, or 20) amino acids. The peptide segment can be a flexible peptide segment. The flexible peptide segment can be a G/S-rich peptide segment. The flexible peptide segment can be a peptide (G/S)n, in which n is a position integer that can be smaller than 21. For example, n is 1, 2, 3, 4, 5, 6, 7, 8, 9, 10, 11, 12, 13, 14, 15, 16, 17, 18, 19, or 20. The peptide segment can be, or can comprise, G, GS, GG, SG, SS, GGS, GSG, GSS, SGG, GGG, SSS, SGS, SGG, SSG, SGGG (SEQ ID NO: 115), GGGS (SEQ ID NO: 116), GSGGG (SEQ ID NO: 117), GGGSGG (SEQ ID NO: 118), GGSGGG (SEQ ID NO: 119), SGGSGG (SEQ ID NO: 120), GGSGGS (SEQ ID NO: 121), GGSGGGS (SEQ ID NO: 122), GGGSGGG (SEQ ID NO: 123), GGGSGGS (SEQ ID NO: 136), GGSGGSG (SEQ ID NO: 124), GGGSGGGG (SEQ ID NO: 125), GGGGSGGGS (SEQ ID NO: 126), GGGGSGGGG (SEQ ID NO: 127), GGSGGSGGS (SEQ ID NO: 128), GGGSGGGSGGS (SEQ ID NO: 129), GGSGGSGGSGGS (SEQ ID NO: 130), or GGGGSGGGGSGGGGS (SEQ ID NO: 131).

The variant AAV capsid protein can comprise the amino acid residues functionally equivalent to Y426, A427, and H428 of the AAV9 VP1 protein (SEQ ID NO: 109). The variant AAV capsid protein can comprise one or more deletions, substitutions and/or insertions C-terminal to the amino acid residue functionally equivalent to H428 of the AAV9 VP1 protein (SEQ ID NO: 109).

The variant AAV capsid protein can comprise a deletion or substitution of the amino acid residues functionally equivalent to amino acids 659 to 666 of the AAV9 VP1 protein (SEQ ID NO: 109) and a deletion or substitution of the amino acid residues functionally equivalent to amino acids 704 to 711 of the AAV9 VP1 protein (SEQ ID NO: 109). The variant AAV capsid protein can comprise a substitution of the amino acid residues functionally equivalent to amino acids 659 to 666 of the AAV9 VP1 protein (SEQ ID NO: 109) by a peptide segment, and a deletion of the amino acid residues functionally equivalent to amino acids 704 to 711 of the AAV9 VP1 protein (SEQ ID NO: 109).

The peptide segment can be an extended hinge. The peptide hinge can be, or comprise, GGSGGSLCNTRN (SEQ ID NO: 132). The peptide hinge can be C-terminal to the amino acid residue functionally equivalent to 5429 of the AAV9 VP1 protein (SEQ ID NO: 109).

In some embodiments of the variant AAV capsid proteins disclosed herein, (a) fifty or more of the amino acid residues functionally equivalent to amino acids 452 to 581 of the AAV9 VP1 protein (SEQ ID NO: 109) have been deleted, and (b) the variant AAV capsid protein can comprise five or more of the deleted amino acids in (a) in the C-terminus. In some embodiments of the variant AAV capsid proteins disclosed herein, (a) the amino acid residues functionally equivalent to amino acids 417 to 640 of the AAV9 VP1 protein (SEQ ID NO: 109) have been substituted by a peptide segment of GGS and (b) the variant AAV capsid protein can comprise the deleted amino acids 430 to 640 of the AAV9 VP1 protein (SEQ ID NO: 109) in (a) in the C-terminus. In some embodiments of the variant AAV capsid proteins disclosed herein, (a) the amino acid residues functionally equivalent to amino acids 417 to 640 of the AAV9 VP1 protein (SEQ ID NO: 109) have been substituted by a peptide segment of GGSGGGS (SEQ ID NO: 122) and (b) the variant AAV capsid protein can comprise the deleted amino acids 430 to 640 of the AAV9 VP1 protein (SEQ ID NO: 109) in (a) in the C-terminus.

Disclosed herein include variant adeno-associated virus (AAV) capsids. In some embodiments the variant AAV capsids comprise a variant AAV capsid protein provided herein. The AAV capsid can comprise a plurality of multimers each comprising two or more AAV capsid proteins. At least one of the two or more AAV capsid proteins can be a variant AAV capsid protein provided herein. Two of the two or more AAV capsid proteins can be connected by a linker. The two or more AAV capsid proteins can comprise VP1, VP2, VP3, derivatives thereof, or any combination thereof. The variant AAV capsid can comprise two or more multimers that differ with respect to the capsid protein isoforms that compose the multimers. The two or more AAV capsid proteins can comprise one or more parental AAV capsid proteins, or derivatives thereof. The plurality of multimers can assemble into the variant AAV capsid.

The variant AAV capsid can comprise VP1, VP2, and/or VP3. The variant AAV capsid can comprise an about 1:1:10 ratio of VP1:VP2:VP3. The structure of the variant AAV capsid can retain at least one surface epitope present on the corresponding parental AAV capsid. The at least one surface epitope can be responsible for targeting the variant AAV capsid to one or more cell types. The variant AAV capsid can be capable of being purified with an antigen-binding fragment versus the corresponding parental AAV capsid. The variant AAV capsid and/or the corresponding parental AAV capsid can comprise an icosahedral geometry. The VP1 of AAV9 can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 109. Amino acid residue positions provided herein are in reference to the sequence of VP1.

The linker can be a peptide linker. At least one peptide linker can comprise an amino acid sequence of GGENLYFQS (SEQ ID NO: 133). The multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NOs: 8 and 23. At least one peptide linker can comprise an amino acid sequence of ENLYFQG (SEQ ID NO: 134) or GGENLYFQG (SEQ ID NO: 135).

At least one of the two or more AAV capsid proteins can be a wildtype AAV capsid protein. The variant AAV capsid can have an AAV serotype of AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 A1 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-1 1/rh.53, AAV4-8/rl 1.64, AAV4-9/rh.54, AAV4-19/rh.55, AAV5-3/rh.57, AAV5-22/rh.58, AAV7.3/hu.7, AAV16.8/hu. 10, AAV16.12/hu.11, AAV29.3/bb 0.1, AAV29.5/bb 0.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33 0.4/hu. 15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi. 1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu. 13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t19, AAVrh.2, AAVrh.2R, AAVrh.8, AAVrh.8R, AAVrh.10, AAVrh.12, AAVrh. 13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The variant AAV capsid can have a diameter of at least about 25 nm. The variant AAV capsid can have a diameter of at least about 30 nm. The variant AAV capsid can have a diameter of about 30 nm to about 35 nm, of about 30 nm to about 60 nm, of about 40 nm to about 60 nm, of about 45 nm to about 60 nm, of about 50 nm to about 65 nm, or of about 55 nm to about 60 nm, of about 20 nm to about 100 nm, of about 20 nm to about 80 nm, of about 20 nm to about 60 nm, of about 20 nm to about 40 nm, of about 30 nm to about 100 nm, of about 30 nm to about 80 nm, of about 30 nm to about 60 nm, of about 20 nm to about 40 nm, of about 40 nm to about 100 nm, of about 40 nm to about 80 nm, of about 40 nm to about 60 nm, of about 50 nm to about 100 nm, of about 50 nm to about 80 nm, of about 50 nm to about 60 nm, of about 60 nm to about 100 nm, or of about 60 nm to about 80 nm, or a number or a range between any two of these values. The diameter of a variant capsid can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The capsid particles demonstrate a variation in size. The diameter can be calculated as the mean of the major axis length and the minor axis length. The diameter can be measured by transmission electron microscopy (TEM). The diameter can be hydrodynamic diameter, e.g., measured by dynamic light scattering (DLS).

The genetic cargo capacity of the variant AAV capsids provided herein can vary. The AAV capsid can pack a nucleic acid more than 5.2 kb, more than 5.5 kb, more than 6 kb, more than 6.1 kb, more than 6.3 kb, or more than 6.5 kb. The AAV capsid can pack a nucleic acid that can be about 6.7 kb in length. The genetic cargo capacity of the variant AAV capsids can be, or can be about, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, or a number or a range between any two of these values. The genetic cargo capacity of the variant AAV capsids can be at least, or can be at most, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10.0 kb.

The genetic cargo capacity can be: (i) the maximum length of a single-stranded DNA molecule that the variant AAV capsid is capable of protecting from DNAse I digestion; and/or (ii) the maximum length of a double-stranded DNA molecule that the variant AAV capsid is capable of protecting from DNAse I digestion. The single-stranded DNA molecule can be capable of self-hybridizing to form a double-stranded region. The single-stranded DNA molecule can comprise a self-complementary AAV (scAAV) vector. The variant AAV capsid can protect the nucleic acid from DNAse I digestion. The variant AAV capsid can have at least 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 200%, 300%, 400%, 00%, 600%, 700%, 800%, 900%, or a 1000% of free DNase I protected titer of the corresponding parental AAV capsid.

The variant AAV capsid can be at least about 10%, at least about 20%, at least about 30%, at least about 40%, or at least about 50% larger in diameter and/or genetic cargo capacity as compared to the corresponding parental AAV capsid and/or a wildtype AAV capsid. The corresponding parental AAV capsid can comprise a genetic cargo capacity of less than about 4.8 kb, of less than about 4.9 kb, of less than about 5.0 kb, of less than about 5.1 kb, or of less than about 5.2 kb. The corresponding parental AAV capsid can comprise a diameter of less than about 25 nm, of less than about 26 nm, of less than about 27 nm, or of less than about 28 nm. The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) larger diameter and/or genetic cargo capacity as compared to the corresponding parental AAV capsid.

The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) greater molecular weight as compared to the corresponding parental AAV capsid.

The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) more capsid subunits as compared to the corresponding parental AAV capsid. The variant AAV capsid can comprise a triangulation number of 1, 2, 3, 4, or 5. The variant AAV capsid can comprise at least about 60 (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or a number or a range between any two of these values) subunits. Subunits can be monomeric or multimers.

The packaging efficiency of the variant AAV capsid can be at least about 0.1% (e.g., 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the packaging efficiency of the corresponding parental AAV capsid and/or a wildtype AAV capsid.

The transduction efficiency of the variant AAV capsid can be at least about 0.1% (e.g., 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the transduction efficiency of the corresponding parental AAV capsid and/or a wildtype AAV capsid.

The variant AAV capsid protein can comprise an amino acid sequence that can be at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence of any one of SEQ ID NOs: 1-108.

The reference AAV disclosed herein, in some cases, is AAV9 or AAV-DJ. However, the reference AAV can be any serotype, e.g. a serotype selected from AAV1, AAV2, AAV3, AAV4, AAV5, AAV6, AAV7, AAV8, AAV9, AAV10, AAV11, AAV12, or variant thereof. In many instances, the reference AAV is the parental AAV, e.g., the corresponding unmodified AAV from which the variant AAV was engineered.

The AAV capsid protein from which the engineered AAV capsid protein of the present disclosure is produced is referred to as a “parental” AAV capsid protein, or a “corresponding unmodified capsid protein”. The parental AAV capsid protein can have a serotype selected from AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

The variant AAV capsid and/or variant AAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein. In some instances, the second molecule is a detectable moiety. For example, the modified AAV capsid and/or rAAV capsid protein conjugated to a detectable moiety can be used for in vitro, ex vivo, or in vivo biomedical research applications, the detectable moiety used to visualize the modified capsid protein. The modified AAV capsid and/or rAAV capsid protein conjugated to a detectable moiety can also be used for diagnostic purposes.

rAAV, Heterologous Nucleic Acids, and Payloads

Disclosed herein include recombinant AAVs (rAAVs). In some embodiments, the rAAVs comprise: (a) a variant AAV capsid provided herein; and (b) a heterologous nucleic acid. The heterologous nucleic acid can comprise a polynucleotide encoding a payload. The payload can comprise a payload RNA agent and/or a payload protein.

Disclosed herein include populations of rAAV. Disclosed herein include populations of variant capsids. The average diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The average diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm. The average can be the mean, median or mode. The mean can be the arithmetic mean, geometric mean, and/or harmonic mean. The diameter can be calculated as the mean of the major axis length and the minor axis length. The diameter can be measured by transmission electron microscopy (TEM). The diameter can be hydrodynamic diameter, e.g., measured by dynamic light scattering (DLS).

The diameter of the viral capsids of the population of rAAV can vary (e.g., can range from about 20 nm to about 100 nm). For example, the diameter and/or average diameter of the viral capsids of the population of rAAV can range from about 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, or a number or a range between any two of these values. The minimum diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The minimum diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm.

The maximum diameter of the viral capsids of the population of rAAV can vary. The maximum diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The maximum diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm.

The heterologous nucleic acid can comprise a single-stranded DNA molecule, a double-stranded DNA molecule, a single-stranded RNA molecule, a double-stranded RNA molecule, or any combination thereof. The single-stranded DNA molecule can be capable of self-hybridizing to form a double-stranded region. The single-stranded DNA molecule can comprise a self-complementary AAV (scAAV) vector. The length of the heterologous nucleic acid can vary. The length of the heterologous nucleic acid can be, or can be about, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, or a number or a range between any two of these values. The length of the heterologous nucleic acid can be at least, or can be at most, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10.0 kb.

The length of the heterologous nucleic acid can be at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the genetic cargo capacity of the variant AAV capsid.

The heterologous nucleic acid can comprise a 5′ inverted terminal repeat (ITR) and a 3′ ITR. The payload can comprise a protein. The heterologous nucleic acid can comprise a promoter operably linked to the polynucleotide encoding a payload. The promoter can induce the transcription of the polynucleotide. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can bind the one or more silencer effector binding sequences, thereby reducing the stability of the payload transcript and/or reducing the translation of the payload transcript. The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The payload can comprise an RNA. The payload RNA agent can comprise one or more of dsRNA, siRNA, shRNA, pre-miRNA, pri-miRNA, miRNA, stRNA, lncRNA, piRNA, and snoRNA. The payload RNA agent can inhibit or suppress the expression of a gene of interest in a cell. In some embodiments, the gene of interest is SOD1, MAPT, APOE, HTT, C90RF72, TDP-43, APP, BACE, SNCA, ATXN1, ATXN2, ATXN3, ATXN7, SCN1A-SCN5A, or SCN8A-SCN11A. The heterologous nucleic acid can comprise a polynucleotide encoding one or more secondary proteins. The payload protein and the one or more secondary proteins can comprise a synthetic protein circuit. The heterologous nucleic acid can comprise a single-stranded AAV (ssAAV) vector or a self-complementary AAV (scAAV) vector.

The promoter can be, or can comprise, a ubiquitous promoter. The ubiquitous promoter can be, for example, a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, and any combination thereof.

The promoter can be an inducible promoter. The inducible promoter can be a tetracycline responsive promoter, a TRE promoter, a Tre3G promoter, an ecdysone responsive promoter, a cumate responsive promoter, a glucocorticoid responsive promoter, estrogen responsive promoter, a PPAR-γ promoter, or an RU-486 responsive promoter.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. The tissue specific promoter can be a neuron-specific promoter. The neuron-specific promoter can comprise a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be a muscle-specific promoter, e.g., a MCK promoter.

The promoter can comprise an intronic sequence. The promoter can comprise a bidirectional promoter and/or an enhancer. The enhancer can be a CMV enhancer. One or more cells of a subject can comprise an endogenous version of the payload. The promoter can comprise or be derived from the promoter of the endogenous version. One or more cells of a subject can comprise an endogenous version of the payload, e.g., a payload not truncated relative to the endogenous version.

In some embodiments, the promoter is less than 1 kb. Alternatively, the promoter can be greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 bp. The promoter can have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 bp. The promoter can provide expression of the therapeutic gene expression product for a period of time in targeted tissues such as, but not limited to, the central nervous system and peripheral organs (e.g., lung). Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload can be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

The payload protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or a combination thereof.

The payload protein can comprise a disease-associated protein. The level of expression of the disease-associated protein correlates with the occurrence and/or progression of the disease. The payload protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a nuclear localization signal (NLS) or a nuclear export signal (NES).

The payload protein can comprise a CRE recombinase, GCaMP, a cell therapy component, a knock-down gene therapy component, a cell-surface exposed epitope, or any combination thereof. The payload protein can comprise a chimeric antigen receptor. The payload protein can comprise a diagnostic agent. For example, the diagnostic agent can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

The payload protein can comprise a programmable nuclease. In some embodiments, the programmable nuclease is: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, dcas9-deaminase fusions; dcas9-Krab, dCas9-VP64, dCas9-Teti, dcas9-transcriptional regulator fusions; Dcas9-fluorescent protein fusions; Cas13-fluorescent protein fusions; RCas9-fluorescent protein fusions; and Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The heterologous nucleic acid further can comprise a polynucleotide encoding (i) a targeting molecule and/or (ii) a donor nucleic acid. The targeting molecule can associate with the programmable nuclease. The targeting molecule can comprise single strand DNA or single strand RNA. The targeting molecule can comprise a single guide RNA (sgRNA).

Disclosed herein are heterologous nucleic acids comprising a polynucleotide encoding one or more payload genes. The rAAV provided herein can comprise one or more of the heterologous nucleic acids disclosed herein. The heterologous nucleic acid can comprise a polynucleotide encoding a payload (e.g., a payload gene). The payload gene can encode a payload RNA agent and/or payload protein. The heterologous nucleic acid can comprise a promoter operably linked to the polynucleotide encoding a payload. As disclosed herein, the payload gene is operatively linked with appropriate regulatory elements. The one or more payload genes of the heterologous nucleic acid can comprise a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. The one or more payload genes of the heterologous nucleic acid can comprise one or more synthetic protein circuit components. The one or more payload genes of the heterologous nucleic acid can comprise can entire synthetic protein circuit comprising one or more synthetic protein circuit components. The one or more payload genes of the heterologous nucleic acid can comprise two or more synthetic protein circuits.

The payload protein can be any protein, including naturally-occurring and non-naturally occurring proteins. Examples of payload protein include, but are not limited to, luciferases; fluorescent proteins (e.g., GFP); growth hormones (GHs); insulin-like growth factors (IGFs); granulocyte colony-stimulating factors (G-CSFs); erythropoietin (EPO); insulin, such as proinsulin, preproinsulin, insulin, insulin analogs, and the like; antibodies, such as hybrid antibodies, chimeric antibodies, humanized antibodies, monoclonal antibodies; antigen binding fragments of an antibody (Fab fragments), single-chain variable fragments of an antibody (scFV fragments); dystrophin; clotting factors; cystic fibrosis transmembrane conductance regulator (CFTR); interferons, and variants of any of the above proteins.

The payload protein can be a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and variants thereof.

The payload protein can be an active fragment of a protein, such as any of the aforementioned proteins. The payload protein is a fusion protein comprising some or all of two or more proteins. A fusion protein can comprise all or a portion of any of the aforementioned proteins. The payload protein can be a multi-subunit protein. For example, the payload protein can comprise two or more subunits, or two or more independent polypeptide chains. The payload protein can be an antibody. Examples of antibodies include, but are not limited to, antibodies of various isotypes (e.g., IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. The antibody can be a full-length antibody.

The payload gene can encode a pro-survival protein (e.g., Bcl-2, Bcl-XL, Mcl-1 and A1). The payload gene can encode a apoptotic factor or apoptosis-related protein such as, AIF, Apaf (e.g., Apaf-1, Apaf-2, and Apaf-3), oder APO-2 (L), APO-3 (L), Apopain, Bad, Bak, Bax, Bcl-2, Bcl-xL, Bcl-xs, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, or transglutaminase.

In some embodiments, the payload gene encodes a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. The payload gene can encode a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or a combination thereof.

In some embodiments, the payload gene encodes a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

In some embodiments, the payload gene encodes immunogenic material capable of stimulating an immune response (e.g., an adaptive immune response) such as, for example, antigenic peptides or proteins from a pathogen. The expression of the antigen can stimulate the body's adaptive immune system to provide an adaptive immune response. Thus, it is contemplated that the heterologous nucleic acids provided herein can be employed as vaccines for the prophylaxis or treatment of infectious diseases (e.g., as vaccines).

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload gene expression in the target cell can vary. For example, The payload gene can encode a payload protein. The amount of the payload protein expressed in the subject (e.g., the serum of the subject) can vary. For example, The protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. The payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein encoded by a payload gene can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. The payload protein is at least about 480 amino acids in length. The payload protein is at least about 500 amino acids in length. The payload protein is about 750 amino acids in length.

The payload genes can have different lengths in different implementations. The number of payload genes can vary. The number of payload genes in a heterologous nucleic acid can be, or can be about, 1, 2, 3, 4, 5, 6, 7, 8, 9, 10, 11, 12, 13, 14, 15, 16, 17, 18, 19, 20, 21, 22, 23, 24, 25, or a number or a range between any two of these values. The number of payload genes in a heterologous nucleic acid can be at least, or can be at most, 1, 2, 3, 4, 5, 6, 7, 8, 9, 10, 11, 12, 13, 14, 15, 16, 17, 18, 19, 20, 21, 22, 23, 24, 25. A payload genes is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length. A payload gene is at least, or is at most, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000 nucleotides in length.

The payload can be an inducer of cell death, for example by a non-endogenous cell death pathway (e.g., a bacterial pore-forming toxin). The payload can be a pro-survival protein. The payload is a modulator of the immune system. The payload can activate an adaptive immune response, and innate immune response, or both. The payload gene encodes immunogenic material capable of stimulating an immune response (e.g., an adaptive immune response) such as, for example, antigenic peptides or proteins from a pathogen. The expression of the antigen can stimulate the body's adaptive immune system to provide an adaptive immune response. Thus, it is contemplated that the compositions provided herein can be employed as vaccines for the prophylaxis or treatment of infectious diseases (e.g., as vaccines). The payload protein can comprise a CRE recombinase, GCaMP, a cell therapy component, a knock-down gene therapy component, a cell-surface exposed epitope, or any combination thereof. The payload comprises CFTR, GDE, OTOF, DYSF, MYO7A, ABCA4, F8, CEP290, CDH23, DMD, and ALMS1.

A payload can comprise a non-protein coding gene, such as a payload RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. Then, a non-protein coding gene can also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. a non-protein coding gene. For example, the RNAs described herein can be used to inhibit gene expression in a target cell, for example, a cell in the central nervous system (CNS) or peripheral organ (e.g., lung). In some cases, inhibition of gene expression refers to an inhibition by at least about 20%, 30%, 40%, 50%, 60%, 70%, 80%, 85%, 90%, 95% and 100%. In some cases, the protein product of the targeted gene can be inhibited by at least about 20%, 30%, 40%, 50%, 60%, 70%, 80%, 85%, 90%, 95% and 100%. The gene can be either a wild type gene or a gene with at least one mutation. The targeted protein can be either a wild type protein or a protein with at least one mutation.

Examples of payload genes include a sequence associated with a signaling biochemical pathway, e.g., a signaling biochemical pathway-associated gene or polynucleotide (e.g., a signal transducer). The methods and compositions disclosed herein comprise knockdown of an endogenous signal transducer accompanied by tuned expression of a payload protein comprising an appropriate version of signal transducer. Examples of payloads contemplated herein include a disease-associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It can be a gene that becomes expressed at an abnormally high level; it can be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products can be known or unknown, and can be at a normal or abnormal level. Signal transducers can be associated with one or more diseases or disorders. A disease or disorder can be characterized by an aberrant signaling of one or more signal transducers disclosed herein. The activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 1-3. The methods and compositions disclosed herein can prevent or treat one or more of the diseases and disorders listed in Tables 1-3. The payload can comprise a replacement version of the signal transducer. The methods and compositions further comprise knockdown of the corresponding endogenous signal transducer. The payload can comprise the product of a gene listed in listed in Tables 1-3. The payload can ameliorate a disease or disorder characterized by an aberrant signaling of one or more signaling transducers. The payload can diminish the activation level of one or more signal transducers (e.g., signal transducers with aberrant overactive signaling, signal transducers listed in Tables 1-3). The payload can increase the activation level of one or more signal transducers (e.g., signal transducers with aberrant underactive signaling). The payload can modulate the abundance, location, stability, and/or activity of activators or repressors of said signal transducers.

TABLE 1
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; Notch2; Notch3; |
| | Notch4; AKT; AKT2; AKT3; HIF; HIF1a; HIF3a; Met; HRG; Bcl2; PPAR alpha; |
| | PPAR gamma; WT1 (Wilms Tumor); FGF Receptor Family members (5 |
| | members: 1, 2, 3, 4, 5); CDKN2a; APC; RB (retinoblastoma); MEN1; VHL; |
| | BRCA1; BRCA2; AR (Androgen Receptor); TSG101; IGF; IGF Receptor; Igf1 (4 |
| | variants); Igf2 (3 variants); Igf 1 Receptor; Igf 2 Receptor; Bax; Bcl2; caspases |
| | family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 (Cplx1); Tph1 |
| | Tryptophan hydroxylase; Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; DAOA; DTNBP1; Dao (Dao1) |
| Trinucleotide Repeat | HTT (Huntington's Dx); SBMA/SMAX1/AR (Kennedy's Dx); FXN/X25 |
| Disorders | (Friedrich's Ataxia); ATX3 (Machado- Joseph's Dx); ATXN1 and ATXN2 |
| | (spinocerebellar ataxias); DMPK (myotonic dystrophy); Atrophin-1 and Atn1 |
| | (DRPLA Dx); CBP (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related disorders | Prp |
| ALS | SOD1; ALS2; STEX; FUS; TARDBP; VEGF (VEGF-a; VEGF-b; VEGF-c) |
| Drug addiction | Prkce (alcohol); Drd2; Drd4; ABAT (alcohol); GRIA2; Grm5; Grin1; Htr1b; |
| | Grin2a; Drd3; Pdyn; Gria1 (alcohol) |
| Autism | Mecp2; BZRAP1; MDGA2; Sema5A; Neurexin 1; Fragile X (FMR2 (AFF2); |
| | FXR1; FXR2; Mglur5) |
| Alzheimer's Disease | E1; CHIP; UCH; UBB; Tau; LRP; PICALM; Clusterin; PS1; SORL1; CR1; |
| | Vldlr; Uba1; Uba3; CHIP28 (Aqp1, Aquaporin 1); Uchl1; Uchl3; APP |
| Inflammation | IL-10; IL-1 (IL-1a; IL-1b); IL-13; IL-17 (IL-17a (CTLA8); IL- 17b; IL-17c; IL- |
| | 17d; IL-17f); II-23; Cx3cr1; ptpn22; TNFa; NOD2/CARD15 for IBD; IL-6; IL-12 |
| | (IL-12a; IL-12b); CTLA4; Cx3cl1 |
| Parkinson's Disease | x-Synuclein; DJ-1; LRRK2; Parkin; PINK1 |

TABLE 2
SIGNAL TRANSDUCERS

| | |
|---|---|
| Blood and | Anemia (CDAN1, CDA1, RPS19, DBA, PKLR, PK1, NT5C3, UMPH1, PSN1, RHAG, |
| coagulation | RH50A, NRAMP2, SPTB, ALAS2, ANH1, ASB, ABCB7, ABC7, ASAT); Bare |
| diseases and | lymphocyte syndrome (TAPBP, TPSN, TAP2, ABCB3, PSF2, RING11, MHC2TA, |
| disorders | C2TA, RFX5, RFXAP, RFX5); Bleeding disorders (TBXA2R, P2RX1, P2X1); Factor |
| | H and factor H-like 1 (HF1, CFH, HUS); Factor V and factor VIII (MCFD2); Factor |
| | VII deficiency (F7); Factor X deficiency (F10); Factor XI deficiency (F11); Factor XII |
| | deficiency (F12, HAF); Factor XIIIA deficiency (F13A1, F13A); Factor XIIIB |
| | deficiency (F13B); Fanconi anemia (FANCA, FACA, FA1, FA, FAA, FAAP95, |
| | FAAP90, FLJ34064, FANCB, FANCC, FACC, BRCA2, FANCD1, FANCD2, FANCD, |
| | FACD, FAD, FANCE, FACE, FANCF, XRCC9, FANCG, BRIP1, BACH1, FANCJ, |
| | PHF9, FANCL, FANCM, KIAA1596); Hemophagocytic lymphohistiocytosis disorders |
| | (PRF1, HPLH2, UNC13D, MUNC13-4, HPLH3, HLH3, FHL3); Hemophilia A (F8, |
| | F8C, HEMA); Hemophilia B (F9, HEMB), Hemorrhagic disorders (PI, ATT, F5); |
| | Leukocyde deficiencies and disorders (ITGB2, CD18, LCAMB, LAD, EIF2B1, |
| | EIF2BA, EIF2B2, EIF2B3, EIF2B5, LVWM, CACH, CLE, EIF2B4); Sickle cell anemia |
| | (HBB); Thalassemia (HBA2, HBB, HBD, LCRB, HBA1). |
| Cell | B-cell non-Hodgkin lymphoma (BCL7A, BCL7); Leukemia (TAL1 TCL5, SCL, TAL2, |
| dysregulation | FLT3, NBS1, NBS, ZNFN1A1, IK1, LYF1, HOXD4, HOX4B, BCR, CML, PHL, ALL, |
| and oncology | ARNT, KRAS2, RASK2, GMPS, AF10, ARHGEF12, LARG, KIAA0382, CALM, |
| diseases and | CLTH, CEBPA, CEBP, CHIC2, BTL, FLT3, KIT, PBT, LPP, NPM1, NUP214, D9S46E, |
| disorders | CAN, CAIN, RUNX1, CBFA2, AML1, WHSC1L1, NSD3, FLT3, AF1Q, NPM1, |
| | NUMA1, ZNF145, PLZF, PML, MYL, STAT5B, AF10, CALM, CLTH, ARL11, |
| | ARLTS1, P2RX7, P2X7, BCR, CML, PHL, ALL, GRAF, NF1, VRNF, WSS, NFNS, |
| | PTPN11, PTP2C, SHP2, NS1, BCL2, CCND1, PRAD1, BCL1, TCRA, GATA1, GF1, |
| | ERYF1, NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, SDF1); |
| and immune | Autoimmune lymphoproliferative syndrome (TNFRSF6, APT1, FAS, CD95, |
| related | ALPS1A); Combined immunodeficiency, (IL2RG, SCIDX1, SCIDX, IMD4); HIV-1 |
| diseases and | (CCL5, SCYA5, D17S136E, TCP228), HIV susceptibility or infection (IL10, CSIF, |
| disorders | CMKBR2, CCR2, CMKBR5, CCCKR5 (CCR5)); Immunodeficiencies (CD3E, CD3G, |
| | AICDA, AID, HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); Inflammation |
| | (IL-10, IL-1 (IL-1a, IL-1b), IL-13, IL-17 (IL-17a (CTLA8), IL-17b, IL-17c, IL-17d, IL- |
| | 17f), II-23, Cx3cr1, ptpn22, TNFa, NOD2/CARD15 for IBD, IL-6, IL-12 (IL-12a, IL- |
| | 12b), CTLA4, Cx3cl1); Severe combined immunodeficiencies (SCIDs)(JAK3, JAKL, |
| | DCLRE1C, ARTEMIS, SCIDA, RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis (APOA1, APP, AAA, CVAP, AD1, |
| liver, kidney | GSN, FGA, LYZ, TTR, PALB); Cirrhosis (KRT18, KRT8, CIRH1A, NAIC, TEX292, |
| and protein | KIAA1988); Cystic fibrosis (CFTR, ABCC7, CF, MRP7); Glycogen storage diseases |
| diseases and | (SLC2A2, GLUT2, G6PC, G6PT, G6PT1, GAA, LAMP2, LAMPB, AGL, GDE, GBE1, |
| disorders | GYS2, PYGL, PFKM); Hepatic adenoma (TCF1, HNF1A, MODY3), Hepatic failure, |
| | early onset, and neurologic disorder (SCOD1, SCO1), Hepatic lipase deficiency |
| | (LIPC), Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, CASP8, |
| | MCH5); Medullary cystic kidney disease (UMOD, HNFJ, FJHN, MCKD2, |
| | ADMCKD2); Phenylketonuria (PAH, PKU1, QDPR, DHPR, PTS); Polycystic kidney |
| | and hepatic disease (FCYT, PKHD1, ARPKD, PKD1, PKD2, PKD4, PKDTS, PRKCSH, |
| | G19P1, PCLD, SEC63). |
| Muscular/ | Becker muscular dystrophy (DMD, BMD, MYF6), Duchenne Muscular Dystrophy |
| Skeletal | (DMD, BMD); Emery-Dreifuss muscular dystrophy (LMNA, LMN1, EMD2, FPLD, |
| diseases and | CMD1A, HGPS, LGMD1B, LMNA, LMN1, EMD2, FPLD, CMD1A); |
| disorders | Facioscapulohumeral muscular dystrophy (FSHMD1A, FSHD1A); Muscular |
| | dystrophy (FKRP, MDC1C, LGMD2I, LAMA2, LAMM, LARGE, KIAA0609, MDC1D, |
| | FCMD, TTID, MYOT, CAPN3, CANP3, DYSF, LGMD2B, SGCG, LGMD2C, DMDA1, |
| | SCG3, SGCA, ADL, DAG2, LGMD2D, DMDA2, SGCB, LGMD2E, SGCD, SGD, |
| | LGMD2F, CMD1L, TCAP, LGMD2G, CMD1N, TRIM32, HT2A, LGMD2H, FKRP, |
| | MDC1C, LGMD2I, TTN, CMD1G, TMD, LGMD2J, POMT1, CAV3, LGMD1C, |
| | SEPN1, SELN, RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, TIRC7, OC116, |
| | OPTB1); Muscular atrophy (VAPB, VAPC, ALS8, SMN1, SMA1, SMA2, SMA3, |
| | SMA4, BSCL2, SPG17, GARS, SMAD1, CMT2D, HEXB, IGHMBP2, SMUBP2, |
| | CATF1, SMARD1). |
| Neurological | ALS (SOD1, ALS2, STEX, FUS, TARDBP, VEGF (VEGF-a, VEGF-b, VEGF-c); |
| and neuronal | Alzheimer disease (APP, AAA, CVAP, AD1, APOE, AD2, PSEN2, AD4, STM2, |
| diseases and | APBB2, FE65L1, NOS3, PLAU, URK, ACE, DCP1, ACE1, MPO, PACIP1, PAXIP1L, |
| disorders | PTIP, A2M, BLMH, BMH, PSEN1, AD3); Autism (Mecp2, BZRAP1, MDGA2, |
| | Sema5A, Neurexin 1, GLO1, MECP2, RTT, PPMX, MRX16, MRX79, NLGN3, NLGN4, |
| | KIAA1260, AUTSX2); Fragile X Syndrome (FMR2, FXR1, FXR2, mGLUR5); |
| | Huntington's disease and disease like disorders (HD, IT15, PRNP, PRIP, JPH3, JP3, |
| | HDL2, TBP, SCA17); Parkinson disease (NR4A2, NURR1, NOT, TINUR, SNCAIP, |
| | TBP, SCA17, SNCA, NACP, PARK1, PARK4, DJ1, PARK7, LRRK2, PARK8, PINK1, |
| | PARK6, UCHL1, PARK5, SNCA, NACP, PARK1, PARK4, PRKN, PARK2, PDJ, DBH, |
| | NDUFV2); Rett syndrome (MECP2, RTT, PPMX, MRX16, MRX79, CDKL5, STK9, |
| | MECP2, RTT, PPMX, MRX16, MRX79, x-Synuclein, DJ-1); Schizophrenia |
| | (Neuregulin1 (Nrg1), Erb4 (receptor for Neuregulin), Complexin1 (Cplx1), Tph1 |
| | Tryptophan hydroxylase, Tph2, Tryptophan hydroxylase 2, Neurexin 1, GSK3, GSK3a, |
| | GSK3b, 5-HTT (Slc6a4), COMT, DRD (Drd1a), SLC6A3, DAOA, DTNBP1, Dao |
| | (Dao1)); Secretase Related Disorders (APH-1 (alpha and beta), Presenilin (Psen1), |
| | nicastrin, (Ncstn), PEN-2, Nos1, Parp1, Nat1, Nat2); Trinucleotide Repeat Disorders |
| | (HTT (Huntington's Dx), SBMA/SMAX1/AR (Kennedy's Dx), FXN/X25 (Friedrich's |
| | Ataxia), ATX3 (Machado- Joseph's Dx), ATXN1 and ATXN2 (spinocerebellar ataxias), |
| | DMPK (myotonic dystrophy), Atrophin-1 and Atn1 (DRPLA Dx), CBP (Creb-BP - global |
| | instability), VLDLR (Alzheimer's), Atxn7, Atxn10). |
| Ocular | Age-related macular degeneration (Abcr, Ccl2, Cc2, cp (ceruloplasmin), Timp3, |
| diseases and | cathepsinD, Vldlr, Ccr2); Cataract (CRYAA, CRYA1, CRYBB2, CRYB2, PITX3, |
| disorders | BFSP2, CP49, CP47, CRYAA, CRYA1, PAX6, AN2, MGDA, CRYBA1, CRYB1, |
| | CRYGC, CRYG3, CCL, LIM2, MP19, CRYGD, CRYG4, BFSP2, CP49, CP47, HSF4, |
| | CTM, HSF4, CTM, MIP, AQP0, CRYAB, CRYA2, CTPP2, CRYBB1, CRYGD, |
| | CRYG4, CRYBB2, CRYB2, CRYGC, CRYG3, CCL, CRYAA, CRYA1, GJA8, CX50, |
| | CAE1, GJA3, CX46, CZP3, CAE3, CCM1, CAM, KRIT1); Corneal clouding and |
| | dystrophy (APOA1, TGFBI, CSD2, CDGG1, CSD, BIGH3, CDG2, TACSTD2, TROP2, |
| | M1S1, VSX1, RINX, PPCD, PPD, KTCN, COL8A2, FECD, PPCD2, PIP5K3, CFD); |
| | Cornea plana congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, NTG, NPG, |
| | CYP1B1, GLC3A); Leber congenital amaurosis (CRB1, RP12, CRX, CORD2, CRD, |
| | RPGRIP1, LCA6, CORD9, RPE65, RP20, AIPL1, LCA4, GUCY2D, GUC2D, LCA1, |
| | CORD6, RDH12, LCA3); Macular dystrophy (ELOVL4, ADMD, STGD2, STGD3, |
| | RDS, RP7, PRPH2, PRPH, AVMD, AOFMD, VMD2). |

TABLE 3
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; MAPK9; |
| | CDK2; PPP2CA; PIM1; ITGB7; YWHAZ; ILK; TP53; RAF1; IKBKG; RELB; |
| | DYRK1A; CDKN1A; ITGB1; MAP2K2; JAK1; AKT1; JAK2; PIK3R1; |
| | CHUK; PDPK1; PPP2R5C; CTNNB1; MAP2K1; NFKB1; PAK3; ITGB3; |
| | CCND1; GSK3A; FRAP1; SFN; ITGA2; TTK; CSNK1A1; BRAF; GSK3B; |
| | AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid | RAC1; TAF4B; EP300; SMAD2; TRAF6; PCAF; ELK1; MAPK1; SMAD3; |
| Receptor Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; RAC1; RAP1A; |
| Signaling | E1F4E; PRKCZ; NRP1; NTRK2; ARHGEF7; SMO; ROCK2; MAPK1; PGF; |
| | RAC2; PTPN11; GNAS; AKT2; PIK3CA; ERBB2; PRKCI; PTK2; CFL1; |
| | GNAQ; PIK3CB; CXCL12; PIK3C3; WNT11; PRKD1; GNB2L1; ABL1; |
| | MAPK3; ITGA1; KRAS; RHOA; PRKCD; PIK3C2A; ITGB7; GLI2; PXN; |
| | VASP; RAF1; FYN; ITGB1; MAP2K2; PAK4; ADAM17; AKT1; PIK3R1; |
| | GLI1; WNT5A; ADAM10; MAP2K1; PAK3; ITGB3; CDC42; VEGFA; |
| | ITGA2; EPHA8; CRKL; RND1; GSK3B; AKT3; PRKCA |
| Ephrin Receptor | PRKCE; ITGAM; ROCK1; ITGA5; CXCR4; IRAK1; PRKAA2; EIF2AK2; |
| Signaling | RAC1; RAP1A; GRK6; ROCK2; MAPK1; PGF; RAC2; PTPN11; GNAS; |
| | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRK1A; ITGB1; MAP2K2; PAK4, AKT1; JAK2; STAT3; ADAM10; |
| | MAP2K1; PAK3; ITGB3; CDC42; VEGFA; ITGA2; EPHA8; TTK; CSNK1A1; |
| | CRKL; BRAF; PTPN13; ATF4; AKT3; SGK |
| Actin Cytoskeleton | ACTN4; PRKCE; ITGAM; ROCK1; ITGA5; IRAK1; PRKAA2; EIF2AK2; |
| Signaling | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; MAPK9; |
| | CDK2; PIM1; PIK3C2A; ITGB7; PPP1CC; PXN; VIL2; RAF1; GSN; |
| | DYRK1A; ITGB1; MAP2K2; PAK4; PIP5K1A; PIK3R1; MAP2K1; PAK3; |
| | ITGB3; CDC42; APC; ITGA2; TTK; CSNK1A1; CRKL; BRAF; VAV3; SGK |
| Huntington's Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKC1; |
| | HSPA5; REST; GNAQ; PIK3CB; PIK3C3; MAPK8; IGF1R; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; ATF4; |
| | AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; PTPN11; AKT2; IKBKB; |
| Signaling | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; MAP3K7; |
| | MAP2K2; AKT1; PIK3R1; CHUK; MAP2K1; NFKB1; CDC42; GSK3A; |
| | FRAP1; BCL6; BCL10; JUN; GSK3B; ATF4; AKT3; VAV3; RPS6KB1 |
| Leukocyte | ACTN4; CD44; PRKCE; ITGAM; ROCK1; CXCR4; CYBA; RAC1; RAP1A; |
| Extravasation Signaling | PRKCZ; ROCK2; RAC2; PTPN11; MMP14; PIK3CA; PRKCI; PTK2; |
| | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; NOX1; |
| | PXN; VIL2; VASP; ITGB1; MAP2K2; CTNND1; PIK3R1; CTNNB1; CLDN1; |
| | CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; ARHGEF7; |
| | MAPK1; RAC2; CAPNS1; AKT2; CAPN2; PIK3CA; PTK2; PIK3CB; PIK3C3; |
| | MAPK8; CAV1; CAPN1; ABL1; MAPK3; ITGA1; KRAS; RHOA; SRC; |
| | PIK3C2A; ITGB7; PPP1CC; ILK; PXN; VASP; RAF1; FYN; ITGB1; |
| | MAP2K2; PAK4; AKT1; PIK3R1; TNK2; MAP2K1; PAK3; ITGB3; CDC42; |
| | RND3; ITGA2; CRKL; BRAF; GSK3B; AKT3 |
| Acute Phase Response | IRAK1; SOD2; MYD88; TRAF6; ELK1; MAPK1; PTPN11; AKT2; IKBKB; |
| Signaling | PIK3CA; FOS; NFKB2; MAP3K14; PIK3CB; MAPK8; RIPK1; MAPK3; |
| | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; MAP3K7; |
| | MAP2K2; AKT1; JAK2; PIK3R1; CHUK; STAT3; MAP2K1; NFKB1; FRAP1; |
| | CEBPB; JUN; AKT3; IL1R1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| | PIK3CA; CHEK1; TP53INP1; BCL2; PIK3CB; PIK3C3; MAPK8; THBS1; |
| | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAF1; CTNNB1; SIRT1; CCND1; PRKDC; ATM; SFN; |
| | CDKN2A; JUN; SNAI2; GSK3B; BAχ; AKT3 |
| Aryl Hydrocarbon | HSPB1; EP300; FASN; TGM2; RXRA; MAPK1; NQO1; NCOR2; SP1; ARNT; |
| Receptor Signaling | CDKN1B; FOS; CHEK1; SMARCA4; NFKB2; MAPK8; ALDH1A1; ATR; |
| | E2F1; MAPK3; NRIP1; CHEK2; RELA; TP73; GSTP1; RB1; SRC; CDK2; |
| | AHR; NFE2L2; NCOA3; TP53; TNF; CDKN1A; NCOA2; APAF1; NFKB1; |
| | CCND1; ATM; ESR1; CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; NCOR2; PIK3CA; ARNT; |
| Signaling | PRKCI; NFKB2; CAMK2A; PIK3CB; PPP2R1A; PIK3C3; MAPK8; PRKD1; |
| | ALDH1A1; MAPK3; NRIP1; KRAS; MAPK13; PRKCD; GSTP1; MAPK9; |
| | NOS2A; ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK Signaling | PRKCE; IRAK1; PRKAA2; EIF2AK2; RAC1; ELK1; GRK6; MAPK1; |
| | GADD45A; RAC2; PLK1; AKT2; PIK3CA; FADD; CDK8; PIK3CB; PIK3C3; |
| | MAPK8; RIPK1; GNB2L1; IRS1; MAPK3; MAPK10; DAXX; KRAS; |
| | PRKCD; PRKAA1; MAPK9; CDK2; PIM1; PIK3C2A; TRAF2; TP53; LCK; |
| | MAP3K7; DYRK1A; MAP2K2; PIK3R1; MAP2K1; PAK3; CDC42; JUN; |
| | TTK; CSNK1A1; CRKL; BRAF; SGK |
| PPAr/RXR Signaling | PRKAA2; EP300; INS; SMAD2; TRAF6; PPARA; FASN; RXRA; MAPK1; |
| | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; IL1R1; |
| | PRKCA; IL6; HSP90AA1; ADIPOQ |
| NF-KB Signaling | IRAKI; EIF2AK2; EP300; INS; MYD88; PRKCZ: TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4: |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin Signaling | ERBB4; PRKCE; ITGAM; ITGA5: PTEN; PRKCZ; ELK1; MAPK1; PTPN11; |
| | AKT2; EGFR; ERBB2; PRKCI; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPK1; MAP2K1; ITGB3; EREG; FRAP1; |
| | PSEN1; ITGA2; MYC; NRG1; CRKL; AKT3; PRKCA; HSP90AA1; RPS6KB1 |
| Wnt & Beta catenin | CD44; EP300; LRP6; DVL3; CSNK1E; GJA1; SMO; AKT2; PIN1; CDH1; |
| Signaling | BTRC; GNAQ; MARK2; PPP2R1A; WNT11; SRC; DKK1; PPP2CA; SOX6; |
| | SFRP2: ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin Receptor | PTEN; INS; EIF4E; PTPN1; PRKCZ; MAPK1; TSC1; PTPN11; AKT2; CBL; |
| Signaling | PIK3CA; PRKCI; PIK3CB; PIK3C3; MAPK8; IRS1; MAPK3; TSC2; KRAS; |
| | EIF4EBP1; SLC2A4; PIK3C2A; PPP1CC; INSR; RAF1; FYN; MAP2K2; |
| | JAK1; AKT1; JAK2; PIK3R1; PDPK1; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2: MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; RXRA; IKBKB; |
| | PRKCI; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; IL8; |
| | CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKCI; |
| | PTK2; FOS; PIK3CB; PIK3C3; MAPK8; IGF1R; IRS1; MAPK3; IGFBP7; |
| | KRAS; PIK3C2A; YWHAZ; PXN; RAF1; CASP9; MAP2K2; AKT1; PIK3R1; |
| | PDPK1; MAP2K1; IGFBP2; SFN; JUN; CYR61; AKT3; FOXO1; SRF; CTGF; |
| | RPS6KB1 |
| NRF2-mediated | PRKCE; EP300; SOD2; PRKCZ; MAPK1; SQSTM1; NQO1; PIK3CA; PRKCI; |
| Oxidative Stress | FOS; PIK3CB; PIK3C3; MAPK8; PRKD1; MAPK3; KRAS; PRKCD; GSTP1; |
| Response | MAPK9; FTL; NFE2L2; PIK3C2A; MAPK14; RAF1; MAP3K7; CREBBP; |
| | MAP2K2; AKT1; PIK3R1; MAP2K1; PPIB; JUN; KEAP1; GSK3B; ATF4; |
| | PRKCA; EIF2AK3; HSP90AA1 |
| Hepatic | EDN1; IGF1; KDR; FLT1; SMAD2; FGFR1; MET; PGF; SMAD3; EGFR; |
| Fibrosis/Hepatic | FAS; CSF1; NFKB2; BCL2; MYH9; IGF1R; IL6R; RELA; TLR4; PDGFRB; |
| Stellate Cell Activation | TNF; RELB; IL8; PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PPARGC1A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI Signaling | PRKCE; RAC1; PRKCZ; LYN; MAPK1; RAC2; PTPN11; AKT2; PIK3CA; |
| | SYK; PRKCI; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled | PRKCE; RAP1A; RGS16; MAPK1; GNAS; AKT2; IKBKB; PIK3CA; CREB1; |
| Receptor Signaling | GNAQ; NFKB2; CAMK2A; PIK3CB; PIK3C3; MAPK3; KRAS; RELA; SRC; |
| | PIK3C2A; RAF1; IKBKG; RELB; FYN; MAP2K2; AKT1; PIK3R1; CHUK; |
| | PDPK1; STAT3; MAP2K1; NFKB1; BRAF; ATF4; AKT3; PRKCA |
| Inositol Phosphate | PRKCE; IRAK1; PRKAA2; EIF2AK2; PTEN; GRK6; MAPK1; PLK1; AKT2; |
| Metabolism | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; MAPK8; |
| | CAV1; ABL1; MAPK3; KRAS; SRC; PIK3C2A; PDGFRB; RAF1; MAP2K2; |
| | JAK1; JAK2; PIK3R1; PDGFRA; STAT3; SPHK1; MAP2K1; MYC; JUN; |
| | CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer Cell | PRKCE; RAC1; PRKCZ; MAPK1; RAC2; PTPN11; KIR2DL3; AKT2; |
| Signaling | PIK3CA; SYK; PRKCI; PIK3CB; PIK3C3; PRKD1; MAPK3; KRAS; PRKCD; |
| | PTPN6; PIK3C2A; LCK; RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; CDKN1B; BTRC; ATR; ABL1; E2F1; |
| Checkpoint Regulation | HDAC2; HDAC7A; RB1; HDAC11; HDAC9; CDK2; E2F2; HDAC3; TP53; |
| | CDKN1A; CCND1; E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; NFKB2; PIK3CB; |
| Signaling | PIK3C3; MAPK8; MAPK3; KRAS; RELA, PIK3C2A; BTK; LCK; RAF1; |
| | IKBKG; RELB, FYN; MAP2K2; PIK3R1; CHUK; MAP2K1; NFKB1; ITK; |
| | BCL10; JUN; VAV3 |
| Death Receptor | CRADD; HSPB1; BID; BIRC4; TBK1; IKBKB; FADD; FAS; NFKB2; BCL2; |
| Signaling | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3; BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; PIM1; |
| | PIK3C2A; RAF1; MAP2K2; AKT1; JAK2; PIK3R1; STAT3; MAP2K1; |
| | CCND1; AKT3; STAT1 |
| Amyotrophic Lateral | BID; IGF1; RAC1; BIRC4; PGF; CAPNS1; CAPN2; PIK3CA; BCL2; PIK3CB; |
| Sclerosis Signaling | PIK3C3; BCL2L1; CAPN1; PIK3C2A; TP53; CASP9; PIK3R1; RAB5A; |
| | CASP1; APAF1; VEGFA; BIRC2; BAχ; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STAT5A; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; PLK1; AKT2; CDK8; |
| Nicotinamide | MAPK8; MAPK3; PRKCD; PRKAA1; PBEF1; MAPK9; CDK2; PIM1; |
| Metabolism | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPP1CC; MAPK14; |
| | NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; RAF1; |
| | MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKCI; GNAQ; |
| Depression | PPP2R1A; IGF1R; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor | TAF4B; EP300; CARM1; PCAF; MAPK1; NCOR2; SMARCA4; MAPK3; |
| Signaling | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein Ubiquitination | TRAF6; SMURF1; BIRC4; BRCA1; UCHL1; NEDD4; CBL; UBE2I; BTRC; |
| Pathway | HSPA5; USP7; USP10; FBXW7; USP9X; STUB1; USP22; B2M; BIRC2; |
| | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKCI; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor | IRAKI; EIF2AK2; MYD88; TRAF6; PPARA; ELK1; IKBKB; FOS; NFKB2; |
| Signaling | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; IL1R1; SRF; STAT1 |
| Neurotrophin/TRK | NTRK2; MAPK1; PTPN11; PIK3CA; CREB1; FOS; PIK3CB; PIK3C3; |
| Signaling | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKCI; GNAQ; |
| Potentiation | CAMK2A; PRKD1; MAPK3; KRAS; PRKCD; PPP1CC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in | EDN1; PTEN; EP300; NQO1; UBE2I; CREB1; ARNT; HIF1A; SLC2A4; |
| the Cardiovascular | NOS3; TP53; LDHA; AKT1; ATM; VEGFA; JUN; ATF4; VHL; HSP90AA1 |
| System | |
| LPS/IL-1 Mediated | IRAK1; MYD88; TRAF6; PPARA; RXRA; ABCA1, MAPK8; ALDH1A1; |
| Inhibition of RXR | GSTP1; MAPK9; ABCB1; TRAF2; TLR4; TNF; MAP3K7; NR1H2; SREBF1; |
| Function | JUN; IL1R1 |
| LXR/RXR Activation | FASN; RXRA; NCOR2; ABCA1; NFKB2; IRF3; RELA; NOS2A; TLR4; TNF; |
| | RELB; LDLR; NR1H2; NFKB1; SREBF1; IL1R1; CCL2; IL6; MMP9 |
| Amyloid Processing | PRKCE; CSNK1E; MAPK1; CAPNS1; AKT2; CAPN2; CAPN1; MAPK3; |
| | MAPK13; MAPT; MAPK14; AKT1; PSEN1; CSNK1A1; GSK3B; AKT3; APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling | KDR; FLT1; PGF; AKT2; PIK3CA; PIK3CB; PIK3C3; CAV1; PRKCD; NOS3; |
| in the Cardiovascular | PIK3C2A; AKT1; PIK3R1; VEGFA; AKT3; HSP90AA1 |
| System | |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated | RAP1A; MAPK1; GNAS; CREB1; CAMK2A; MAPK3; SRC; RAF1; |
| Signaling | MAP2K2; STAT3; MAP2K1; BRAF; ATF4 |
| Mitochondrial | SOD2; MAPK8; CASP8; MAPK10; MAPK9; CASP9; PARK7; PSEN1; |
| Dysfunction | PARK2; APP; CASP3 |
| Notch Signaling | HES1; JAG1; NUMB; NOTCH4; ADAM17; NOTCH2; PSEN1; NOTCH3; |
| | NOTCH1; DLL4 |
| Endoplasmic Reticulum | HSPA5; MAPK8; XBP1; TRAF2; ATF6; CASP9; ATF4; EIF2AK3; CASP3 |
| Stress Pathway | |
| Pyrimidine Metabolism | NME2; AICDA; RRM2; EIF2AK4; ENTPD1; RRM2B; NT5E; POLD1; NME1 |
| Parkinson's Signaling | UCHL1; MAPK8; MAPK13; MAPK14; CASP9; PARK7; PARK2; CASP3 |
| Cardiac & Beta | GNAS; GNAQ; PPP2R1A; GNB2L1; PPP2CA; PPP1CC; PPP2R5C |
| Adrenergic Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine | PRDX6; PRDX1; TYR |
| and Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cm2; Grk2; Trpa1; Pomc; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; Wnt3a; Wnt4; |
| Neurology | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4fl or Brn3a); Numb; Reln |

The rAAV can have an infectivity to a host cell of at least 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a wildtype AAV or the corresponding wildtype AAV serotype.

The rAAV can comprise a chimeric AAV capsid. A “chimeric” AAV capsid refers to a capsid that has an exogenous amino acid or amino acid sequence. The rAAV can comprise a mosaic AAV capsid. A “mosaic” AAV capsid refers to a capsid that made up of two or more capsid proteins or polypeptides, each derived from a different AAV serotype. The rAAV can be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype is pseudotyped, which means that the ITRs from a first AAV serotype (e.g., AAV2) are used in a capsid from a second AAV serotype (e.g., AAV9), wherein the first and second AAV serotypes are not the same. As a non-limiting example, a pseudotyped AAV serotype comprising the AAV2 ITRs and AAV9 capsid protein can be indicated AAV2/9. The rAAV can additionally, or alternatively, comprise a capsid that has been engineered to express an exogenous ligand binding moiety (e.g., receptor), or a native receptor that is modified.

### Methods of Treatment, Formulations, Dosages, and Routes of Administration

Disclosed herein include compositions. In some embodiments, the composition comprises a variant AAV capsid protein provided herein, an AAV capsid provided herein, and/or an rAAV provided herein; and a pharmaceutically acceptable carrier. The pharmaceutical composition can be for intraventricular, intraperitoneal, intraocular, intravenous, intraarterial, intranasal, intrathecal, intracistemae magna, or subcutaneous injection, and/or for direct injection to any tissue in the body. The pharmaceutical composition provided herein can comprise a therapeutic agent. The pharmaceutical composition provided herein can comprise (i) a targeting molecule or a nucleic acid encoding the targeting molecule and/or (ii) a donor nucleic acid or a nucleic acid encoding the donor nucleic acid. The targeting molecule can associate with the programmable nuclease. The targeting molecule can comprise single strand DNA or single strand RNA. The targeting molecule can comprise a single guide RNA (sgRNA).

Also disclosed herein are pharmaceutical compositions comprising one or more of the compositions provided herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). The physiologically acceptable carrier is an aqueous pH buffered solution.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. The beneficial response can be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration can be changed, or an additional agent can be administered to the subject, along with the therapeutic rAAV composition. As a patient is started on a regimen of a therapeutic rAAV composition, the patient is also weaned off (e.g., step-wise decrease in dose) a second treatment regimen.

Pharmaceutical compositions in accordance with the present disclosure can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations can be converted to vg or viral genomes per kg or into total viral genomes administered by one of skill in the art.

A dose of the pharmaceutical composition can comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10₁₀, 2×10¹¹, 2×10¹², 2×10¹³, 2×10¹⁴, 2×10¹⁵, 2×10¹⁶, 2×10¹⁷, or a range between any two of these values. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, 4×10¹⁷, or a range between any two of these values. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10⁶, 7×10¹⁷, or a range between any two of these values. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

Disclosed herein includes formulations of pharmaceutically-acceptable excipients and carrier solutions suitable for delivery of the rAAV compositions described herein, as well as suitable dosing and treatment regimens for using the particular compositions described herein in a variety of treatment regimens. The amount of therapeutic gene expression product in each therapeutically-useful composition can be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens can be desirable. In some instances, the rAAV compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions can be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Pulmonary administration can be advantageously achieved via the buccal administration. Formulations can comprise dry particles comprising active ingredients. Dry particles can have a diameter in the range from about 0.5 nm to about 7 nm or from about 1 nm to about 6 nm. Formulations can be in the form of dry powders for administration using devices comprising dry powder reservoirs to which streams of propellant can be directed to disperse such powder. Self-propelling solvent/powder dispensing containers can be used. Active ingredients can be dissolved and/or suspended in low-boiling propellant in sealed containers. Such powders can comprise particles, in which at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions can include a solid fine powder diluent such as sugar and are conveniently provided in a unit dose form. Low boiling propellants generally include liquid propellants having a boiling point of below 65° F. at atmospheric pressure. Generally, propellants can constitute 50% to 99.9% (w/w) of the composition, and active ingredient can constitute 0.1% to 20% (w/w) of the composition. Propellants can further comprise additional ingredients such as liquid non-ionic and/or solid anionic surfactant and/or solid diluent (which can have particle sizes of the same order as particles comprising active ingredients).

Pharmaceutical compositions formulated for pulmonary delivery can provide active ingredients in the form of droplets of solution and/or suspension. Such formulations can be prepared, packaged, and/or sold as aqueous and/or dilute alcoholic solutions and/or suspensions, optionally sterile, comprising active ingredients, and can conveniently be administered using any nebulization and/or atomization device. Such formulations can further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration can have an average diameter in the range from about 0.1 nm to about 200 nm. Formulations described herein useful for pulmonary delivery can also be useful for intranasal delivery. Formulations for intranasal administration comprise a coarse powder comprising the active ingredient and having an average particle size from about 0.2 μm to 500 μm. Such formulations are administered in the manner in which snuff is taken, e.g. by rapid inhalation through the nasal passage from a container of the powder held close to the nose.

Formulations suitable for nasal administration may, for example, comprise from about as little as 0.1% (w/w) and as much as 100% (w/w) of active ingredient, and can comprise one or more of the additional ingredients described herein. A pharmaceutical composition can be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration can comprise powders and/or an aerosolized and/or atomized solutions and/or suspensions comprising active ingredients. Such powdered, aerosolized, and/or aerosolized formulations, when dispersed, can comprise average particle and/or droplet sizes in the range of from about 0.1 nm to about 200 nm, and can further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions will be within the purview of the skilled artisan having benefit of the present teachings. It is likely, however, that the administration of therapeutically-effective amounts of the disclosed compositions can be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In some embodiments, it can be desirable to provide multiple, or successive administrations of the AAV vector compositions, either over a relatively short, or a relatively prolonged period of time, as can be determined by the medical practitioner overseeing the administration of such compositions. For example, the number of infectious particles administered to a mammal can be on the order of about 10⁷, 10⁸, 10⁹, 10¹⁰, 10¹¹, 10¹², 10¹³, or even higher, infectious particles/ml given either as a single dose, or divided into two or more administrations as can be required to achieve therapy of the particular disease or disorder being treated. In fact, it can be desirable to administer two or more different AAV vector compositions, either alone, or in combination with one or more other therapeutic drugs to achieve the desired effects of a particular therapy regimen. The daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The administration of the therapeutic rAAV composition can be hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, 5 years, or 10 years. The effective dosage ranges can be adjusted based on subject's response to the treatment. Some routes of administration will require higher concentrations of effective amount of therapeutics than other routes.

Although not anticipated given the advantages of the present disclosure, in some cases that the patient's condition does not improve, upon the doctor's discretion the administration of therapeutic rAAV composition is administered chronically, that is, for an extended period of time, including throughout the duration of the patient's life in order to ameliorate or otherwise control or limit the symptoms of the patient's disease or condition. A patient's status does improve, the dose of therapeutic rAAV composition being administered can be temporarily reduced or temporarily suspended for a certain length of time (i.e., a “drug holiday”). The length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. The dose of drug being administered can be temporarily reduced or temporarily suspended for a certain length of time (i.e., a “drug diversion”). The length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. However, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. The data obtained from cell culture assays and animal studies are used in formulating the therapeutically effective daily dosage range and/or the therapeutically effective unit dosage amount for use in mammals, including humans. The dosage amount of the therapeutic rAAV composition described herein lies within a range of circulating concentrations that include the ED50 with minimal toxicity. The daily dosage range and/or the unit dosage amount varies within this range depending upon the dosage form employed and the route of administration utilized.

Disclosed herein include methods of introducing a nucleic acid into a cell. In some embodiments, the method comprises: contacting the cell with a variant AAV capsid provided herein, or the therapeutically effective amount of the rAAV provided herein, or the composition provided herein. The cell can be present in a subject. Introducing the nucleic acid into the cell can comprise: (i) isolating one or more cells from the subject; (ii) contacting said one or more cells with a composition comprising; and (iii) administering the one or more cells into a subject after the contacting step. The contacting can be performed in vivo, in vitro, and/or ex vivo. The contacting can comprise calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. The subject can be a mammal. The mammal can be a human. Contacting one or more cells with the composition comprising a variant AAV capsid provided herein, or a therapeutically effective amount of a rAAV provided herein, or a pharmaceutical composition provided herein, is in a subject's body. The one or more cells are contacted with the composition comprising a variant AAV capsid provided herein, or a therapeutically effective amount of a rAAV provided herein, or a pharmaceutical composition provided herein, in a cell culture.

Disclosed herein include methods of treating a disease or disorder in a subject. In some embodiments, the method comprises: administering to the subject a therapeutically effective amount of an rAAV provided herein. The administering can comprise systemic administration. The systemic administration can be intravenous, intramuscular, intraperitoneal, or intraarticular. Administering can comprise intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The method provided herein can comprise administering an inducer of the inducible promoter to the one or more cells. The inducer can comprise doxycycline.

Administering can comprise an injection into a brain region. Administering can comprise direct administration to the brain parenchyma. For example, the brain region can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof.

The brain region can comprise neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof. Administering can comprise delivery to dorsal root ganglia, visceral organs, astrocytes, neurons, or a combination thereof of the subject.

The variant AAV capsid can have tropism for a target tissue or a target cell. The target tissue or the target cell can comprise a tissue or a cell of a CNS or a PNS, or a combination thereof. The target cell can be a neuronal cell, a neural stem cell, an astrocyte, a tumor cell, a hematopoetic stem cell, an insulin producing beta cell, a lung epithelium, a skeletal cell, or a cardiac muscle cell. The target cell can be located in a brain or spinal cord. The target cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can be an embryonic stem cell, an induced pluripotent stem cell (iPSC), a hematopoietic stem/progenitor cell (HSPC), or a combination thereof.

The disease or disorder can be pulmonary fibrosis, surfactant protein disorders, peroxisome biogenesis disorders, or chronic obstructive pulmonary disease (COPD). The disease or disorder can comprise a central nervous system (CNS) disorder or peripheral nervous system (PNS) disorder. The subject can be a subject suffering from or at a risk to develop one or more of chronic pain, cardiac failure, cardiac arrhythmias, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, and lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

The disease or disorder can comprise a neurological disease or disorder. For example, the neurological disease or disorder can be epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof. The disease or disorder can be any one of the diseases or disorders shown in the tables provided herein. Diseases and disorders contemplated herein include cystic fibrosis, glycogen storage disease III, nonsyndromic deafness, dysferlinopathies, Usher 1b, Stargardt disease, Hemophilia A, Lebar congenital amaurosis, Usher 1d, Duchenne Muscular Dystrophy, and Alstrom Syndrome.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1-2 described below.

Construction of Plasmids

The InFusion (Takara) assembly method was used for the construction of plasmids used in this study. The plasmid backbones were digested with commercial restriction enzymes (New England Biolabs), the inserts were synthesized (Integrated DNA Technology and Wuxi Qinglan Biotech) and/or amplified with Q5 high-fidelity DNA polymerase (New England Biolabs), and the primers were synthesized by Integrated DNA Technologies (Integrated DNA Technology). The digested backbone and inserts were purified with agarose gel purification. InFusion (Takara) assembly products were transformed into either Stb13 (Invitrogen) or NEB Stable (New England Biolabs) competent cells. Sanger sequencing was used to confirm the correct insert sequences (performed by Laragen).

Cell Culture and Production of Viral Particle Extracts

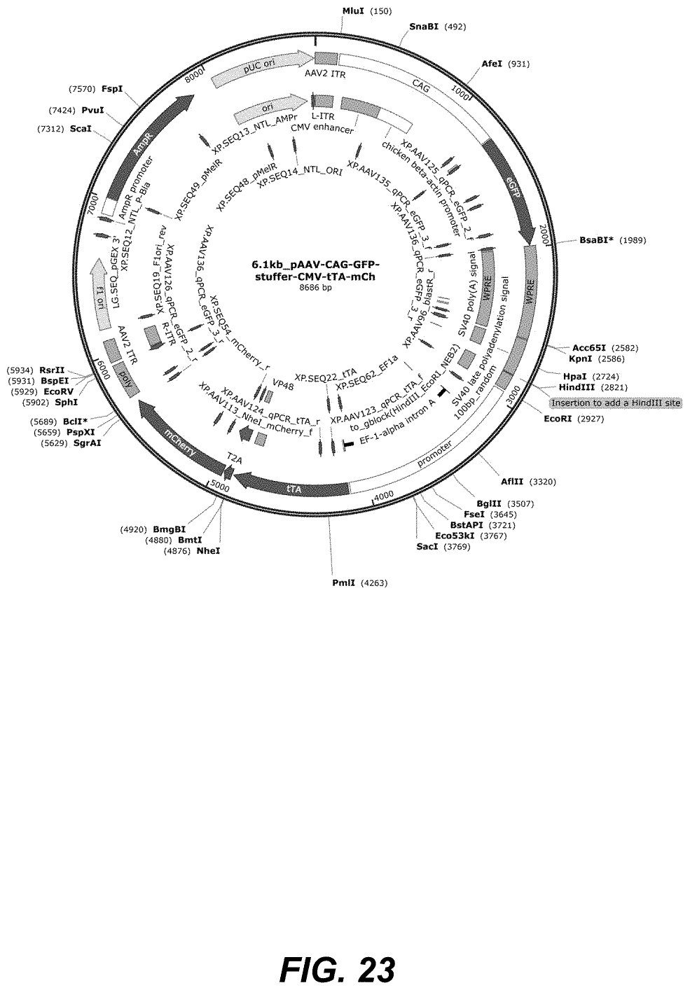

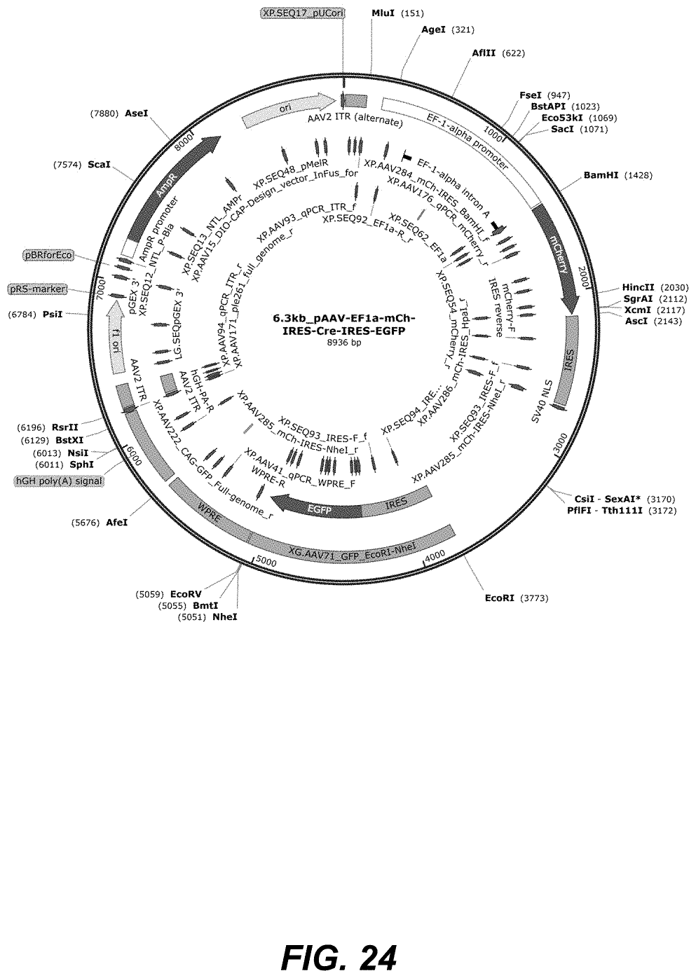

HEK293T cell culture and triple transfection with polyethylenimine (PEI) were conducted according to a published protocol. HEK293T cells were cultured in DMEM (ThermoFisher, cat. no. 10569044) with 5% FBS, 10 mM HEPES, and 1× non-essential amino acids. The cells were passaged to 15-cm dishes or 6-well plates at 40% confluence one day before transfection. In the case of 15-cm dishes, HEK293T producer cells (˜80-90% confluent) were transfected with PEI with 20 μg iCAP or REP-CAP plasmid, 10 μg pHelper plasmid (Agilent, cat. no. 240071), 5 μg CMV-AAP plasmid, and 5 μg rAAV genome plasmid. In the case of 6-well plates, the plasmid masses were linearly scaled down to 2 μg iCAP plasmid, 1 μg pHelper plasmid, 0.5 μg CMV-AAP plasmid, and 0.5 μg rAAV genome plasmid for each well. Plasmid maps for rAAV genomes (SEQ ID NO: 110 and SEQ ID NO: 111) are shown in FIG. 23 and FIG. 24. Plasmids were transfected to cells with PEI at a 3.5:1 (μg PEI: μg DNA) ratio.

72-96 hr after transfection, 1/80 media volume of 0.5 M EDTA was added to each dish/well to detach the cells from culture dishes. After 15 min incubation at room temperature, the mixture of cells and media was collected and centrifuged at 2000 g for 10 min at 4° C., and the supernatant (media) was removed completely.

Producer cell pellets were immediately lysed following a generic protocol for nonenveloped viruses with some modifications. Lysis buffer (100 mM magnesium chloride, 38.1 mM citric acid, 74.8 mM sodium citrate, 75 mM sodium chloride; the pH of the resulting solution should be ˜4) was sterilized with a 0.22 μm PES, supplemented with 1 tablet protease inhibitor (ThermoFisher, cat. no. A32963) per 50 mL solution, and stored at 4° C.

The cell pellet samples were lysed with two rounds of lysis cycles. In each round, fresh lysis buffer was added to cell pellets at a ratio of 1 mL/1e7 cells. The mixture was vortexed for 20 s to fully resuspend the pellet. The resulting sample was then incubated for 5-15 min at room temperature and vortexed for 20 s again before being centrifuged at 5000-9000 g for 10 min. The supernatant was moved to a clean tube. Supernatant from two rounds of lysis were combined, and the total volume of the extract solution should be ˜2 mL/1e7 cells. The extract solution was neutralized with 1/10 volume of neutralization buffer (2 M Tris-HCl, pH 9.5) and then diluted with 1/3 volume of ddH₂O. Depending on the purpose of the experiment, the samples were either further purified or used for experiments, as specified in figure legends. In cases when the sample was not being used for further purification, 0.05% BSA was added to the sample for better stability.

Virus Purification (Precipitation-Based Method)

Unless otherwise specified, virus samples for TEM, DLS, and transduction assays were prepared via the following precipitation-based purification method, a modified version of a generic protocol for purifying nonenveloped viruses. The viral particle extract was incubated with 1/100 volume of DNase I (Roche, cat. no. 4716728001) and 1/100 volume of MspI (NEB, cat. no. R0106L). The reaction was incubated at 37° C. for 1 hr. Afterward, the sample was mixed well with 1/10 volume of precipitation buffer A (5% (m/v) sodium deoxycholate), and the mixture was incubated at 37° C. for another 30 min. Finally, the sample was mixed with 1/25 volume of precipitation buffer B (1 M citric acid), vortexed for 10 s, and centrifuged at 5000 g for 5 min at 4° C. All above-mentioned “volumes” refer to the total liquid volume at the step.

After centrifugation, the supernatant with capsids was filtered with a 0.45 μm filter (Millipore cat. no. SLHP033RS or cat. no. UFC30HV00) and buffer exchanged for at least 5 cycles with 100 kD MWCO centrifugal concentrator (Thermo Scientific, cat. no. 88503 or cat. no. 88533) to the final storage buffer (PBS with 300 mM additional NaCl and 0.001% Pluronic F-68 non-ionic surfactant (Thermo Scientific, cat. no. 24040032)). In each cycle of buffer exchange, the concentrator tubes were centrifuged at 1000 g, 4° C. for 20 min or more to reduce the solution volume by at least 5 folds. The purified virus samples were titrated with qPCR at the same day, and the remaining samples were stored at 4° C. for further characterization.

For TEM and DLS analyses, sample purity was further improved by 3-5 cycles of buffer exchange with a 300 kD MWCO centrifugal concentrator (Pall, Cat. OD300C33). Note that, in some embodiments, this step's recovery rate is suboptimal (<5% depending on the variant being purified).

Virus Purification (Affinity-Based Method)

The viral particle extract was incubated with 1/100 volume of DNase I (Roche, cat. 4716728001) and 1/100 volume of MspI (NEB, cat. no. R0106L). The reaction was incubated at 37° C. for 1 hr and then incubated with POROS CaptureSelect AAV9 Affinity Resin (ThermoFisher Scientific, cat. A27356) at a ratio of 200 μL slurry per 1 mL extract at room temperature for 2 hr on a rotator. The resulting resins were collected by centrifugation at 500 g for 5 min. The supernatant was discarded, and the resin pellets were washed with 10× slurry volume of PBS and centrifuged again at 500 g for 5 mins. The resulting resin pellets were loaded into a micro-spin column (˜100 μL slurry in each column) and washed with 300 μL wash buffer 1 (PBS+additional 100 mM NaCl) twice and 300 μL wash buffer 2 (PBS+additional 250 mM NaCl+0.025% Tween 20) once. The bound virus particles were then incubated with 100 μL of elution buffer (2 M MgCl2) at room temperature for 15 min before centrifuged at 500 g for 5 min. The eluted solution was collected and used for TEM imaging.

qPCR Titration of Viral Genome Copy Number Protected from DNase I Digestion

qPCR titration was performed in triplicate for each sample. As an overview, the samples were first treated with DNaseI to remove unpackaged genome DNA, and then the capsid-protected genomes were released by proteinase K digestion and heat denaturation. DNaseI solution was prepared fresh by diluting DNaseI recombinant (RNase-free; 10 U/μL; Roche Diagnostics, cat. no. 4716728001) 200-fold in 1× DNaseI buffer supplied by the manufacturer to a final concentration of 50 U/mL. Proteinase K solution was prepared fresh by dilution of Proteinase K (recombinant, PCR grade; 50 U/mL (2.5 U/mg); Roche Diagnostics, cat. no. 03115828001) 200-fold in Proteinase K buffer (1 M NaCl and 1% (wt/vol) N-lauroylsarcosine sodium salt) to a final concentration of 0.25 U/mL. 2 μL of each sample replicate was treated with 50 μL DNaseI solution for 30 min at 37° C. before inactivation, which was done by adding 2.5 μL of 5 M EDTA to the sample and heating it at 70° C. for 10 min. Each reaction was then added to 60 μL of Proteinase K solution and incubated at 56° C. for 2 hr or overnight. The resulting reaction was inactivated at 95° C. for 10 min. Depending on the starting material, the treated sample was either diluted 300-fold in water or column purified (eluted in 10 μL TE buffer) before qPCR titration so that the resulting CT values can fit in the linear dynamic range of the qPCR assay. The qPCR reaction was composed of 12.5 μL of SYBR Green master mix (Roche Diagnostics, cat. no. 04913850001), 9.5 μL of water, 0.5 μL of each primer (from a 2.5-04 stock concentration), and 2 μL of the diluted or purified sample. A ˜100 bp amplicon in the rAAV genome was amplified for quantification. The thermocycler parameters were: (1) 95° C. for 10 min, (2) 95° C. for 15 s, (3) 60° C. for 20 s, (4) 60° C. for 40 s, (5) Repeat steps 2-4 for 40 cycles.

Rosetta Modeling of HI-Loop-Shortened Variants

12 AAV9 variants with shortened HI loops were designed by removing a step series of peptides from the center of the loop and refilling the gap with different numbers of flexible residues (glycine/serine). The new designs were then evaluated with RosettaRemodel with the cyclic coordinate descent (CCD) closure algorithm. In brief, a trimer model of AAV9 capsid protein (PDB ID: 3ux1) was used as the modeling template. In the modeling blueprint, 4 residues adjacent to the truncation site (2 residues on each side) and the flexible (glycine/serine) peptide were set to be movable, while the rest of the capsid protein trimer was fixed. the Rosetta design score reported for the 5 lowest-energy poses as well as the total number of accepted poses for each design calculated within 3 hours with the same computational hardware settings were recorded. The relative design scores were calculated as the absolute value of the difference between each variant and the variant with the highest score.

Negative Staining Transmission Electron Microscopy (TEM) and Quantification

Ultrathin carbon EM grids (Ted Pella, cat. no. 01822-F) were glow discharged and positioned with a pair of reverse tweezers. A 3-μL droplet of capsid samples was placed onto the grids for 30 s-1 min and blotted dry with filter paper. A 3-μL droplet of 2%-3% uranyl acetate solution was then placed onto the grid to stain the sample for 30 s before being blotted dry with filter paper. The grids were imaged with an FEI Tecnai T12 120 kV electron microscope equipped with a Gatan 2k×2k CCD.

The size and shape of particles in the TEM images were segmented and quantified with a customized Python script. Briefly, the TEM images were normalized, and a mask that labels pixels corresponding to individual particles was generated using the Cellpose algorithm. Segmentation artifacts and outliers were removed by filtering out any particle with perimeter, roundness, or area that were two standard deviations away from the mean. Segmentation results were manually inspected to make sure that real particles were correctly identified. The size and shape parameters of the particles were then measured and calculated on the basis of the mask.

In Vitro Transduction Assay in Mammalian Cells

For transduction assay with HEK293T cells, the cells were passaged to a T75 flask at 40% confluence in DMEM media with 5% FBS. 24 hr later, the cells transfected with 7 μg pHelper plasmid (transfection marker) with PEI. 4-6 hr later, the cells were seeded to 96-well plates at ˜10,000 cells/well in 80 μL DMEM media DMEM media with 5% FBS. 2 hr after seeding, each well of cells was treated with 60 μL of viral samples carrying a 6.1 kb rAAV genome (pAAV-CAG-GFP-spacer-CMV-mCherry). The plasmid map for pAAV-CAG-GFP-spacer-CMV-mCherry is shown in FIG. 23. Unless otherwise specified in the text, viral samples in the media of the virus producer cells were DNase-treated. In detail, culture media of producer cells was treated with 0.1 U/uL DNaseI for 1-2 hr, thermal-ablated at 45° C. for 1 hr to remove residual live cells and centrifuged at 9000 g for 10 min to remove aggregations before being added to the cells. After 18 hr of incubation, the media was replaced by 200 μL of imaging media (Fluorobrite DMEM (ThermoFisher, cat. no. A1896701) with 1% FBS, 4 mM GlutaMAX, 10 mM HEPES). Media was replaced every 2-3 days. Images were taken 4 days after transduction with a LSM800 inverted confocal microscope.

### Example 1

### Rational Design Based on Structural Dissection Yields AAV Capsids with Expanded Sizes

Structural Dissection of an AAV Capsid Subunit Provides Insights on Sites that can be Engineered to the Alter Capsid Assembly Pathway

Without being bound by any particular theory, because of the apparently monolithic structure of an AAV capsid subunit, the protein has not been dissected into modular, independently folding domains. This makes it difficult to tune each different type of inter-subunit interaction (i.e. 2-fold, 3-fold, and 5-fold symmetrical interactions) independently, posing a challenge to the engineering of the self-assembly process of the capsid protein. Without being bound by any particular theory, provided herein is the first dissection of the full-length capsid protein into structural blocks based on their roles in capsid assembly.

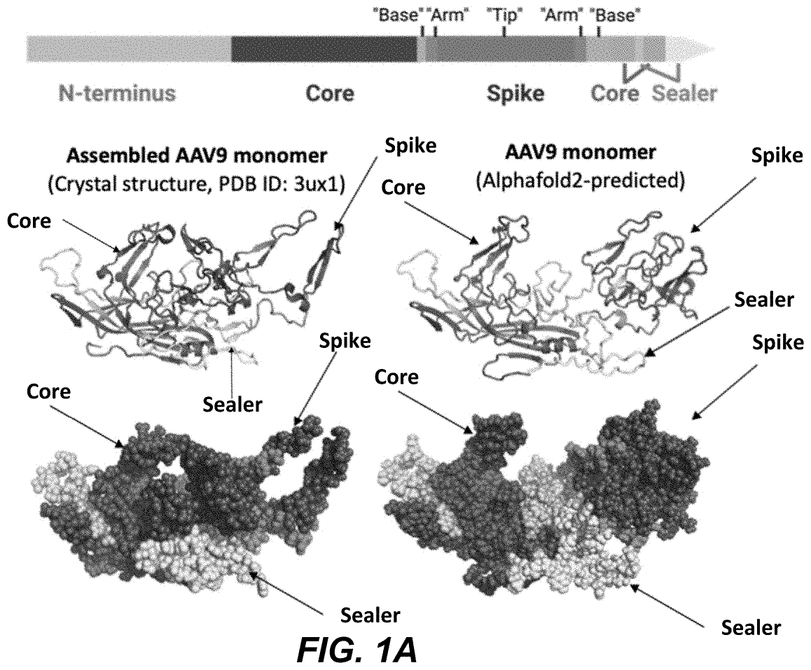

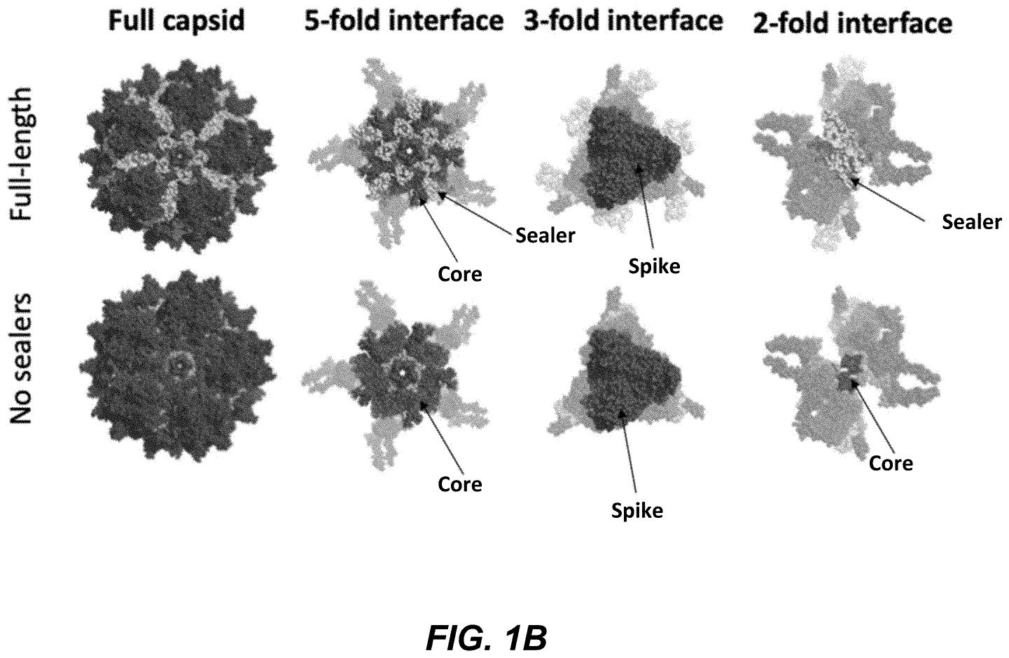

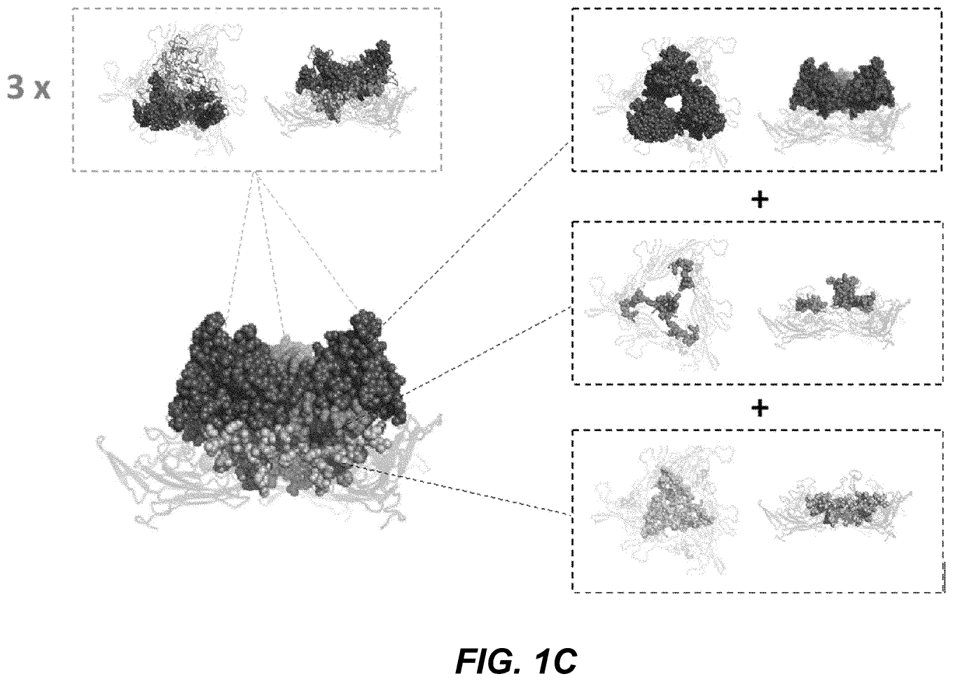

With careful analysis of residues involved in each different symmetrical interaction in an assembled AAV capsid structure, each AAV capsid subunit was split into four major “blocks” by their assembly-related roles (FIG. 1A-FIG. 1B, Table 4). For simplicity of discussion, these four blocks are referred to as disordered N-terminus block (residues 1-218), core block (residues 219-417, 641-691), spike block (residues 418-640), and sealer block (residues 692-736) (Table 4). For simplicity, the residue indices discussed in the present disclosure all refer to the indices in AAV9 VP1 coding sequence. In some embodiments, the corresponding residues in other AAV serotypes can shift slightly according to sequence alignments. Except for the disordered N-terminal block, each of the other blocks is involved in one or two types of symmetrical interactions that are essential for assembling into an icosahedral capsid. The core block is further divided into the primary segment and two auxiliary segments because of their separation in the primary amino acid sequence. The spike block is further divided into three types of segments, “base”, “arm”, and “tip”, based on the different type of interactions they are involved in (FIG. 1C, Table 4). The “base”, “arm”, and “tip” segments together form a 3-layered structure. A few internal “hinges” within the spike block that connects blocks, segments, or sub-segments at the boundaries, many with G/S/P-rich flexible peptide sequences (Table 5), were identified. In some embodiments, these “hinge” sites can be more tolerable to engineering.

TABLE 4
STRUCTURAL DISSECTION OF AN AAV CAPSID SUBUNIT

| | | | Main roles in capsid assembly-related interactions | |
|---|---|---|---|---|
| Name | | Approximate | | Involved |
| Block | Segment | range of amino | | type(s) of |
| name | name | acid indices | Description | interaction |
| N- | | 1-218 (218aa) | Disordered residues unlikely contributing | N.A. |
| terminal | | | to capsid assembly | |
| block | | | | |
| Core | Primary | 219-417 (199aa) | Forms the main part of the core jelly-roll | Jelly roll |
| block | segment | | fold (beta strands B through G) | formation, |
| | Auxiliary | 641-655 (15aa) | Supports the core jelly-roll fold with beta | 5-fold |
| | segments | 670-691(22aa) | strands H and I | interactions, 2- |
| | | | | fold |
| | | | | interactions |
| Spike | Spike | 418-428 (11aa) | Binds to counterpart residues in two 3-fold | 3-fold |
| block | “base” | 611-640 (30aa) | neighbors | interactions |
| | segments | | | |
| | Spike | 429-443 (15aa) | Forms simple 3-fold interactions | |
| | “arm” | 593-610 (18aa) | | |
| | segments | | | |
| | Spike “tip” | 444-592 (149aa) | Forms intertwined 3-fold interactions with | |
| | segment | | counterpart residues in two 3-fold | |
| | | | neighbors | |
| Sealer | “5-fold | 656-669 (14aa) | Forms a “caulk” that seals the capsid | 5-fold |
| block | sealer” | | surface near 5-fold axis. | interaction |
| | “2-fold | 692-736 (45aa) | Forms a “caulk” that seals the capsid | 2-fold |
| | sealer” | | surface near 2-fold axis. The segment | interactions, |
| | | | interacts with counterpart residues in a 2- | 3-fold |
| | | | fold neighbor and forms weak interaction | interactions, 5- |
| | | | with the core blocks of a 5-fold neighbor | fold |
| | | | and a 3-fold neighbor. | interactions |

TABLE 5
INTERNAL “HINGES” WITHIN THE SPIKE BLOCK

| Sub-segment | Approximate range of | |
|---|---|---|
| name | amino acid indices | Main roles in capsid assembly-related interactions |
| Internal | 424-428 (5aa) | Connects blocks, segments, or sub-segments at the |
| “Hinges” | 431-433 (3aa) | boundaries. The hinges are typically rich of G, S, and P |
| | 444-445 (2aa) | residues, providing, in some embodiments, flexibility to |
| | 466-468 (3aa) | make turns. |
| | 480-482 (4aa) | |
| | 504-506 (3aa) | |
| | 521-523 (3aa) | |
| | 571-578 (8aa) | |
| | 593-594 (2aa) | |
| | 603-610 (8aa) | |
| | 636-640 (5aa) | |

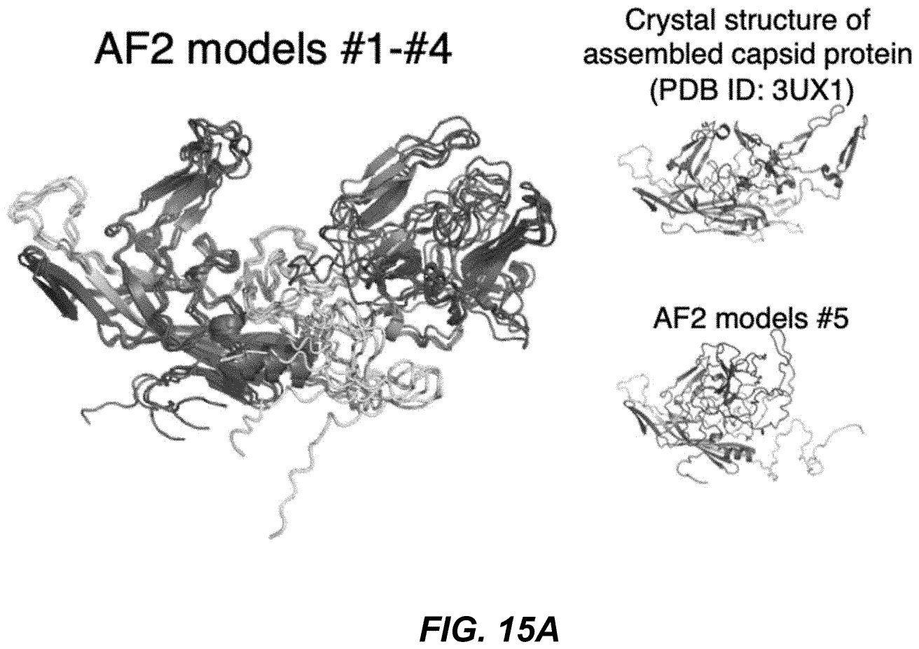

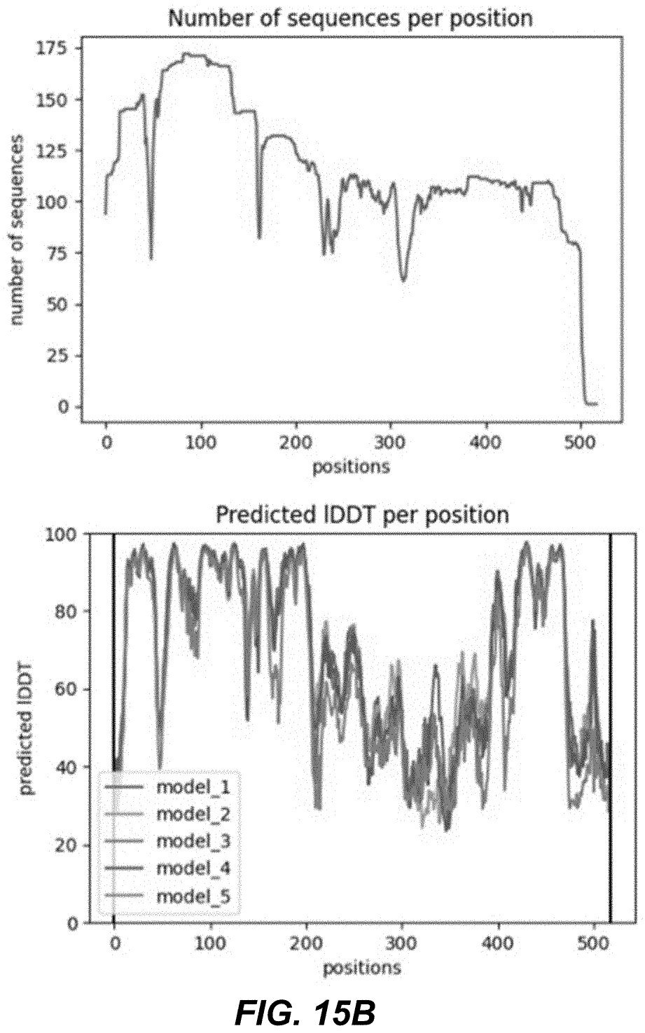

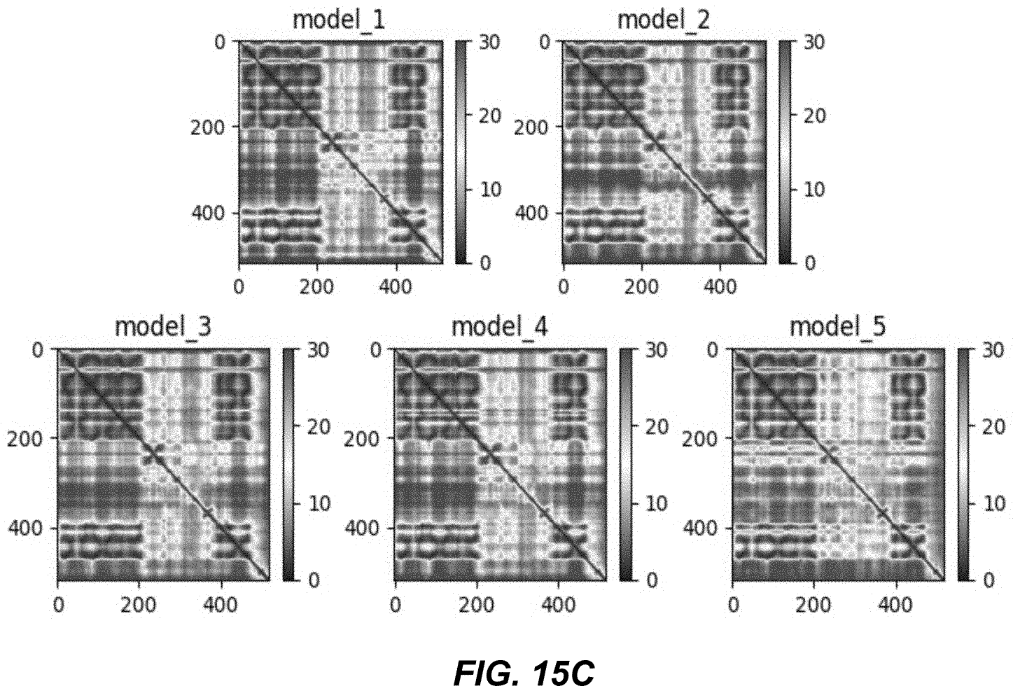

The structure of an AAV9 capsid subunit was also analyzed using an automated pipeline for multiple-sequence alignment (MSA) and structure prediction based on a trained Alphafold 2 (AF2) model, which has been shown to be highly accurate in predicting structures of soluble proteins. Only the MSA, but no template pdb structure, was provided as the input feature for the AF2 model to avoid bias introduced by available AAV crystal structures. Interestingly, the top 4 of the 5 predicted structures (ranked by average local Distance Difference Test, 1DDT, score) converged well into a structure that looked different from the crystal structure of AAV9 capsid protein in an assembled capsid (FIG. 1A, FIG. 15A-FIG. 15C). Given the convergence and the overall high confidence score of the AF2 models, without being bound by any particular theory, these models can, in some embodiments, provide insights into the pre-assembly monomeric state of AAV capsid proteins.

Compared to the crystal structure (PDB ID: 3UX1), the AF2 model aligns almost perfectly in the core block but shows interesting discrepancies in the spike block and the sealer block (FIG. 1A), which can guide engineering of the capsids. The differences in the sealer block can be explained by lack of binding partners that hold the peptide loops in place. The differences in the spike block appear to be more striking, in some embodiments, AF2 predicted that the “arm” and “tip” segments (approximately residues 429-608) can fold into an independently-folding domain by themselves. This suggests that some common protein engineering strategies applicable to foldable protein domains, such as truncations or domain swaps, can be used to engineer the spike block as well.

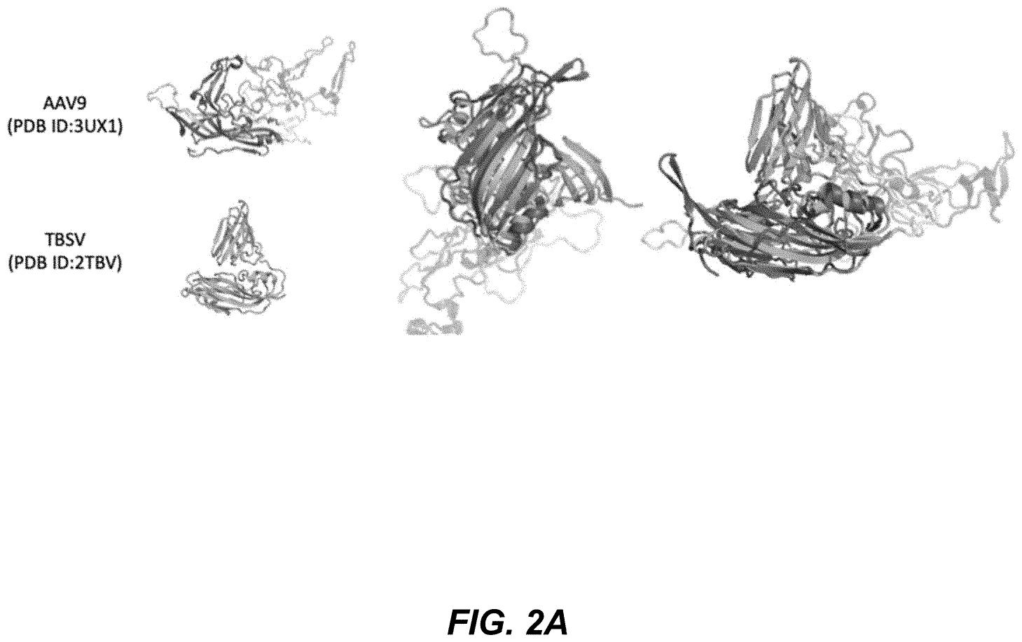

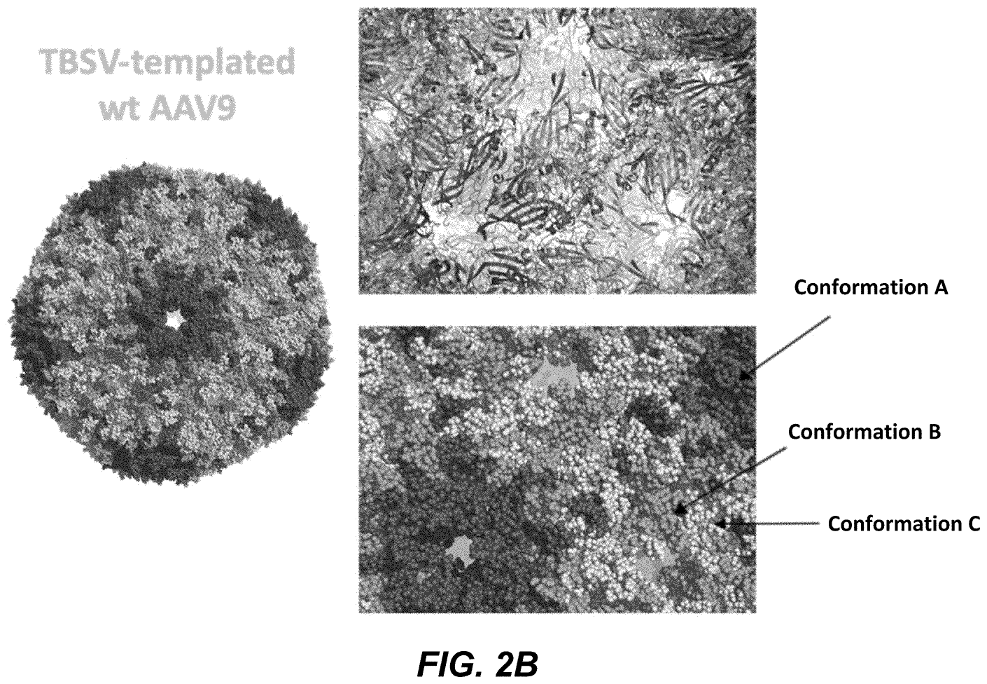

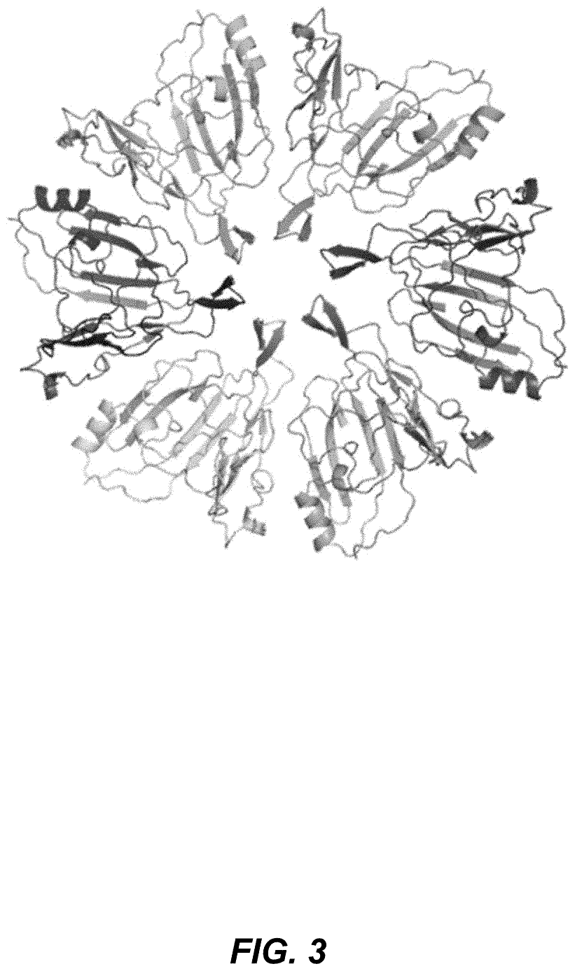

Another interesting comparison was between the AAV capsid structure and that of TBSV, a plant RNA virus with a 32-nm T=3 icosahedral capsid (FIG. 2A-FIG. 2B). The length of the structured part of AAV9 is ˜516 aa (out of a total of 736 aa), while the length of the structured part of TBSV coat protein is ˜287 aa (out of the total 387 aa). The difference in length is mainly contributed by the extra sequences in spike block and the sealer block of an AAV capsid protein. A structural alignment also echoes this, as the shape of the core block of AAV highly resembles the TBSV structure (FIG. 2A-FIG. 2B), and the structural differences between the two proteins also mainly attribute to the spike block and the sealer block. The most notable difference resides in the spike block, especially the “tip” segment of the block, which forms an extraordinarily intertwined 3-fold interface that is absent in a TBSV capsid.

The structural analyses provided information on the role played by each block during the capsid assembly and suggested strategies and sites to engineering the assembly pathways. For example, both comparisons (crystal structure to AF2-predicted structure, AAV9 structure to TBSV structure) suggested that the folding of the spike block (particularly the “arm” and “tip” segments) can fold independently from the core block, and that the sealer block cannot fold until after the capsid is assembled. These structural analyses also suggested that the hyperstable 3-fold interactions formed by the spike block can be the main contributing factors to the different assembly pathways and resulting geometries of the assembly products between the T=1 AAV capsid and other T=3/4 jelly-roll-fold capsids. In some embodiments, self-assembly products of AAV capsid proteins can be altered by removal or modification of these interactions specifically.

C-Terminal-Truncated Capsid Subunit Design Concept

This example provides validation that the core block, particularly the primary segment, contains the minimal sequence needed for forming genome-protecting assemblies.

The structural dissection showed that the N-terminal half of the AAV subunit (residues 1-417) covers most of the core block that is needed to form the jelly-roll structure as well as 5-fold interactions. Without being bound by any particular theory, a C-terminal-truncated capsid subunit that retains the primary segment of the core block can self-assemble and protect the rAAV genome.

With Alpha-fold2, the structure of AAV9 Δ426-736, a truncation that kept only the primary segment of the core block (FIG. 1A-FIG. 1C, Table 4) was predicted. Indeed, the program predicted that the truncated capsid protein can fold in a similar way to that of a wtAAV capsid protein or a TBSV coat protein. Interestingly, Alphafold2 predicted that 6 copies of the truncated protein would form a hexamer with the natively pentameric interface. This kind of capsomere-like complex can provide closed structures that protect rAAV genomes.

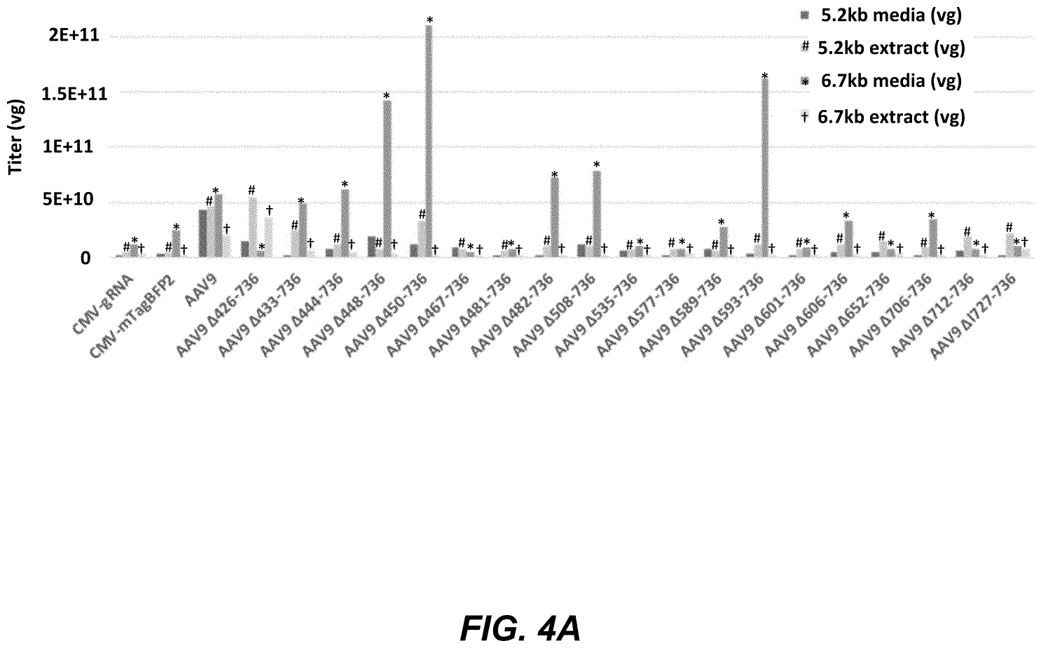

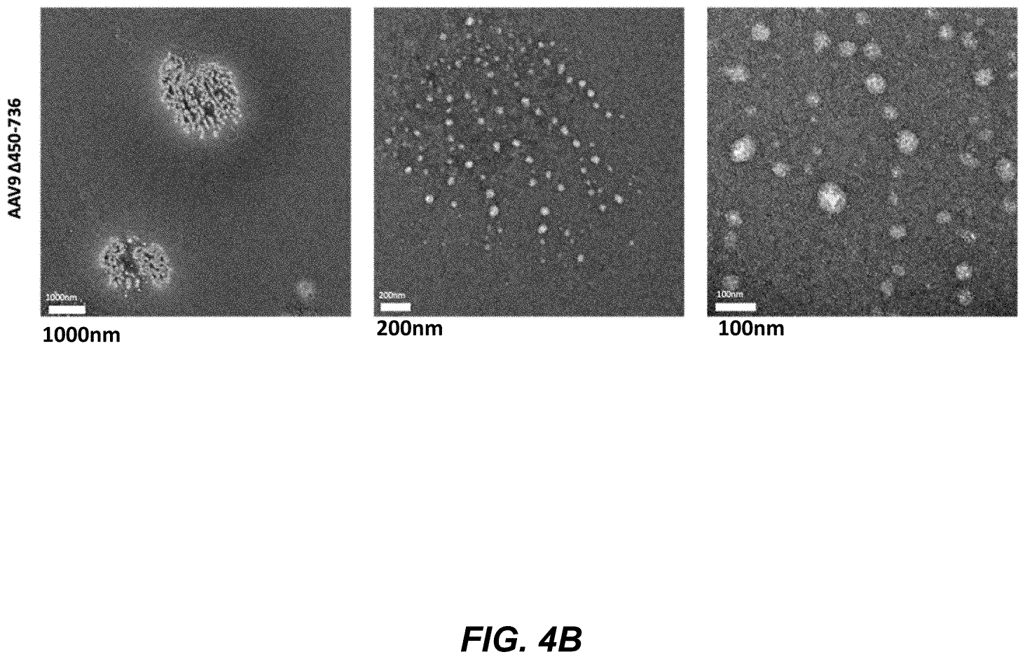

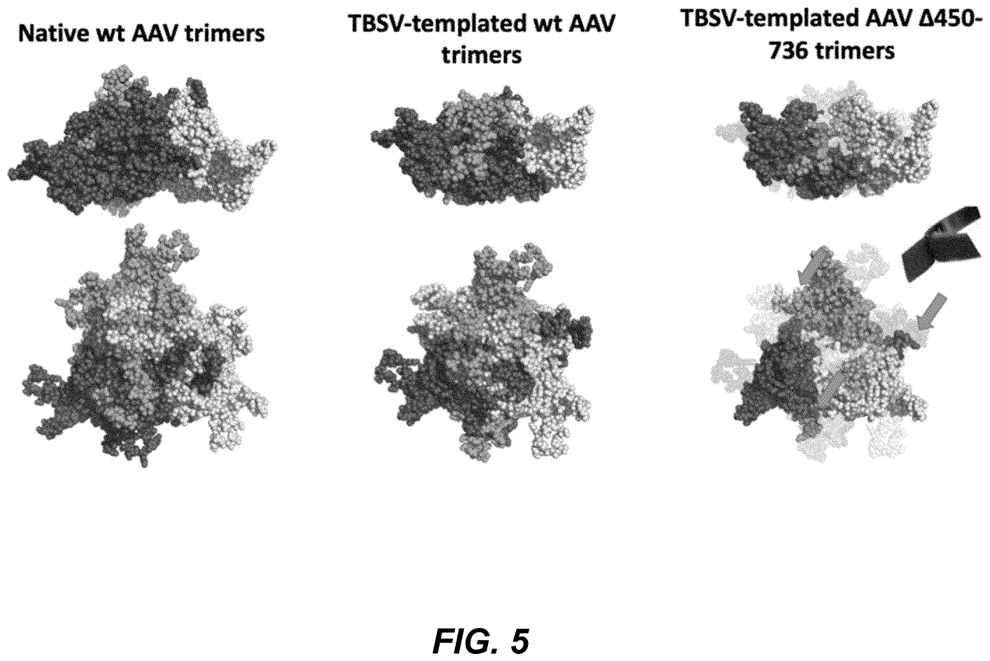

Capsids with a series of capsid proteins with C-terminal truncations were produced with the standard triple-transfection method. The media and cellular extracts from the producer cells were then treated with DNaseI and titered with qPCR to determine whether the capsid proteins can protect the genomes. Interestingly, several C-terminus-truncated capsids could produce DNaseI-protected qPCR titers with both a fully-loading (5.2 kb) genome and an oversized (6.7 kb) genome (FIG. 4A). Among them, Variants that were truncated at ˜450th amino acid showed the highest titer when packaging a 6.7 kb genome. The truncation site happens to be close to the “arm” segment and the “tip” segment within the spike block.

The capsids in the cell lysate of the producer cell of AAV9 Δ450-736 capsid were purified using a precipitation-based purification method that was developed for wild-type AAV capsids. Although spherical-capsid-like structures can be found, they tend to form aggregates (FIG. 4B). Without being bound by any particular theory, it may be because that the truncated variants lost too many residues in the C-terminal end that are help stabilizing the individual capsids. In some instances, uncut, overloaded genomes tether these C-terminal truncated capsids because the capsid variants lost the ability to bind to Rep protein, which is necessary to cut out the overloaded genome.

Compared to wtAAV9, the most significant change in a Δ450-736 capsid was the drastically reduced intertwined 3-fold interactions due to the loss of the “tip” segment. Additionally, most residues involved in 2-fold interactions as well as a small portion of residues involved in 5-fold interactions were removed in this variant. Compared to the “core-only” truncation variants like AAV9 Δ426-736 variant, the Δ450-736 variant did preserve one extra “arm” segment (residue 429-444) that binds to its 3-fold partner. This extra 3-fold arm can serve as a “Velcro” that provides otherwise absent 3-fold interactions, which is an essential type of interaction needed for a canonical icosahedral capsid. Without being bound by any particular theory, this explained the drastically increased DNaseI-protected titer in AAV9 capsid formed by Δ450-736 variant versus 4426-736 variant. The flexibility of this 3-fold arm can also increase the degree of freedom during the capsid assembly, explaining the heterogeneity of the assembled capsids.

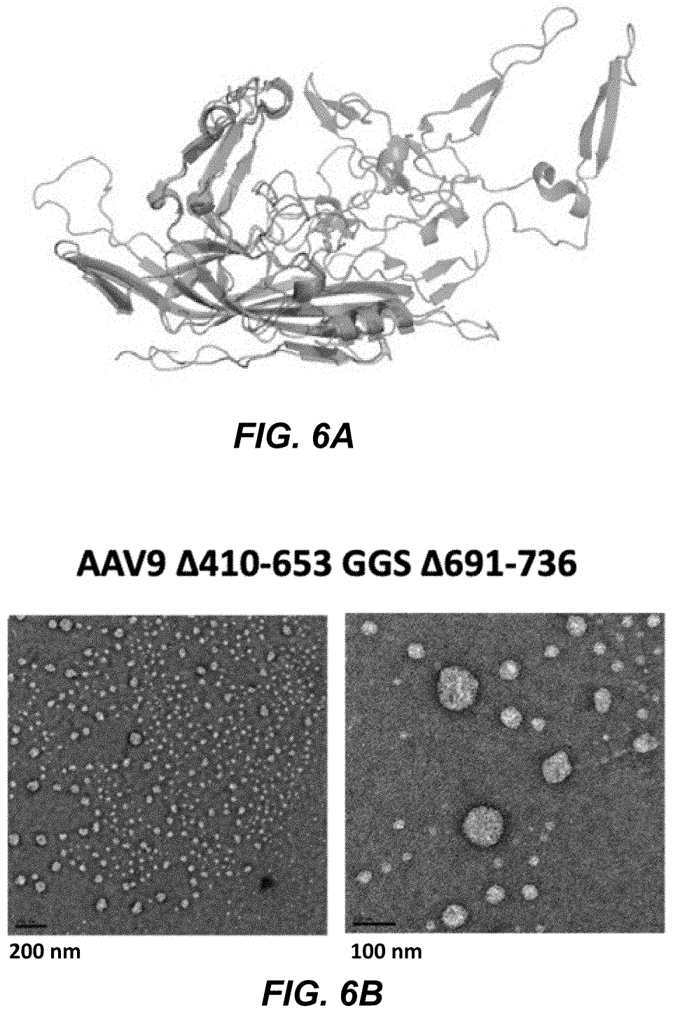

A more rationally truncated core-block-only variant that included both the primary segment and the auxiliary segments was tested. AF2 modeling shows that the design, AAV9 Δ410-653 GGS Δ691-736, indeed variants were rescued with the last two beta strands (strands H and I) that is involved in a canonical jelly-roll protein fold. When expressed, the design showed similar phenotypes as Δ450-736-like variants, as it also made heterogeneous spherical particles, and the particles also tended to aggregate (FIG. 6B).

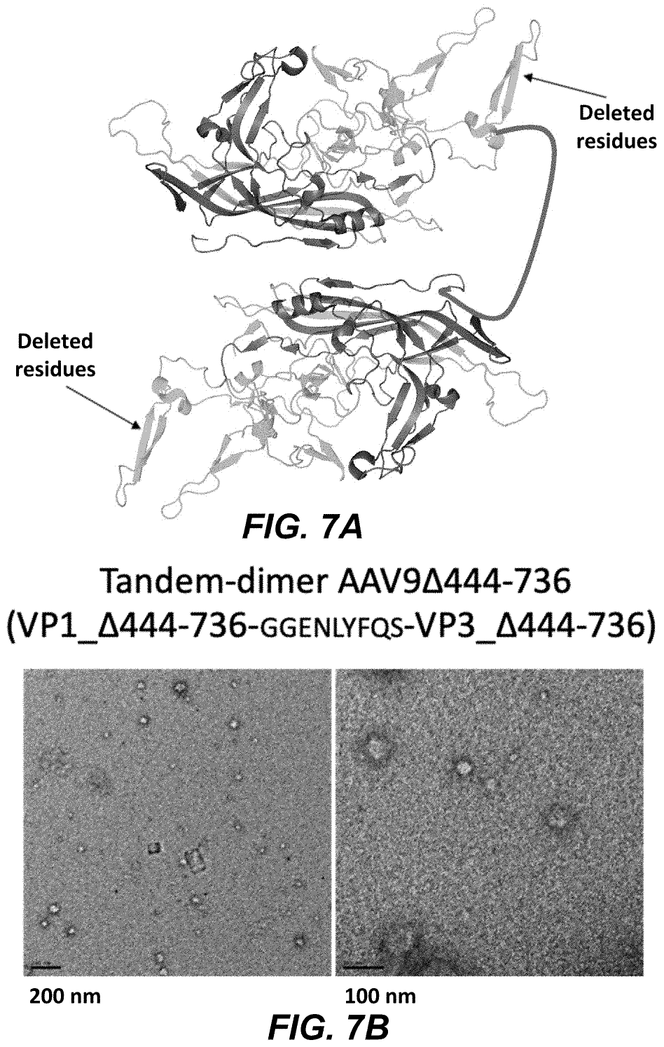

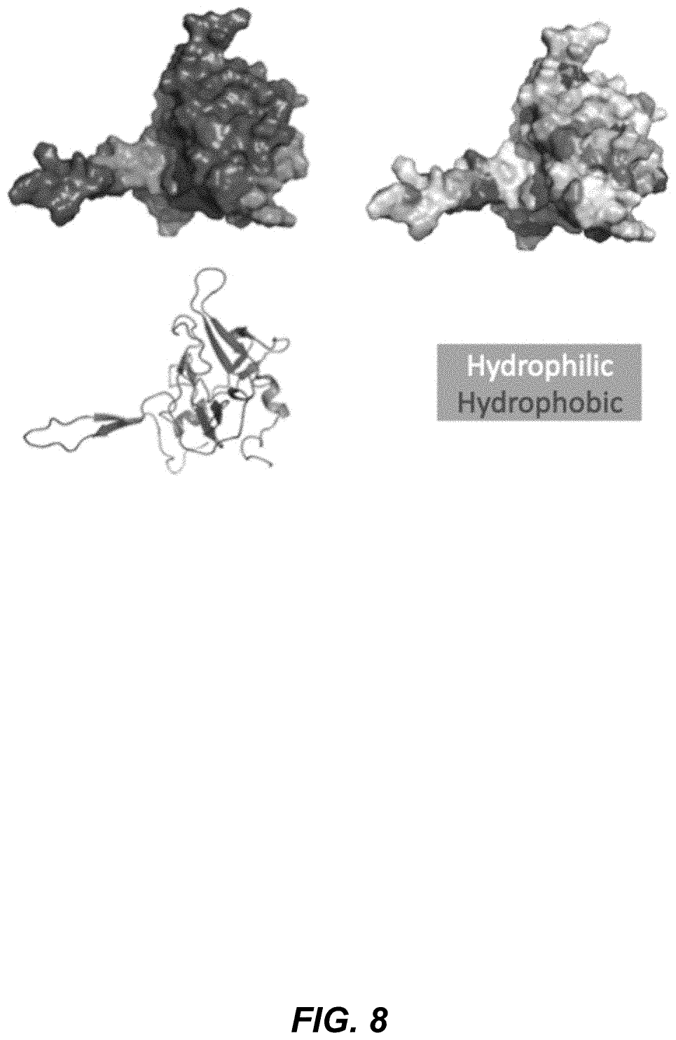

One strategy to enhance the stability of the capsids and restore the capsid forming capability is by covalently linking truncated capsid proteins. Without being bound by any particular theory, this linkage can stabilize the interaction between the two linked monomers, thus improving the overall stability of the final assembly product. Indeed, some of these dimeric capsid subunits did yield well-separated spherical particles (FIG. 7A-FIG. 7B).

The Spike Block, Forming Most 3-Fold Interactions, is a Determinant Factor for Capsid Size

Although C-terminal-truncated capsid proteins can form functional assemblies to some extent, an ideal capsid design, in some embodiments, should make as little deletion from the native sequence as possible because the evolutionarily selected sequences can be structurally and functionally important. Since the most significant difference between Δ450-736 capsid and wtAAV capsid is the loss of intertwined 3-fold interactions in the spike block, only residues that are involved in the intertwined 3-fold interactions were specifically removed and a flexible G/S linker was used to fill in the resulting gap between the bordering residues. The fact that the residues in this block had been reported to be tolerable to mutations and was predicted to be fold into a likely soluble domain with hydrophilic surfaces show that, in some embodiments, whole-domain deletions within this region can still form capsids.

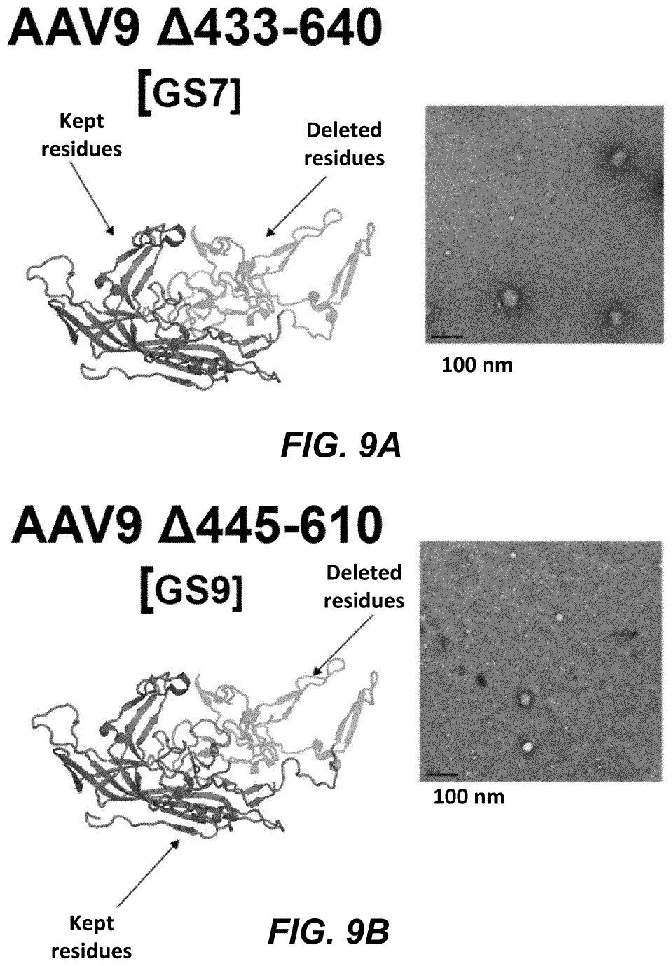

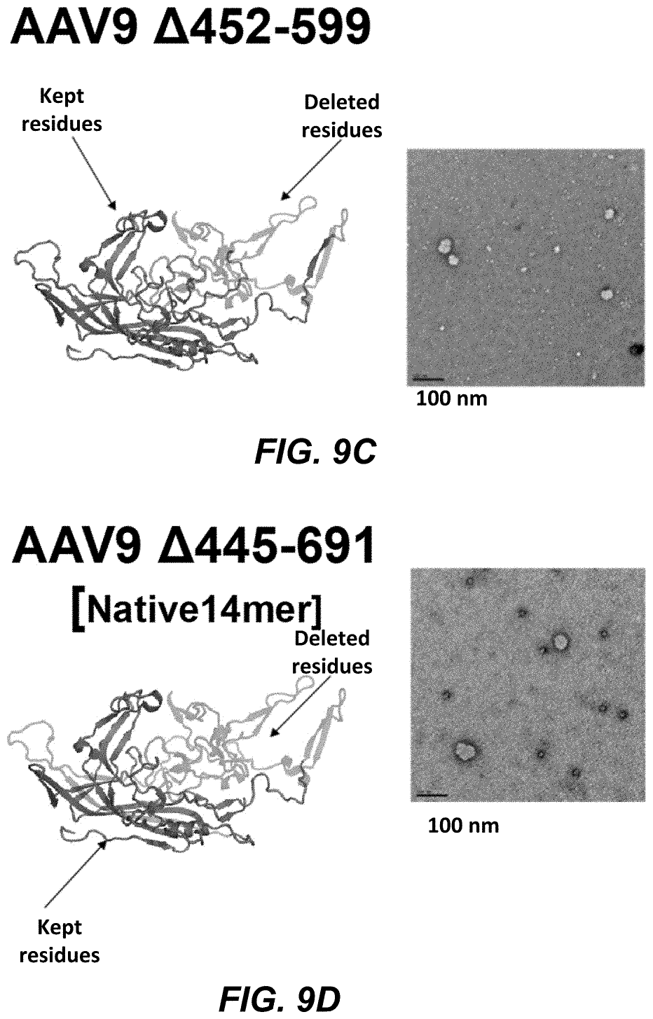

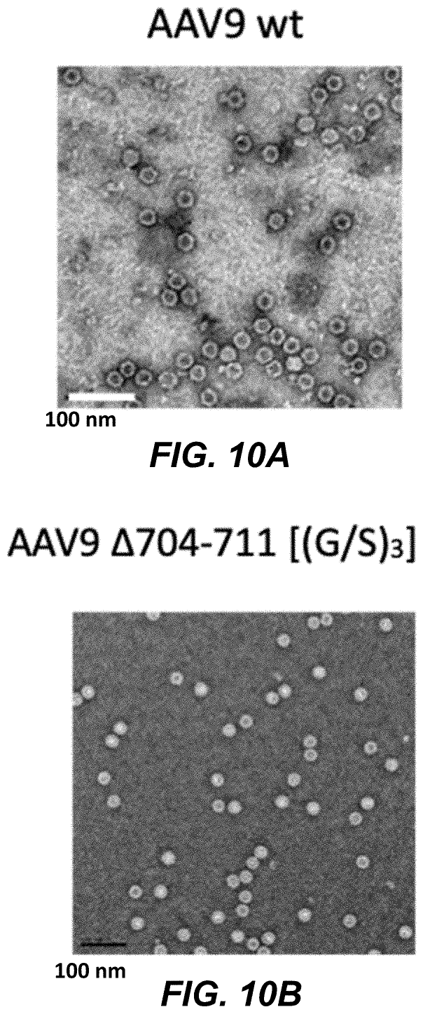

With a number of attempts, a few spike-truncated designs were identified that yielded capsids and enhanced yield of genome-protection titers when packaging an oversized genome. Interestingly, the particle size of such variants is mostly around 30-50 nm, close to the expected size of a T=3 capsid. A few examples include variants that deleted the whole spike except the N-terminal “base” (AAV9 Δ433-640 GS7) (FIG. 9A) and variants that deleted the “tip” segment (AAV9 Δ445-610 GS9 and AAV9 Δ452-599, the former used a flexible linker to link the two “arms”, while the latter effectively used native AAV9 sequences to link the two “arms”) (FIG. 9B-FIG. 9C).

Further deletion of the auxiliary segment of the core block can also yield spherical capsids (FIG. 9D), supporting the previous conclusion that the primary core block is essential for forming genome-protecting structures.

Short Deletions in the Sealer Block do not Cause a Change in Capsid Size and Capacity, and Longer Deletions in the Block can Induce Capsid Size Expansion

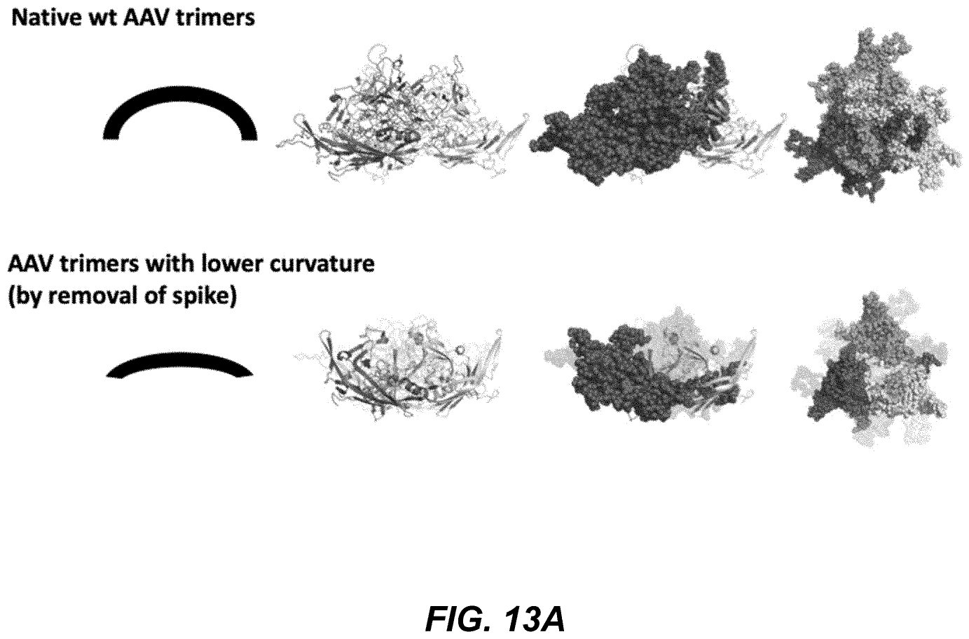

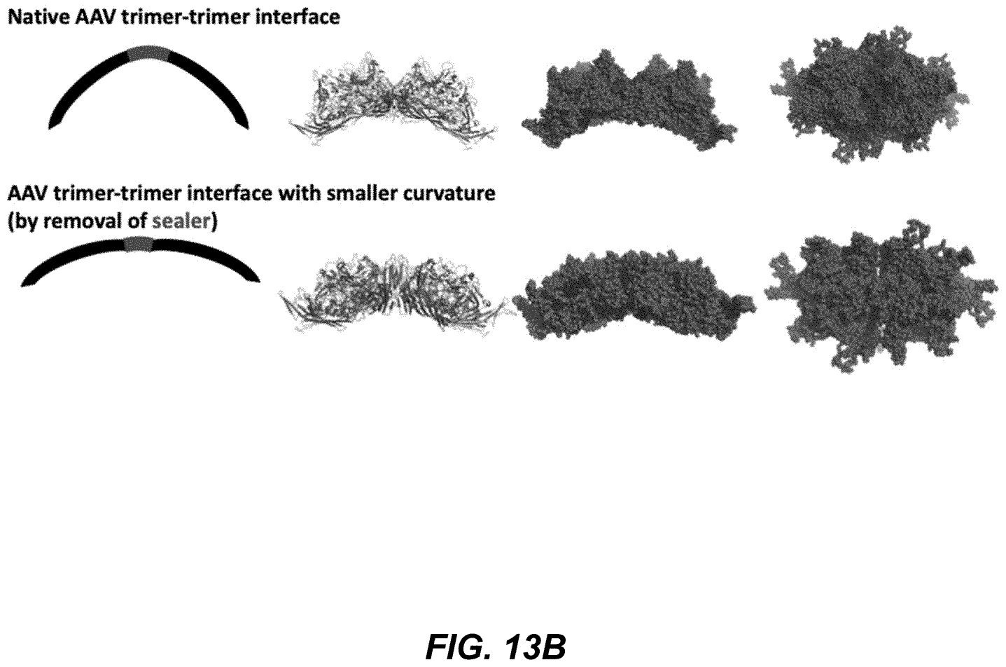

The sealer block was also missing in the Δ450-736-like variants. However, this block mainly forms a “caulk”-like structure on the surface of the capsid. The major structural function of this block can be to stabilize and fix the angles of interactions between neighboring trimers after they attach to each other (FIG. 1B, FIG. 13B).

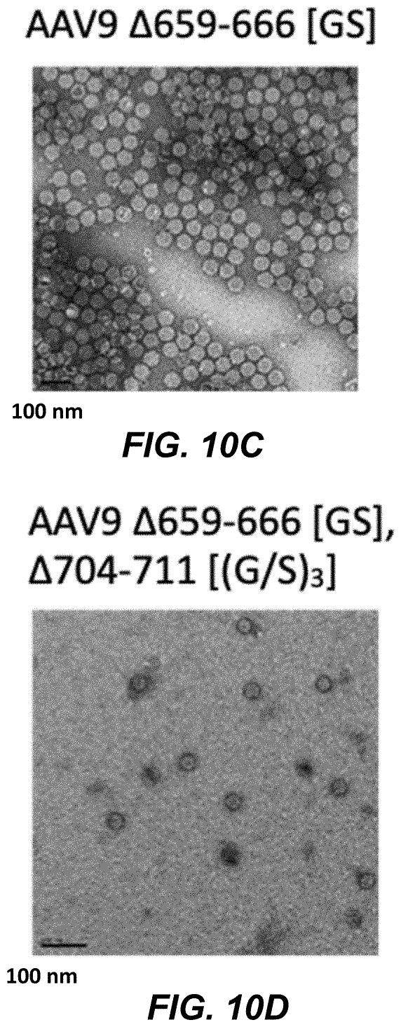

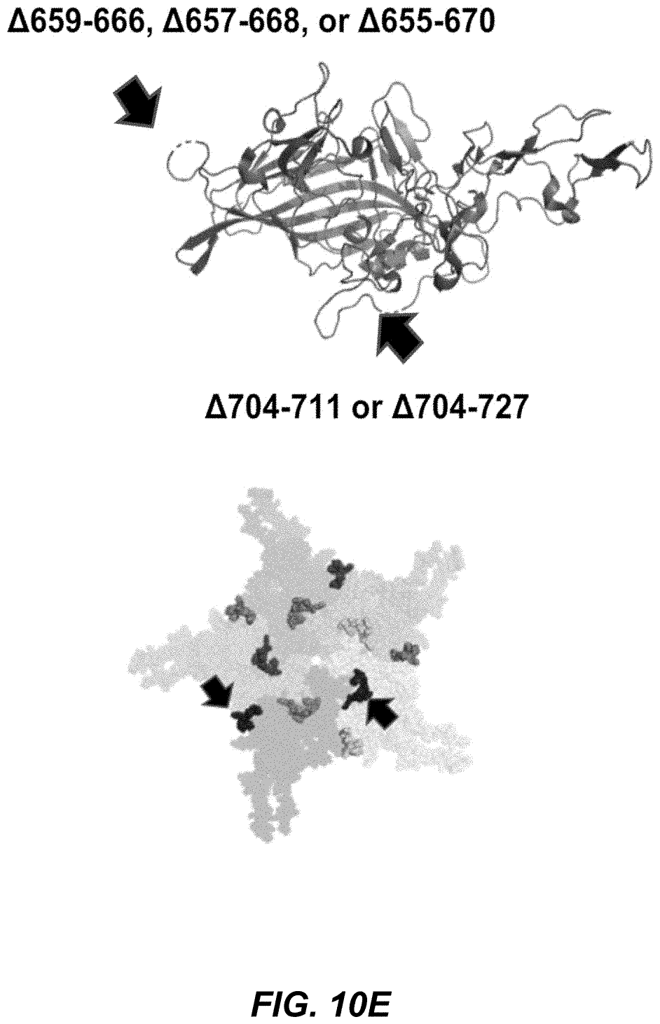

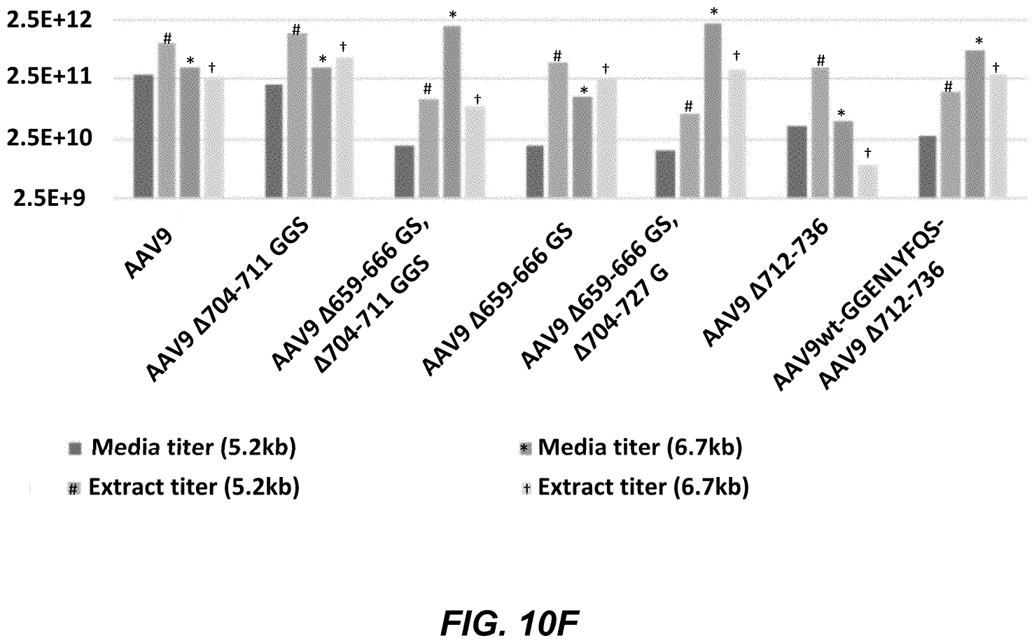

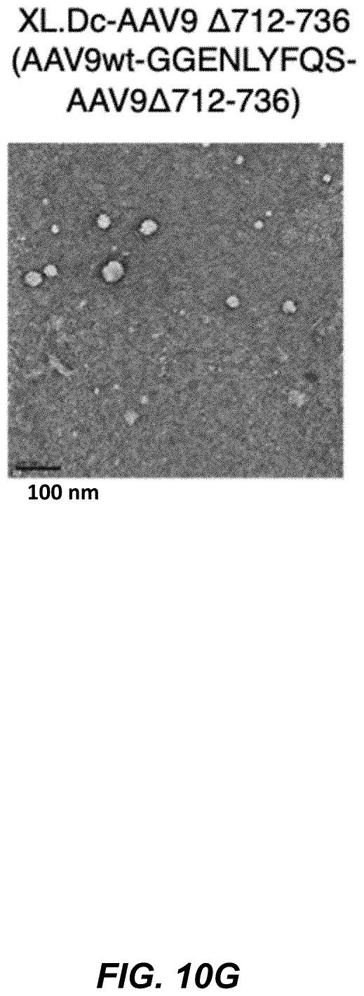

Rational deletion of loops within the sealer block still produced 25-nm, T=1 capsids (FIG. 10A-FIG. 10D) that are indistinguishable from the wild-type capsids. These T=1-forming capsids with weakened 5-fold interactions (FIG. 10E) yielded DNaseI-protected titer that is comparable to, and in some cases higher than, wild-type AAV, especially when packaging oversized genomes (FIG. 10F). The unique features of the sealer-truncated capsids do give them the ability of being developed into delivery vectors. For example, the high media titer of these variants can be taken advantage of. Moreover, these capsids can be developed into delivery vehicles for fragmented oversized genomes.

The properties of sealer-deletion variants seem to change when a larger portion of the block is truncated. Particularly, the carrying capacity of these variants can be increased. For example, AAV9 Δ659-666GS, Δ704-727 capsid produced significantly higher titer when packaging an oversized genome (FIG. 10F).

Moreover, although a C-terminal deletion variant AAV9 Δ712-736 capsid itself is, in some embodiments, a poor producer (FIG. 4A), its yield can be improved by linking it to a copy of wild-type AAV9 subunit, making a tandem-dimer unit (AAV9 wt-GGENLYFQS-AAV9Δ712-736, or XL.Dc-AAV9 Δ712-736). Such units can produce spherical capsids with heterogeneous sizes (with some larger than 30 nm) (FIG. 10G).

“Hinges” Between Blocks and Segments can be Engineered to Improve Assembly/Transduction Efficiencies of Some Capsid Variants

The “hinges” in the spike block provide flexibility and limit the physical distance between more rigid segments during the dynamic assembly of the capsid proteins. Without being bound by any particular theory, tuning the flexibility and length of such hinges can improve the assembly efficiency, transduction efficiency, and stability of some AAV variants.

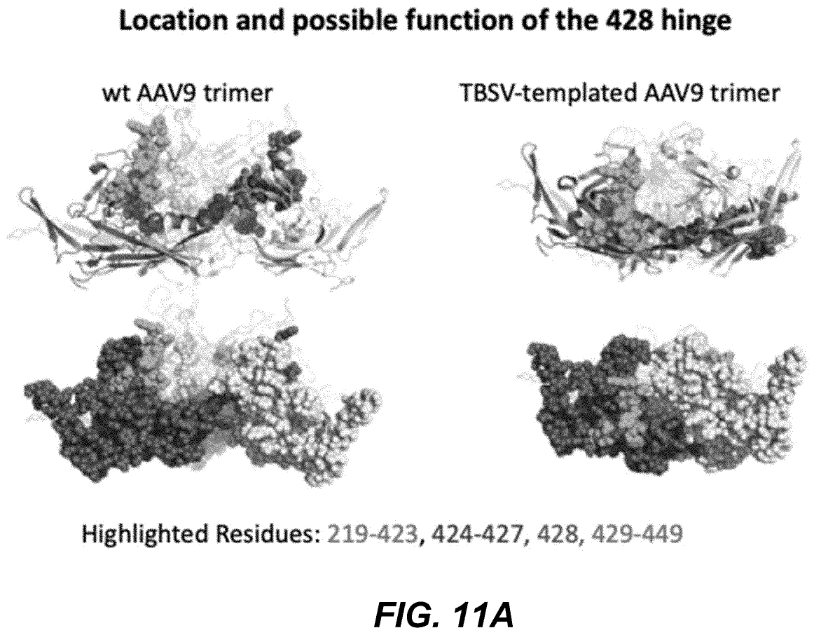

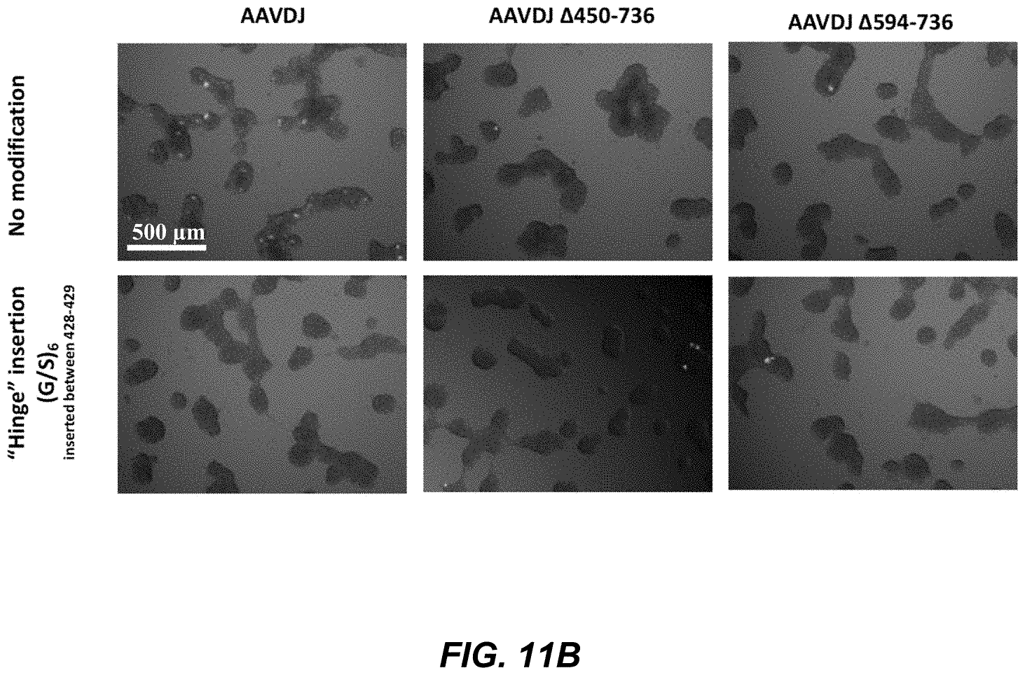

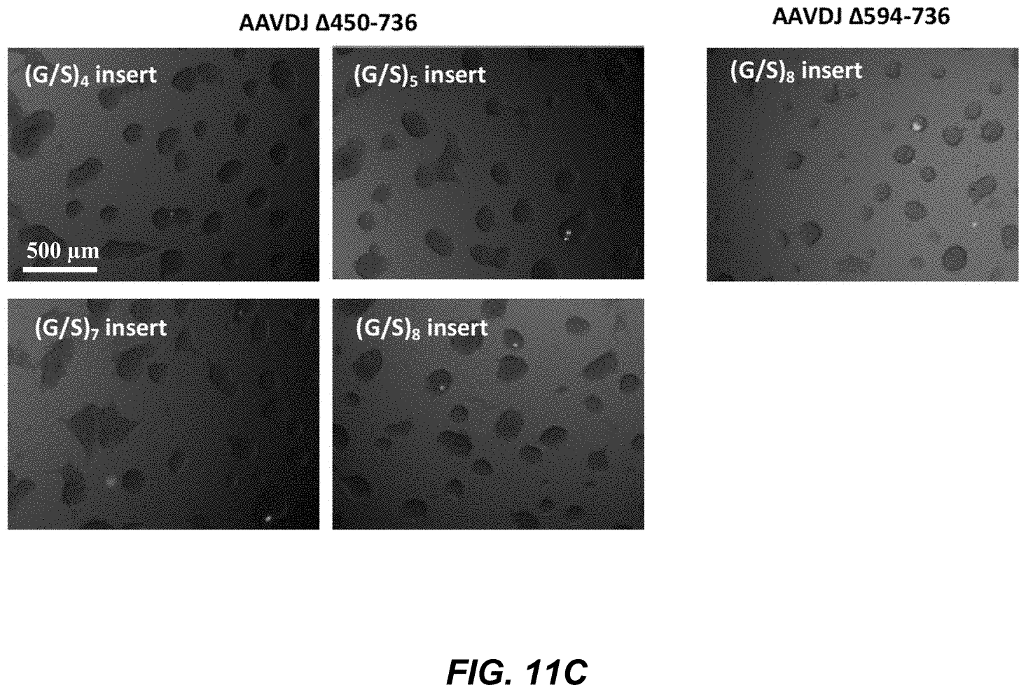

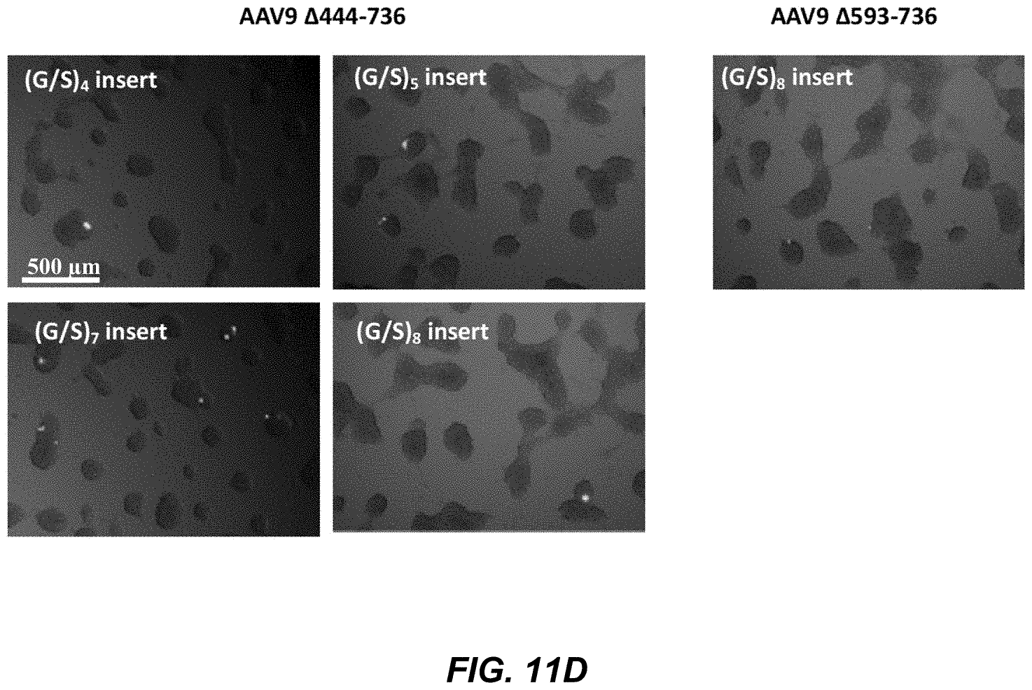

As a proof-of-principle test, one “hinge” site that appears to be critical for forming size-expanded capsids was identified and engineered. By analyzing the modeled structure of trimers, one “hinge” was predicted to be over-stretched when the capsid subunits form a “flatter” trimer (FIG. 11A), which is needed for forming capsids with an expanded radius of curvature. The site is the “hinge” linking the core block and a 3-fold interaction arm, or the “428 hinge” (residues 424-428).

Extending the hinge region with an inserted flexible peptide indeed improves (FIG. 11B) the infectious titer of the C-terminal truncated capsids, especially the Δ450-736-like capsids. The optimal length of the extra peptide for a Δ450-736-like capsid is around 7 aa (FIG. 11C-FIG. 11D) in both an AAV-DJ backbone or an AAV9 backbone. In some embodiments, when extending the 428 hinge, insertion is a preferred method over substitution, as all substitution variants that removed the conserved triple residues 426-YAH-428 are no longer infectious.

Without being bound by any particular theory, such “hinge” modifications can also be applied to other “hinges” listed in Table 5.

Capsid Designs Guided by the Understanding of the Structural Dissection Yields Capsid Variants that can Transduce Oversized Genomes

Combining the obtained knowledge by studying each block individually, a few variants with the goal to minimize removal of residues involved in receptor binding (for example residues involved in galactose binding) were rationally designed, while still achieving the goal of surface curvature reduction.

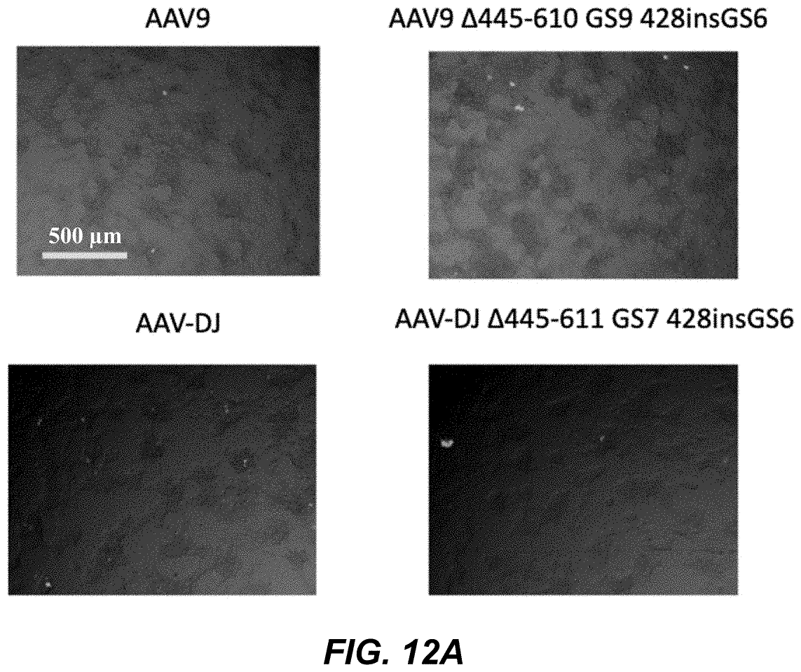

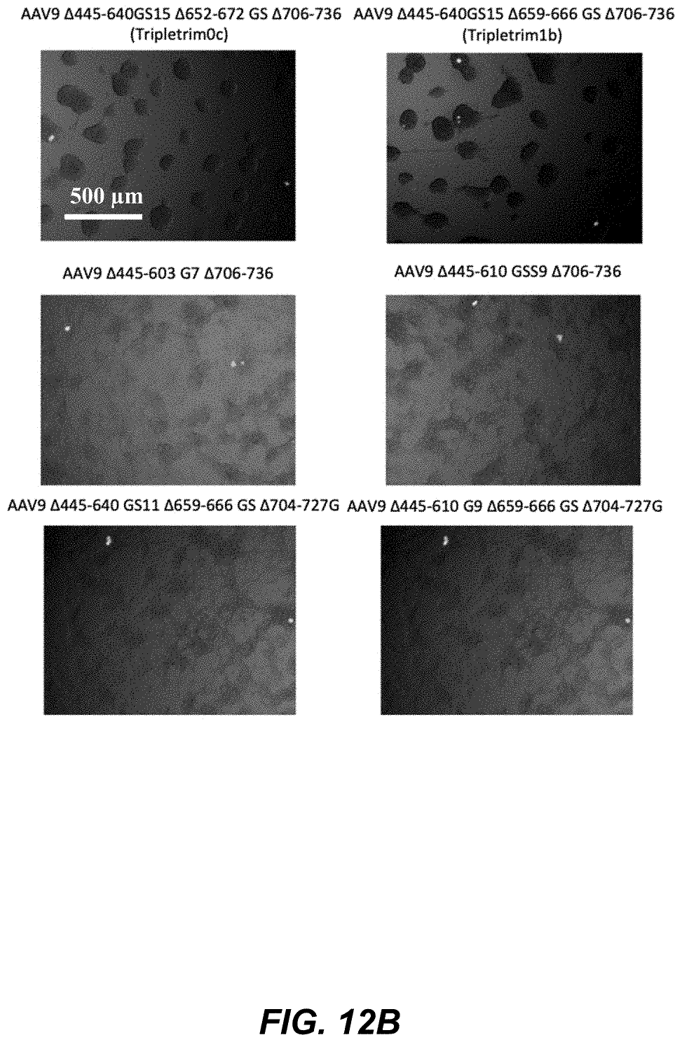

The infectious titer of a few successful variants is shown in FIG. 12A-FIG. 12B. One group of variants has both a deletion in the spike block and an insertion at the “428 hinge” (FIG. 12A). Another group of variants has truncations in both the spike block and the sealer block (FIG. 12B). The improved infectious titer indicates an increased packaging efficiency and/or transduction efficiency of an oversized (6.1 kb) cargo.

Based on the systematic truncations of an AAV capsid protein, the size-determining factor for AAV capsid assembly resides, in some instances, in the 3-fold interacting spike region and the 2-fold interacting sealer block. When truncated, they can produce capsids with increased sizes. Most of the size-expanded capsids can package and protect oversized genomes. Although, in some embodiments, some of such deletions cause decreased homogeneity and infectivity, these properties can be improved by further engineering.

Without being bound by any particular theory, one mechanism of the size expansion is through reduction of the surface curvature. To elaborate, certain truncations within the spike block can help reduce the spatial constraints at the center of an AAV trimer and allow the trimer to be “flatter” e.g., reducing the intra-trimer curvature (FIG. 13A). In some embodiments, long truncations in the sealer block can reduce the force bending two neighboring trimers, thus reducing the inter-trimer curvature (FIG. 13B)

Although AAV capsid variants with specific deletions within the spike region have produced size-expanded capsids with decent titers, they still suffer from relatively low transduction efficiency. In some embodiments, one reason is that the newly exposed capsid protein surfaces are not optimized for solubility and new types of inter-subunit interactions. In some embodiments, another reason for the loss in transduction efficiency is the loss of residues in the 3-fold spike that are necessary for virus-receptor interactions.

Without being bound by any particular theory, fine-tuning the truncation sites can improve the yield of these variants. Based on the structural dissection, some embodiments include:

Design group 1: Deletion of the “arm” and “tip” (e.g., AAV9 Δ429-607GS4), with part of the sealer group optionally removed;

Design group 2: Deletion of the full spike block (e.g., AAV9 Δ418-640GS3), with part of the sealer group optionally removed;

Design group 3: Deletion of the full spike block except the “base” segment before the 428 hinge (e.g., AAV9 Δ428-640GS9), with part of the sealer group optionally removed.

Without being bound by any particular theory, supplementing the full spike block back to these capsids can also improve the infectivity of the spike-deleted variants. There are a few strategies, including:

Strategy 1: to construct full-length capsid subunits with flexible linkers inserted between the blocks or the segments. For example, instead of deleting a part of the spike sequence, long, flexible linkers (e.g., the (G/S)8 linkers) can be inserted at boundaries of the target sequence to “separate” the folding processes of the separated blocks or segments. Insertion sites for these linkers can be boundaries between the blocks (e.g. the core-spike boundaries around the 417th residue and the 640th residue), the boundaries between key segments (e.g., the “arm”-“tip” boundary around the 445th residue and the “tip”-“base” boundary around the 604th residue), and/or the “hinges” specified in Table 5. This can allow independent folding of the core blocks and the spike block, thus reducing steric hindrance against a reduction of curvature within a trimer. Similarly, flexible linkers can be added between the core block and the sealer block to reduce steric hindrance against a reduction of curvature between trimers (FIG. 13A-FIG. 13B).

Strategy 2: to make tandem dimer units of a deletion-bearing variant and another variant with “complementary” residues (e.g., a wt capsid protein), for example like the variant shown in FIG. 10G.

Strategy 3: to swap the spike block to the C-terminal end of the capsid protein. Since the C-terminus of wtAAV (e.g., 736th residue in AAV9 VP1) is close to the boundary between the “base” and “arm” of the spike block, it is reasonable to “add back” the partially or completely deleted spike-block sequence by inserting it at the end of the capsid protein, optionally with a flexible linker.

Strategy 4: to make mosaic capsids by co-expressing a deletion-bearing variant and another variant with “complementary” residues (e.g., a wt capsid protein). When carefully designed, some of these mosaic capsids can possess both the expanded sizes and functional residues needed for stability and successful gene delivery.

Strategy 5: to add the deleted sequence back in trans. For example, to supplement a spike-block-deleted variant, it is possible to express part of the spike block (for example, residues 445-610, residues 429-607, residues 418-640, residues 428-640, or a similar fragment) alone from a separate plasmid during the production. Given the abundance of interactions between the 3-fold spike and the “platform”, these peptides will self-assemble.

Finally, well-established directed evolution technologies can be used to optimize the new surface/interface residues for improved capsid stability, packaging efficiency, and transduction efficiency.

The capsid designs showing expanded sizes described herein can provide foundational AAV vectors with increased cargo capacity with applications in gene therapy and provide an exemplar for the rational expansion of protein nanocages. These size-expanded AAV capsids, can serve as delivery tools for larger genetic cargos and enable novel gene therapies.

All sequences tested in Example 1 are listed in Table 6 below:

TABLE 6
EXEMPLARY PEPTIDE SEQUENCES TESTED

| | Name | | | | |
|---|---|---|---|---|---|
| SEQ | (default | | | | Peptide sequence (starting |
| ID | backbone: | Other | Design | Observed | with the 219th or the 220th |
| NO | AAV9) | names | rationale | phenotypes | residues in AAV9 or AAV-DJ VP1) |
| 1 | AAV9 Δ426- | N/A | C-terminal | Forms genome- | DGVGSSSGNWHCDSQWLGDRVITTSTRT |
| | 736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLNFKLFNIQVKEVTDNNG |
| | | | | heterogeneous | VKTIANNLTSTVQVFTDSDYQLPYVLGSA |
| | | | | sizes. Tends to | HEGCLPPFPADVFMIPQYGYLTLNDGSQA |
| | | | | aggregate. AF2- | VGRSSFYCLEYFPSQMLRTGNNFQFSYEF |
| | | | | predicted to form | ENVPFHSS |
| | | | | homohexamers. | |
| 2 | AAV9 Δ444- | N/A | C-terminal | Forms genome- | DGVGSSSGNWHCDSQWLGDRVITTSTRT |
| | 736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLNFKLFNIQVKEVTDNNG |
| | | | | heterogeneous | VKTIANNLTSTVQVFTDSDYQLPYVLGSA |
| | | | | sizes. Tends to | HEGCLPPFPADVFMIPQYGYLTLNDGSQA |
| | | | | aggregate. | VGRSSFYCLEYFPSQMLRTGNNFQFSYEF |
| | | | | | ENVPFHSSYAHSQSLDRLMNPLIDQY |
| 3 | AAV9 Δ450- | N/A | C-terminal | Forms genome- | DGVGSSSGNWHCDSQWLGDRVITTSTRT |
| | 736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLNFKLFNIQVKEVTDNNG |
| | | | | heterogeneous | VKTIANNLTSTVQVFTDSDYQLPYVLGSA |
| | | | | sizes. Tends to | HEGCLPPFPADVFMIPQYGYLTLNDGSQA |
| | | | | aggregate. | VGRSSFYCLEYFPSQMLRTGNNFQFSYEF |
| | | | | | ENVPFHSSYAHSQSLDRLMNPLIDQYLYY |
| | | | | | LSK |
| 4 | AAV9 Δ452- | N/A | C-terminal | Forms genome- | DGVGSSSGNWHCDSQWLGDRVITTSTRT |
| | 736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLNFKLFNIQVKEVTDNNG |
| | | | | heterogeneous | VKTIANNLTSTVQVFTDSDYQLPYVLGSA |
| | | | | sizes. Tends to | HEGCLPPFPADVFMIPQYGYLTLNDGSQA |
| | | | | aggregate. | VGRSSFYCLEYFPSQMLRTGNNFQFSYEF |
| | | | | | ENVPFHSSYAHSQSLDRLMNPLIDQYLYY |
| | | | | | LSKTI |
| 5 | AAV9 Δ593- | N/A | C-terminal | Forms genome- | DGVGSSSGNWHCDSQWLGDRVITTSTRT |
| | 736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLNFKLFNIQVKEVTDNNG |
| | | | | heterogeneous | VKTIANNLTSTVQVFTDSDYQLPYVLGSA |
| | | | | sizes. Tends to | HEGCLPPFPADVFMIPQYGYLTLNDGSQA |
| | | | | aggregate. | VGRSSFYCLEYFPSQMLRTGNNFQFSYEF |
| | | | | | ENVPFHSSYAHSQSLDRLMNPLIDQYLYY |
| | | | | | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQ |
| 6 | AAV9 Δ410- | A+Dt | Core- | Forms genome- | DGVGSSSGNWHCDSQWLGDRVITTSTRT |
| | 653 GGS | | block-only | protecting, size- | WALPTYNNHLYKQISNSTSGGSSNDNAY |
| | Δ691-736 | | variant | expanded, | FGYSTPWGYFDFNRFHCHFSPRDWQRLI |
| | | | | spherical capsids. | NNNWGFRPKRLNFKLFNIQVKEVTDNNG |
| | | | | | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNGGSVPADP |
| | | | | | PTAFNKDKLNSFITQYSTGQVSVEIEWEL |
| | | | | | QKEG |
| 7 | AAV9 | Tandem- | Two C- | Forms genome- | DGVGSSSGNWHCDSQWLGDRVITTSTRT |
| | VP1Δ444- | dimer | terminal | protecting, size- | WALPTYNNHLYKQISNSTSGGSSNDNAY |
| | 736- | AAV9Δ444- | truncated | expanded, | FGYSTPWGYFDFNRFHCHFSPRDWQRLI |
| | GGENLYFQS- | 736; | subunits | spherical capsids | NNNWGFRPKRLNFKLFNIQVKEVTDNNG |
| | VP3Δ444- | XL.Dc- | tandemly | that are infectious | VKTIANNLTSTVQVFTDSDYQLPYVLGSA |
| | 736 | Δ444 | linked by | | HEGCLPPFPADVFMIPQYGYLTLNDGSQA |
| | | | a TEV | | VGRSSFYCLEYFPSQMLRTGNNFQFSYEF |
| | | | protease | | ENVPFHSSYAHSQSLDRLMNPLIDQYENL |
| | | | cleavage | | YFQGASGGGAPVADNNEGADGVGSSSG |
| | | | site | | NWHCDSQWLGDRVITTSTRTWALPTYN |
| | | | | | NHLYKQISNSTSGGSSNDNAYFGYSTPW |
| | | | | | GYFDFNRFHCHFSPRDWQRLINNNWGFR |
| | | | | | PKRLNFKLFNIQVKEVTDNNGVKTIANNL |
| | | | | | TSTVQVFTDSDYQLPYVLGSAHEGCLPPF |
| | | | | | PADVFMIPQYGYLTLNDGSQAVGRSSFY |
| | | | | | CLEYFPSQMLRTGNNFQFSYEFENVPFHS |
| | | | | | SYAHSQSLDRLMNPLIDQY |
| 8 | AAVDJ | N/A | C-terminal | Forms genome- | GVGNSSGNWHCDSTWMGDRVITTSTRT |
| | Δ450-736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLSFKLFNIQVKEVTQNEG |
| | | | | heterogeneous | TKTIANNLTSTIQVFTDSEYQLPYVLGSA |
| | | | | sizes. Tends to | HQGCLPPFPADVFMIPQYGYLTLNNGSQ |
| | | | | aggregate. | AVGRSSFYCLEYFPSQMLRTGNNFQFTYT |
| | | | | | FEDVPFHSSYAHSQSLDRLMNPLIDQYLY |
| | | | | | YLSR |
| 9 | AAVDJ | N/A | C-terminal | Forms genome- | GVGNSSGNWHCDSTWMGDRVITTSTRT |
| | Δ594-736 | | truncation | protecting | WALPTYNNHLYKQISNSTSGGSSNDNAY |
| | | | | spherical capsids | FGYSTPWGYFDFNRFHCHFSPRDWQRLI |
| | | | | with | NNNWGFRPKRLSFKLFNIQVKEVTQNEG |
| | | | | heterogeneous | TKTIANNLTSTIQVFTDSEYQLPYVLGSA |
| | | | | sizes. Tends to | HQGCLPPFPADVFMIPQYGYLTLNNGSQ |
| | | | | aggregate. | AVGRSSFYCLEYFPSQMLRTGNNFQFTYT |
| | | | | | FEDVPFHSSYAHSQSLDRLMNPLIDQYLY |
| | | | | | YLSRTQTTGGTTNTQTLGFSQGGPNTMA |
| | | | | | NQAKNWLPGPCYRQQRVSKTSADNNNS |
| | | | | | EYSWTGATKYHLNGRDSLVNPGPAMAS |
| | | | | | HKDDEEKFFPQSGVLIFGKQGSEKTNVDI |
| | | | | | EKVMITDEEEIRTTNPVATEQYGSVSTNL |
| | | | | | QRGNRQAA |
| 10 | AAV9 Δ433- | Δ433-640 | deleted the | Forms genome- | DGVGSSSGNWHCDSQWLGDRVITTSTRT |
| | 640 GS7 | G7; | whole | protecting | WALPTYNNHLYKQISNSTSGGSSNDNAY |
| | | Δ433-640 | spike | spherical capsid | FGYSTPWGYFDFNRFHCHFSPRDWQRLI |
| | | [(G/S)7] | except the | with expanded | NNNWGFRPKRLNFKLFNIQVKEVTDNNG |
| | | | N-terminal | sizes. | VKTIANNLTSTVQVFTDSDYQLPYVLGSA |
| | | | “base” | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLGGSGGGSKHPPPQI |
| | | | | | LIKNTPVPADPPTAFNKDKLNSFITQYSTG |
| | | | | | QVSVEIEWELQKENSKRWNPEIQYTSNY |
| | | | | | YKSNNVEFAVNTEGVYSEPRPIGTRYLTR |
| | | | | | NL |
| 11 | AAV9 Δ433- | Δ433-605 | Deletion | Forms genome- | DGVGSSSGNWHCDSQWLGDRVITTSTRT |
| | 605 GS7 | G7; | between | protecting | WALPTYNNHLYKQISNSTSGGSSNDNAY |
| | | Δ433-605 | hinges in | spherical capsid | FGYSTPWGYFDFNRFHCHFSPRDWQRLI |
| | | [(G/S)7] | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLGGSGGGSVWQDRD |
| | | | | | VYLQGPIWAKIPHTDGNFHPSPLMGGFG |
| | | | | | MKHPPPQILIKNTPVPADPPTAFNKDKLN |
| | | | | | SFITQYSTGQVSVEIEWELQKENSKRWNP |
| | | | | | EIQYTSNYYKSNNVEFAVNTEGVYSEPRP |
| | | | | | IGTRYLTRNL |
| 12 | AAV9 Δ445- | Δ445-610 | Deletion | Forms genome- | DGVGSSSGNWHCDSQWLGDRVITTSTRT |
| | 610 GS9 | G9; | between | protecting | WALPTYNNHLYKQISNSTSGGSSNDNAY |
| | | Δ445-610 | hinges in | spherical capsid | FGYSTPWGYFDFNRFHCHFSPRDWQRLI |
| | | [(G/S)9] | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGSDVYLQGPIWAKIPHTDGNFHPS |
| | | | | | PLMGGFGMKHPPPQILIKNTPVPADPPTA |
| | | | | | FNKDKLNSFITQYSTGQVSVEIEWELQKE |
| | | | | | NSKRWNPEIQYTSNYYKSNNVEFAVNTE |
| | | | | | GVYSEPRPIGTRYLTRNL |
| 13 | AAV9 Δ452- | N/A | Deletion | Forms genome- | DGVGSSSGNWHCDSQWLGDRVITTSTRT |
| | 599 | | between | protecting | WALPTYNNHLYKQISNSTSGGSSNDNAY |
| | | | hinges in | spherical capsid | FGYSTPWGYFDFNRFHCHFSPRDWQRLI |
| | | | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSKTIGILPGMVWQDRDVYLQGPIWAKIP |
| | | | | | HTDGNFHPSPLMGGFGMKHPPPQILIKNT |
| | | | | | PVPADPPTAFNKDKLNSFITQYSTGQVSV |
| | | | | | EIEWELQKENSKRWNPEIQYTSNYYKSN |
| | | | | | NVEFAVNTEGVYSEPRPIGTRYLTRNL |
| 14 | AAV9 Δ445- | AAV9 | Deletion | Forms genome- | DGVGSSSGNWHCDSQWLGDRVITTSTRT |
| | 691 | Δ445- | between | protecting | WALPTYNNHLYKQISNSTSGGSSNDNAY |
| | insNative | 464, | hinges in | spherical capsid | FGYSTPWGYFDFNRFHCHFSPRDWQRLI |
| | 14 mer | Δ479-691 | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLVA |
| | | | | | GPSNMAVQGRNYSKRWNPEIQYTSNYY |
| | | | | | KSNNVEFAVNTEGVYSEPRPIGTRYLTRN |
| | | | | | L |
| 15 | AAV9 Δ445- | N/A | Deletion | Forms genome- | DGVGSSSGNWHCDSQWLGDRVITTSTRT |
| | 603G9 | | between | protecting | WALPTYNNHLYKQISNSTSGGSSNDNAY |
| | | | hinges in | spherical capsid | FGYSTPWGYFDFNRFHCHFSPRDWQRLI |
| | | | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGSGMVWQDRDVYLQGPIWAKIP |
| | | | | | HTDGNFHPSPLMGGFGMKHPPPQILIKNT |
| | | | | | PVPADPPTAFNKDKLNSFITQYSTGQVSV |
| | | | | | EIEWELQKENSKRWNPEIQYTSNYYKSN |
| | | | | | NVEFAVNTEGVYSEPRPIGTRYLTRNL |
| 16 | AAV9 Δ417- | N/A | Deletion | Forms genome- | DGVGSSSGNWHCDSQWLGDRVITTSTRT |
| | 640 | | between | protecting | WALPTYNNHLYKQISNSTSGGSSNDNAY |
| | | | hinges in | spherical capsid | FGYSTPWGYFDFNRFHCHFSPRDWQRLI |
| | | | spike | with expanded | NNNWGFRPKRLNFKLFNIQVKEVTDNNG |
| | | | block | sizes. | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEK |
| | | | | | HPPPQILIKNTPVPADPPTAFNKDKLNSFIT |
| | | | | | QYSTGQVSVEIEWELQKENSKRWNPEIQ |
| | | | | | YTSNYYKSNNVEFAVNTEGVYSEPRPIGT |
| | | | | | RYLTRNL |
| 17 | AAV9 Δ658- | AAV9 d6 | sealer | Forms T = 1 | DGVGSSSGNWHCDSQWLGDRVITTSTRT |
| | 667 GS | | domain | capsids | WALPTYNNHLYKQISNSTSGGSSNDNAY |
| | | | deletion | | FGYSTPWGYFDFNRFHCHFSPRDWQRLI |
| | | | | | NNNWGFRPKRLNFKLFNIQVKEVTDNNG |
| | | | | | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSRTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPPTAFNKDKLNSFIT |
| | | | | | QYSTGQVSVEIEWELQKENSKRWNPEIQ |
| | | | | | YTSNYYKSNNVEFAVNTEGVYSEPRPIGT |
| | | | | | RYLTRNL |
| 18 | AAV9 Δ704- | AAV9 | sealer | Forms T = 1 | DGVGSSSGNWHCDSQWLGDRVITTSTRT |
| | 711 GS3 | Δ704-711 | domain | capsids | WALPTYNNHLYKQISNSTSGGSSNDNAY |
| | | (G/S)₃ | deletion | | FGYSTPWGYFDFNRFHCHFSPRDWQRLI |
| | | | | | NNNWGFRPKRLNFKLFNIQVKEVTDNNG |
| | | | | | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSRTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPPTAFNKDKLNSFIT |
| | | | | | QYSTGQVSVEIEWELQKENSKRWNPEIQ |
| | | | | | YTSGGSEFAVNTEGVYSEPRPIGTRYLTR |
| | | | | | NL |
| 19 | AAV9 Δ659- | AAV9 | sealer | Forms T = 1 | DGVGSSSGNWHCDSQWLGDRVITTSTRT |
| | 666 GS, | Δ659-666 | domain | capsids | WALPTYNNHLYKQISNSTSGGSSNDNAY |
| | Δ704-711 | GS, | deletion | | FGYSTPWGYFDFNRFHCHFSPRDWQRLI |
| | GS3 | Δ704-711 | | | NNNWGFRPKRLNFKLFNIQVKEVTDNNG |
| | | (G/S)3 | | | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSRTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPGSLNSFITQYSTGQ |
| | | | | | VSVEIEWELQKENSKRWNPEIQYTSGGSE |
| | | | | | FAVNTEGVYSEPRPIGTRYLTRNL |
| 20 | AAV9 Δ704- | N/A | sealer | Packages | DGVGSSSGNWHCDSQWLGDRVITTSTRT |
| | 727 G | | domain | oversized | WALPTYNNHLYKQISNSTSGGSSNDNAY |
| | | | deletion | genomes more | FGYSTPWGYFDFNRFHCHFSPRDWQRLI |
| | | | | efficiently than | NNNWGFRPKRLNFKLFNIQVKEVTDNNG |
| | | | | regular-sized | VKTIANNLTSTVQVFTDSDYQLPYVLGSA |
| | | | | genomes. | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPPTAFNKDKLNSFIT |
| | | | | | QYSTGQVSVEIEWELQKENSKRWNPEIQ |
| | | | | | YTSGGTRYLTRNL |
| 21 | AAV9 Δ712- | N/A | sealer | Do not package | DGVGSSSGNWHCDSQWLGDRVITTSTRT |
| | 736 | | domain | genome | WALPTYNNHLYKQISNSTSGGSSNDNAY |
| | | | deletion | efficiently. | FGYSTPWGYFDFNRFHCHFSPRDWQRLI |
| | | | | | NNNWGFRPKRLNFKLFNIQVKEVTDNNG |
| | | | | | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLYY |
| | | | | | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPPTAFNKDKLNSFIT |
| | | | | | QYSTGQVSVEIEWELQKENSKRWNPEIQ |
| | | | | | YTSNYYKSNNV |
| 22 | AAV9wt- | XL.D1cA | tandem- | Forms size- | DGVGSSSGNWHCDSQWLGDRVITTSTRT |
| | GGENLYFQS- | AV9- | dimer of a | expanded | WALPTYNNHLYKQISNSTSGGSSNDNAY |
| | AAV9Δ712- | Δ712-736 | wild type | capsids. Packages | FGYSTPWGYFDFNRFHCHFSPRDWQRLI |
| | 736 | | subunit | oversized | NNNWGFRPKRLNFKLFNIQVKEVTDNNG |
| | | | and a | genomes more | VKTIANNLTSTVQVFTDSDYQLPYVLGSA |
| | | | sealer- | efficiently than | HEGCLPPFPADVFMIPQYGYLTLNDGSQA |
| | | | deleted | regular-sized | VGRSSFYCLEYFPSQMLRTGNNFQFSYEF |
| | | | variant | genomes. | ENVPFHSSYAHSQSLDRLMNPLIDQYLYY |
| | | | | Infectious. | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGMK |
| | | | | | HPPPQILIKNTPVPADPPTAFNKDKLNSFIT |
| | | | | | QYSTGQVSVEIEWELQKENSKRWNPEIQ |
| | | | | | YTSNYYKSNNVEFAVNTEGVYSEPRPIGT |
| | | | | | RYLTRNLGGENLYFQSASGGGAPVADNN |
| | | | | | EGADGVGSSSGNWHCDSQWLGDRVITTS |
| | | | | | TRTWALPTYNNHLYKQISNSTSGGSSND |
| | | | | | NAYFGYSTPWGYFDFNRFHCHFSPRDWQ |
| | | | | | RLINNNWGFRPKRLNFKLFNIQVKEVTDN |
| | | | | | NGVKTIANNLTSTVQVFTDSDYQLPYVL |
| | | | | | GSAHEGCLPPFPADVFMIPQYGYLTLNDG |
| | | | | | SQAVGRSSFYCLEYFPSQMLRTGNNFQFS |
| | | | | | YEFENVPFHSSYAHSQSLDRLMNPLIDQY |
| | | | | | LYYLSKTINGSGQNQQTLKFSVAGPSNM |
| | | | | | AVQGRNYIPGPSYRQQRVSTTVTQNNNS |
| | | | | | EFAWPGASSWALNGRNSLMNPGPAMAS |
| | | | | | HKEGEDRFFPLSGSLIFGKQGTGRDNVDA |
| | | | | | DKVMITNEEEIKTTNPVATESYGQVATNH |
| | | | | | QSAQAQAQTGWVQNQGILPGMVWQDR |
| | | | | | DVYLQGPIWAKIPHTDGNFHPSPLMGGF |
| | | | | | GMKHPPPQILIKNTPVPADPPTAFNKDKL |
| | | | | | NSFITQYSTGQVSVEIEWELQKENSKRWN |
| | | | | | PEIQYTSNYYKSNNV |
| 23 | AAVDJ | AAVDJ | C-terminal | Forms genome- | GVGNSSGNWHCDSTWMGDRVITTSTRT |
| | Δ450-736 | Δ450-736 | truncation | protecting | WALPTYNNHLYKQISNSTSGGSSNDNAY |
| | 428insGS4 | 428ins | with | spherical capsids | FGYSTPWGYFDFNRFHCHFSPRDWQRLI |
| | | (G/S)4 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHSGGGSQSLDRLMNPLID |
| | | | | | QYLYYLSR |
| 24 | AAVDJ | AAVDJ | C-terminal | Forms genome- | GVGNSSGNWHCDSTWMGDRVITTSTRT |
| | Δ594-736 | Δ594-736 | truncation | protecting | WALPTYNNHLYKQISNSTSGGSSNDNAY |
| | 428insGS4 | 428ins | with | spherical capsids | FGYSTPWGYFDFNRFHCHFSPRDWQRLI |
| | | (G/S)4 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHSGGGSQSLDRLMNPLID |
| | | | | | QYLYYLSRTQTTGGTTNTQTLGFSQGGP |
| | | | | | NTMANQAKNWLPGPCYRQQRVSKTSAD |
| | | | | | NNNSEYSWTGATKYHLNGRDSLVNPGPA |
| | | | | | MASHKDDEEKFFPQSGVLIFGKQGSEKTN |
| | | | | | VDIEKVMITDEEEIRTTNPVATEQYGSVST |
| | | | | | NLQRGNRQAA |
| 25 | AAV9 Δ444- | AAV9 | C-terminal | Forms genome- | DGVGSSSGNWHCDSQWLGDRVITTSTRT |
| | 736 | Δ444-736 | truncation | protecting | WALPTYNNHLYKQISNSTSGGSSNDNAY |
| | 428insGS4 | 428ins | with | spherical capsids | FGYSTPWGYFDFNRFHCHFSPRDWQRLI |
| | | (G/S)4 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHSGGGSQSLDRLMNPLIDQ |
| | | | | | Y |
| 26 | AAV9 Δ593- | AAV9 | C-terminal | Forms genome- | DGVGSSSGNWHCDSQWLGDRVITTSTRT |
| | 736 | Δ593-736 | truncation | protecting | WALPTYNNHLYKQISNSTSGGSSNDNAY |
| | 428insGS4 | 428ins | with | spherical capsids | FGYSTPWGYFDFNRFHCHFSPRDWQRLI |
| | | (G/S)4 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHSGGGSQSLDRLMNPLIDQ |
| | | | | | YLYYLSKTINGSGQNQQTLKFSVAGPSN |
| | | | | | MAVQGRNYIPGPSYRQQRVSTTVTQNNN |
| | | | | | SEFAWPGASSWALNGRNSLMNPGPAMA |
| | | | | | SHKEGEDRFFPLSGSLIFGKQGTGRDNVD |
| | | | | | ADKVMITNEEEIKTTNPVATESYGQVATN |
| | | | | | HQSAQAQAQ |
| 27 | AAVDJ | AAVDJ | C-terminal | Forms genome- | GVGNSSGNWHCDSTWMGDRVITTSTRT |
| | Δ450-736 | Δ450-736 | truncation | protecting | WALPTYNNHLYKQISNSTSGGSSNDNAY |
| | 428insGS5 | 428ins | with | spherical capsids | FGYSTPWGYFDFNRFHCHFSPRDWQRLI |
| | | (G/S)5 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGSGGGSQSLDRLMNPLI |
| | | | | | DQYLYYLSR |
| 28 | AAVDJ | AAVDJ | C-terminal | Forms genome- | GVGNSSGNWHCDSTWMGDRVITTSTRT |
| | Δ594-736 | Δ594-736 | truncation | protecting | WALPTYNNHLYKQISNSTSGGSSNDNAY |
| | 428insGS5 | 428ins | with | spherical capsids | FGYSTPWGYFDFNRFHCHFSPRDWQRLI |
| | | (G/S)5 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGSGGGSQSLDRLMNPLI |
| | | | | | DQYLYYLSRTQTTGGTTNTQTLGFSQGG |
| | | | | | PNTMANQAKNWLPGPCYRQQRVSKTSA |
| | | | | | DNNNSEYSWTGATKYHLNGRDSLVNPGP |
| | | | | | AMASHKDDEEKFFPQSGVLIFGKQGSEKT |
| | | | | | NVDIEKVMITDEEEIRTTNPVATEQYGSV |
| | | | | | STNLQRGNRQAA |
| 29 | AAV9 Δ444- | AAV9 | C-terminal | Forms genome- | DGVGSSSGNWHCDSQWLGDRVITTSTRT |
| | 736 | Δ444-736 | truncation | protecting | WALPTYNNHLYKQISNSTSGGSSNDNAY |
| | 428insGS5 | 428ins | with | spherical capsids | FGYSTPWGYFDFNRFHCHFSPRDWQRLI |
| | | (G/S)5 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGSGGGSQSLDRLMNPLID |
| | | | | | QY |
| 30 | AAV9 Δ593- | AAV9 | C-terminal | Forms genome- | DGVGSSSGNWHCDSQWLGDRVITTSTRT |
| | 736 | Δ593-736 | truncation | protecting | WALPTYNNHLYKQISNSTSGGSSNDNAY |
| | 428insGS5 | 428ins | with | spherical capsids | FGYSTPWGYFDFNRFHCHFSPRDWQRLI |
| | | (G/S)5 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGSGGGSQSLDRLMNPLID |
| | | | | | QYLYYLSKTINGSGQNQQTLKFSVAGPSN |
| | | | | | MAVQGRNYIPGPSYRQQRVSTTVTQNNN |
| | | | | | SEFAWPGASSWALNGRNSLMNPGPAMA |
| | | | | | SHKEGEDRFFPLSGSLIFGKQGTGRDNVD |
| | | | | | ADKVMITNEEEIKTTNPVATESYGQVATN |
| | | | | | HQSAQAQAQ |
| 31 | AAVDJ | AAVDJ | C-terminal | Forms genome- | GVGNSSGNWHCDSTWMGDRVITTSTRT |
| | Δ450-736 | Δ450-736 | truncation | protecting | WALPTYNNHLYKQISNSTSGGSSNDNAY |
| | 428insGS6 | 428ins | with | spherical capsids | FGYSTPWGYFDFNRFHCHFSPRDWQRLI |
| | | (G/S)6 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGSQSLDRLMNP |
| | | | | | LIDQYLYYLSR |
| 32 | AAVDJ | AAVDJ | C-terminal | Forms genome- | GVGNSSGNWHCDSTWMGDRVITTSTRT |
| | Δ594-736 | Δ594-736 | truncation | protecting | WALPTYNNHLYKQISNSTSGGSSNDNAY |
| | 428insGS6 | 428ins | with | spherical capsids | FGYSTPWGYFDFNRFHCHFSPRDWQRLI |
| | | (G/S)6 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGSQSLDRLMNP |
| | | | | | LIDQYLYYLSRTQTTGGTTNTQTLGFSQG |
| | | | | | GPNTMANQAKNWLPGPCYRQQRVSKTS |
| | | | | | ADNNNSEYSWTGATKYHLNGRDSLVNP |
| | | | | | GPAMASHKDDEEKFFPQSGVLIFGKQGSE |
| | | | | | KTNVDIEKVMITDEEEIRTTNPVATEQYG |
| | | | | | SVSTNLQRGNRQAA |
| 33 | AAV9 Δ444- | AAV9 | C-terminal | Forms genome- | DGVGSSSGNWHCDSQWLGDRVITTSTRT |
| | 736 | Δ444-736 | truncation | protecting | WALPTYNNHLYKQISNSTSGGSSNDNAY |
| | 428insGS6 | 428ins | with | spherical capsids | FGYSTPWGYFDFNRFHCHFSPRDWQRLI |
| | | (G/S)6 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGSQSLDRLMNPLI |
| | | | | | DQY |
| 34 | AAV9 Δ593- | AAV9 | C-terminal | Forms genome- | DGVGSSSGNWHCDSQWLGDRVITTSTRT |
| | 736 | Δ593-736 | truncation | protecting | WALPTYNNHLYKQISNSTSGGSSNDNAY |
| | 428insGS6 | 428ins | with | spherical capsids | FGYSTPWGYFDFNRFHCHFSPRDWQRLI |
| | | (G/S)7 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGSQSLDRLMNPLI |
| | | | | | DQYLYYLSKTINGSGQNQQTLKFSVAGPS |
| | | | | | NMAVQGRNYIPGPSYRQQRVSTTVTQNN |
| | | | | | NSEFAWPGASSWALNGRNSLMNPGPAM |
| | | | | | ASHKEGEDRFFPLSGSLIFGKQGTGRDNV |
| | | | | | DADKVMITNEEEIKTTNPVATESYGQVAT |
| | | | | | NHQSAQAQAQ |
| 35 | AAVDJ | AAVDJ | C-terminal | Forms genome- | GVGNSSGNWHCDSTWMGDRVITTSTRT |
| | Δ450-736 | Δ450-736 | truncation | protecting | WALPTYNNHLYKQISNSTSGGSSNDNAY |
| | 428insGS7 | 428ins | with | spherical capsids | FGYSTPWGYFDFNRFHCHFSPRDWQRLI |
| | | (G/S)7 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGGSQSLDRLMN |
| | | | | | PLIDQYLYYLSR |
| 36 | AAVDJ | AAVDJ | C-terminal | Forms genome- | GVGNSSGNWHCDSTWMGDRVITTSTRT |
| | Δ594-736 | Δ594-736 | truncation | protecting | WALPTYNNHLYKQISNSTSGGSSNDNAY |
| | 428insGS7 | 428ins | with | spherical capsids | FGYSTPWGYFDFNRFHCHFSPRDWQRLI |
| | | (G/S)7 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGGSQSLDRLMN |
| | | | | | PLIDQYLYYLSRTQTTGGTTNTQTLGFSQ |
| | | | | | GGPNTMANQAKNWLPGPCYRQQRVSKT |
| | | | | | SADNNNSEYSWTGATKYHLNGRDSLVNP |
| | | | | | GPAMASHKDDEEKFFPQSGVLIFGKQGSE |
| | | | | | KTNVDIEKVMITDEEEIRTTNPVATEQYG |
| | | | | | SVSTNLQRGNRQAA |
| 37 | AAV9 Δ444- | AAV9 | C-terminal | Forms genome- | DGVGSSSGNWHCDSQWLGDRVITTSTRT |
| | 736 | Δ444-736 | truncation | protecting | WALPTYNNHLYKQISNSTSGGSSNDNAY |
| | 428insGS7 | 428ins | with | spherical capsids | FGYSTPWGYFDFNRFHCHFSPRDWQRLI |
| | | (G/S)7 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGGSQSLDRLMNP |
| | | | | | LIDQY |
| 38 | AAV9 Δ593- | AAV9 | C-terminal | Forms genome- | DGVGSSSGNWHCDSQWLGDRVITTSTRT |
| | 736 | Δ593-736 | truncation | protecting | WALPTYNNHLYKQISNSTSGGSSNDNAY |
| | 428insGS7 | 428ins | with | spherical capsids | FGYSTPWGYFDFNRFHCHFSPRDWQRLI |
| | | (G/S)7 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGGSQSLDRLMNP |
| | | | | | LIDQYLYYLSKTINGSGQNQQTLKFSVAG |
| | | | | | PSNMAVQGRNYIPGPSYRQQRVSTTVTQ |
| | | | | | NNNSEFAWPGASSWALNGRNSLMNPGP |
| | | | | | AMASHKEGEDRFFPLSGSLIFGKQGTGRD |
| | | | | | NVDADKVMITNEEEIKTTNPVATESYGQ |
| | | | | | VATNHQSAQAQAQ |
| 39 | AAVDJ | AAVDJ | C-terminal | Forms genome- | GVGNSSGNWHCDSTWMGDRVITTSTRT |
| | Δ450-736 | Δ450-736 | truncation | protecting | WALPTYNNHLYKQISNSTSGGSSNDNAY |
| | 428insGS8 | 428ins | with | spherical capsids | FGYSTPWGYFDFNRFHCHFSPRDWQRLI |
| | | (G/S)7 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGGGSQSLDRLM |
| | | | | | NPLIDQYLYYLSR |
| 40 | AAVDJ | AAVDJ | C-terminal | Forms genome- | GVGNSSGNWHCDSTWMGDRVITTSTRT |
| | Δ594-736 | Δ594-736 | truncation | protecting | WALPTYNNHLYKQISNSTSGGSSNDNAY |
| | 428insGS8 | 428ins | with | spherical capsids | FGYSTPWGYFDFNRFHCHFSPRDWQRLI |
| | | (G/S)7 | ″hinge″ | with | NNNWGFRPKRLSFKLFNIQVKEVTQNEG |
| | | | extensions | heterogeneous | TKTIANNLTSTIQVFTDSEYQLPYVLGSA |
| | | | | sizes. Infectious. | HQGCLPPFPADVFMIPQYGYLTLNNGSQ |
| | | | | | AVGRSSFYCLEYFPSQMLRTGNNFQFTYT |
| | | | | | FEDVPFHSSYAHGGGSGGGGSQSLDRLM |
| | | | | | NPLIDQYLYYLSRTQTTGGTTNTQTLGFS |
| | | | | | QGGPNTMANQAKNWLPGPCYRQQRVSK |
| | | | | | TSADNNNSEYSWTGATKYHLNGRDSLV |
| | | | | | NPGPAMASHKDDEEKFFPQSGVLIFGKQ |
| | | | | | GSEKTNVDIEKVMITDEEEIRTTNPVATE |
| | | | | | QYGSVSTNLQRGNRQAA |
| 41 | AAV9 Δ444- | AAV9 | C-terminal | Forms genome- | DGVGSSSGNWHCDSQWLGDRVITTSTRT |
| | 736 | Δ444-736 | truncation | protecting | WALPTYNNHLYKQISNSTSGGSSNDNAY |
| | 428insGS8 | 428ins | with | spherical capsids | FGYSTPWGYFDFNRFHCHFSPRDWQRLI |
| | | (G/S)7 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGGGSQSLDRLMN |
| | | | | | PLIDQY |
| 42 | AAV9 Δ593- | AAV9 | C-terminal | Forms genome- | DGVGSSSGNWHCDSQWLGDRVITTSTRT |
| | 736 | Δ593-736 | truncation | protecting | WALPTYNNHLYKQISNSTSGGSSNDNAY |
| | 428insGS8 | 428ins | with | spherical capsids | FGYSTPWGYFDFNRFHCHFSPRDWQRLI |
| | | (G/S)7 | ″hinge″ | with | NNNWGFRPKRLNFKLFNIQVKEVTDNNG |
| | | | extensions | heterogeneous | VKTIANNLTSTVQVFTDSDYQLPYVLGSA |
| | | | | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSYAHGGGSGGGGSQSLDRLMN |
| | | | | | PLIDQYLYYLSKTINGSGQNQQTLKFSVA |
| | | | | | GPSNMAVQGRNYIPGPSYRQQRVSTTVT |
| | | | | | QNNNSEFAWPGASSWALNGRNSLMNPG |
| | | | | | PAMASHKEGEDRFFPLSGSLIFGKQGTGR |
| | | | | | DNVDADKVMITNEEEIKTTNPVATESYG |
| | | | | | QVATNHQSAQAQAQ |
| 43 | Δ444-736 | N/A | C-terminal | Forms genome- | DGVGSSSGNWHCDSQWLGDRVITTSTRT |
| | YAH426- | | truncated | protecting | WALPTYNNHLYKQISNSTSGGSSNDNAY |
| | 428GS8 | | variant | spherical capsids | FGYSTPWGYFDFNRFHCHFSPRDWQRLI |
| | | | with hinge | with | NNNWGFRPKRLNFKLFNIQVKEVTDNNG |
| | | | modificati | heterogeneous | VKTIANNLTSTVQVFTDSDYQLPYVLGSA |
| | | | ons | sizes. Infectious. | HEGCLPPFPADVFMIPQYGYLTLNDGSQA |
| | | | | | VGRSSFYCLEYFPSQMLRTGNNFQFSYEF |
| | | | | | ENVPFHSSGGGSG |
| 44 | Δ417-640- | N/A | Domain | Forms genome- | DGVGSSSGNWHCDSQWLGDRVITTSTRT |
| | GGS-430- | | swap | protecting capsids | WALPTYNNHLYKQISNSTSGGSSNDNAY |
| | 640 | | (adding | | FGYSTPWGYFDFNRFHCHFSPRDWQRLI |
| | | | back part | | NNNWGFRPKRLNFKLFNIQVKEVTDNNG |
| | | | of spike | | VKTIANNLTSTVQVFTDSDYQLPYVLGSA |
| | | | block after | | HEGCLPPFPADVFMIPQYGYLTLNDGSQA |
| | | | a spike- | | VGRSSFYCLEYFPSQMLRTGNNFQFSYEK |
| | | | deleted | | HPPPQILIKNTPVPADPPTAFNKDKLNSFIT |
| | | | variant) | | QYSTGQVSVEIEWELQKENSKRWNPEIQ |
| | | | | | YTSNYYKSNNVEFAVNTEGVYSEPRPIGT |
| | | | | | RYLTRNLGGSDRLMNPLIDQYLYYLSKTI |
| | | | | | NGSGQNQQTLKFSVAGPSNMAVQGRNYI |
| | | | | | PGPSYRQQRVSTTVTQNNNSEFAWPGAS |
| | | | | | SWALNGRNSLMNPGPAMASHKEGEDRF |
| | | | | | FPLSGSLIFGKQGTGRDNVDADKVMITNE |
| | | | | | EEIKTTNPVATESYGQVATNHQSAQAQA |
| | | | | | QTGWVQNQGILPGMVWQDRDVYLQGPI |
| | | | | | WAKIPHTDGNFHPSPLMGGFGM |
| 45 | AAV9 Δ417- | N/A | Domain | Forms genome- | DGVGSSSGNWHCDSQWLGDRVITTSTRT |
| | 640-G7-430- | | swap | protecting capsids | WALPTYNNHLYKQISNSTSGGSSNDNAY |
| | 640 | | (adding | | FGYSTPWGYFDFNRFHCHFSPRDWQRLI |
| | | | back part | | NNNWGFRPKRLNFKLFNIQVKEVTDNNG |
| | | | of spike | | VKTIANNLTSTVQVFTDSDYQLPYVLGSA |
| | | | block after | | HEGCLPPFPADVFMIPQYGYLTLNDGSQA |
| | | | a spike- | | VGRSSFYCLEYFPSQMLRTGNNFQFSYEK |
| | | | deleted | | HPPPQILIKNTPVPADPPTAFNKDKLNSFIT |
| | | | variant) | | QYSTGQVSVEIEWELQKENSKRWNPEIQ |
| | | | | | YTSNYYKSNNVEFAVNTEGVYSEPRPIGT |
| | | | | | RYLTRNLGGSGGGSDRLMNPLIDQYLYY |
| | | | | | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVQNQGILPGMVWQDRDVY |
| | | | | | LQGPIWAKIPHTDGNFHPSPLMGGFGM |
| 46 | AAV9 Δ445- | N/A | Combined | Forms genome- | DGVGSSSGNWHCDSQWLGDRVITTSTRT |
| | 610 GS9 | | spike | protecting capsids | WALPTYNNHLYKQISNSTSGGSSNDNAY |
| | 428insGS6 | | truncation | and transduces | FGYSTPWGYFDFNRFHCHFSPRDWQRLI |
| | | | and hinge | oversized | NNNWGFRPKRLNFKLFNIQVKEVTDNNG |
| | | | extension | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHGGSGGGSQSLDRLMNPLI |
| | | | | | DQYLGGGGSGGGSDVYLQGPIWAKIPHT |
| | | | | | DGNFHPSPLMGGFGMKHPPPQILIKNTPV |
| | | | | | PADPPTAFNKDKLNSFITQYSTGQVSVEIE |
| | | | | | WELQKENSKRWNPEIQYTSNYYKSNNVE |
| | | | | | FAVNTEGVYSEPRPIGTRYLTRNL |
| 47 | AAV-DJ | N/A | Combined | Forms genome- | GVGNSSGNWHCDSTWMGDRVITTSTRT |
| | Δ445-611 | | spike | protecting capsids | WALPTYNNHLYKQISNSTSGGSSNDNAY |
| | GS7 | | truncation | and transduces | FGYSTPWGYFDFNRFHCHFSPRDWQRLI |
| | 428insGS6 | | and hinge | oversized | NNNWGFRPKRLSFKLFNIQVKEVTQNEG |
| | | | extension | genomes to cells | TKTIANNLTSTIQVFTDSEYQLPYVLGSA |
| | | | | | HQGCLPPFPADVFMIPQYGYLTLNNGSQ |
| | | | | | AVGRSSFYCLEYFPSQMLRTGNNFQFTYT |
| | | | | | FEDVPFHSSYAHGGSGGGSQSLDRLMNP |
| | | | | | LIDQYLGGSGGGSDVYLQGPIWAKIPHTD |
| | | | | | GHFHPSPLMGGFGLKHPPPQILIKNTPVPA |
| | | | | | DPPTTFNQSKLNSFITQYSTGQVSVEIEWE |
| | | | | | LQKENSKRWNPEIQYTSNYYKSTSVDFA |
| | | | | | VNTEGVYSEPRPIGTRYLTRNL |
| 48 | AAV9 | Triple | Combined | Forms genome- | DGVGSSSGNWHCDSQWLGDRVITTSTRT |
| | Δ445- | trim 0c | spike | protecting capsids | WALPTYNNHLYKQISNSTSGGSSNDNAY |
| | 640GS15 | | truncation | and transduces | FGYSTPWGYFDFNRFHCHFSPRDWQRLI |
| | Δ652-672GS | | and sealer | oversized | NNNWGFRPKRLNFKLFNIQVKEVTDNNG |
| | Δ706-736 | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGGSGGGGSKHPPPQILIKNGSQYS |
| | | | | | TGQVSVEIEWELQKENSKRWNPEIQYTSN |
| | | | | | Y |
| 49 | AAV9 Δ445- | Triple | Combined | Forms genome- | DGVGSSSGNWHCDSQWLGDRVITTSTRT |
| | 640GS15 | trim 1b | spike | protecting capsids | WALPTYNNHLYKQISNSTSGGSSNDNAY |
| | Δ659-666GS | | truncation | and transduces | FGYSTPWGYFDFNRFHCHFSPRDWQRLI |
| | Δ706-736 | | and sealer | oversized | NNNWGFRPKRLNFKLFNIQVKEVTDNNG |
| | | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGGSGGGGSKHPPPQILIKNTPVPA |
| | | | | | DPGSLNSFITQYSTGQVSVEIEWELQKEN |
| | | | | | SKRWNPEIQYTSNY |
| 50 | AAV9 Δ445- | N/A | Combined | Forms genome- | DGVGSSSGNWHCDSQWLGDRVITTSTRT |
| | 603 GS7 | | spike | protecting capsids | WALPTYNNHLYKQISNSTSGGSSNDNAY |
| | Δ706-736 | | truncation | and transduces | FGYSTPWGYFDFNRFHCHFSPRDWQRLI |
| | | | and sealer | oversized | NNNWGFRPKRLNFKLFNIQVKEVTDNNG |
| | | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGSGMVWQDRDVYLQGPIWAKIP |
| | | | | | HTDGNFHPSPLMGGFGMKHPPPQILIKNT |
| | | | | | PVPADPPTAFNKDKLNSFITQYSTGQVSV |
| | | | | | EIEWELQKENSKRWNPEIQYTSNY |
| 51 | AAV9 Δ445- | N/A | Combined | Forms genome- | DGVGSSSGNWHCDSQWLGDRVITTSTRT |
| | 610GSS9 | | spike | protecting capsids | WALPTYNNHLYKQISNSTSGGSSNDNAY |
| | Δ706-736 | | truncation | and transduces | FGYSTPWGYFDFNRFHCHFSPRDWQRLI |
| | | | and sealer | oversized | NNNWGFRPKRLNFKLFNIQVKEVTDNNG |
| | | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHGGSGGGSQSLDRLMNPLI |
| | | | | | DQYLGGGGSGGGSDVYLQGPIWAKIPHT |
| | | | | | DGNFHPSPLMGGFGMKHPPPQILIKNTPV |
| | | | | | PADPPTAFNKDKLNSFITQYSTGQVSVEIE |
| | | | | | WELQKENSKRWNPEIQYTSNY |
| 52 | AAV9 Δ445- | N/A | Combined | Forms genome- | DGVGSSSGNWHCDSQWLGDRVITTSTRT |
| | Δ659-666 GS | | truncation | and transduces | FGYSTPWGYFDFNRFHCHFSPRDWQRLI |
| | Δ704-727G | | and sealer | oversized | NNNWGFRPKRLNFKLFNIQVKEVTDNNG |
| | | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GSGGGSGGSKHPPPQILIKNTPVPADPGSL |
| | | | | | NSFITQYSTGQVSVEIEWELQKENSKRWN |
| | | | | | PEIQYTSGGTRYLTRNL |
| 53 | AAV9 Δ445- | N/A | Combined | Forms genome- | DGVGSSSGNWHCDSQWLGDRVITTSTRT |
| | 610 G9 | | spike | protecting capsids | WALPTYNNHLYKQISNSTSGGSSNDNAY |
| | Δ659-666 GS | | truncation | and transduces | FGYSTPWGYFDFNRFHCHFSPRDWQRLI |
| | Δ704-727G | | and sealer | oversized | NNNWGFRPKRLNFKLFNIQVKEVTDNNG |
| | | | truncation | genomes to cells | VKTIANNLTSTVQVFTDSDYQLPYVLGSA |
| | | | | | HEGCLPPFPADVFMIPQYGYLTLNDGSQA |
| | | | | | VGRSSFYCLEYFPSQMLRTGNNFQFSYEF |
| | | | | | ENVPFHSSYAHSQSLDRLMNPLIDQYLGG |
| | | | | | GGSGGGSDVYLQGPIWAKIPHTDGNFHPS |
| | | | | | PLMGGFGMKHPPPQILIKNTPVPADPGSL |
| | | | | | NSFITQYSTGQVSVEIEWELQKENSKRWN |
| | | | | | PEIQYTSGGTRYLTRNL |
| 54 | TripleTrim0 | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS12 | | in both | | FGYSTPWGYFDFNRFHCHFSPRDWQRLI |
| | Δ652- | | spike | | NNNWGFRPKRLNFKLFNIQVKEVTDNNG |
| | 671GGS | | block and | | VKTIANNLTSTVQVFTDSDYQLPYVLGSA |
| | Δ692-736) | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGSGGS |
| | | | | | GGSGGSKHPPPQILIKNGGSTQYSTGQVS |
| | | | | | VEIEWELQKEN |
| 55 | TripleTrim0 | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS12 | | in both | | FGYSTPWGYFDFNRFHCHFSPRDWQRLI |
| | Δ652- | | spike | | NNNWGFRPKRLNFKLFNIQVKEVTDNNG |
| | 671GGS | | block and | | VKTIANNLTSTVQVFTDSDYQLPYVLGSA |
| | Δ692-736) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGSGGSGGSGGSKHPPPQILIKNGGSTQY |
| | | | | | STGQVSVEIEWELQKEN |
| 56 | TripleTrim0a | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS12 | | in both | | FGYSTPWGYFDFNRFHCHFSPRDWQRLI |
| | Δ652-672GS | | spike | | NNNWGFRPKRLNFKLFNIQVKEVTDNNG |
| | Δ692-736) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGSGGS |
| | | | | | GGSGGSKHPPPQILIKNGSQYSTGQVSVEI |
| | | | | | EWELQKEN |
| 57 | TripleTrim0a | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS12 | | in both | | FGYSTPWGYFDFNRFHCHFSPRDWQRLI |
| | Δ652-672GS | | spike | | NNNWGFRPKRLNFKLFNIQVKEVTDNNG |
| | Δ692-736) | | block and | | VKTIANNLTSTVQVFTDSDYQLPYVLGSA |
| | with | | sealer | | HEGCLPPFPADVFMIPQYGYLTLNDGSQA |
| | extended | | block | | VGRSSFYCLEYFPSQMLRTGNNFQFSYEF |
| | hinge | | | | ENVPFHSSYAHSGGSGGSLCNTRNMNPLI |
| | | | | | GGSGGSGGSGGSKHPPPQILIKNGSQYST |
| | | | | | GQVSVEIEWELQKEN |
| 58 | TripleTrim0b | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ652-672GS | | spike | | NNNWGFRPKRLNFKLFNIQVKEVTDNNG |
| | Δ692-736) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGGGSG |
| | | | | | GGGSGGGGSKHPPPQILIKNGSQYSTGQV |
| | | | | | SVEIEWELQKEN |
| 59 | TripleTrim0b | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ652-672GS | | spike | | NNNWGFRPKRLNFKLFNIQVKEVTDNNG |
| | Δ692-736) | | block and | | VKTIANNLTSTVQVFTDSDYQLPYVLGSA |
| | with | | sealer | | HEGCLPPFPADVFMIPQYGYLTLNDGSQA |
| | extended | | block | | VGRSSFYCLEYFPSQMLRTGNNFQFSYEF |
| | hinge | | | | ENVPFHSSYAHSGGSGGSLCNTRNMNPLI |
| | | | | | GGGGSGGGGSGGGGSKHPPPQILIKNGSQ |
| | | | | | YSTGQVSVEIEWELQKEN |
| 60 | TripleTrim0c | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ652-672GS | | spike | | NNNWGFRPKRLNFKLFNIQVKEVTDNNG |
| | Δ706-736) | | block and | | VKTIANNLTSTVQVFTDSDYQLPYVLGSA |
| | with | | sealer | | HEGCLPPFPADVFMIPQYGYLTLNDGSQA |
| | extended | | block | | VGRSSFYCLEYFPSQMLRTGNNFQFSYEF |
| | hinge | | | | ENVPFHSSYAHSGGSGGSLCNTRNMNPLI |
| | | | | | GGGGSGGGGSGGGGSKHPPPQILIKNGSQ |
| | | | | | YSTGQVSVEIEWELQKENSKRWNPEIQYT |
| | | | | | SNY |
| 61 | TripleTrim0d | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ652-672GS | | spike | | NNNWGFRPKRLNFKLFNIQVKEVTDNNG |
| | Δ704-727G) | | block and | | VKTIANNLTSTVQVFTDSDYQLPYVLGSA |
| | with | | sealer | | HEGCLPPFPADVFMIPQYGYLTLNDGSQA |
| | extended | | block | | VGRSSFYCLEYFPSQMLRTGNNFQFSYEF |
| | hinge | | | | ENVPFHSSYAHSGGSGGSLCNTRNMNPLI |
| | | | | | GGGGSGGGGSGGGGSKHPPPQILIKNGSQ |
| | | | | | YSTGQVSVEIEWELQKENSKRWNPEIQYT |
| | | | | | SGGTRYLTRNL |
| 62 | TripleTrim1 | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS12 | | in both | | FGYSTPWGYFDFNRFHCHFSPRDWQRLI |
| | Δ658-667GS | | spike | | NNNWGFRPKRLNFKLFNIQVKEVTDNNG |
| | Δ693-736) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIDQYLGG |
| | | | | | GSGGGSGGGSKHPPPQILIKNTPVPADGS |
| | | | | | NSFITQYSTGQVSVEIEWELQKENS |
| 63 | TripleTrim1 | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS12 | | spike | | NNNWGFRPKRLNFKLFNIQVKEVTDNNG |
| | Δ658-667GS | | block and | | VKTIANNLTSTVQVFTDSDYQLPYVLGSA |
| | Δ693-736) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSSGGSGGSLCNTRNQSLD |
| | hinge | | | | RLMNPLIDQYLGGGSGGGSGGGSKHPPP |
| | | | | | QILIKNTPVPADGSNSFITQYSTGQVSVEIE |
| | | | | | WELQKENS |
| 64 | TripleTrim1a | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ659-666GS | | spike | | NNNWGFRPKRLNFKLFNIQVKEVTDNNG |
| | Δ693-736) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGGGSG |
| | | | | | GGGSGGGGSKHPPPQILIKNTPVPADPGS |
| | | | | | LNSFITQYSTGQVSVEIEWELQKENS |
| 65 | TripleTrim1a | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ693-736) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGGGSGGGGSGGGGSKHPPPQILIKNTPV |
| | | | | | PADPGSLNSFITQYSTGQVSVEIEWELQK |
| | | | | | ENS |
| 66 | TripleTrim1b | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ706-736) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGGGSGGGGSGGGGSKHPPPQILIKNTPV |
| | | | | | PADPGSLNSFITQYSTGQVSVEIEWELQK |
| | | | | | ENSKRWNPEIQYTSNY |
| 67 | TripleTrim1e | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ704-727G) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGGGSGGGGSGGGGSKHPPPQILIKNTPV |
| | | | | | PADPGSLNSFITQYSTGQVSVEIEWELQK |
| | | | | | ENSKRWNPEIQYTSGGTRYLTRNL |
| 68 | TripleTrim2 | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ659-666GS | | spike | | NNNWGFRPKRLNFKLFNIQVKEVTDNNG |
| | Δ704-736) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGSGGS |
| | | | | | GGSGGSKHPPPQILIKNTPVPADPGSLNSF |
| | | | | | ITQYSTGQVSVEIEWELQKENSKRWNPEI |
| | | | | | QYTS |
| 69 | TripleTrim2 | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ704-736) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGSGGSGGSGGSKHPPPQILIKNTPVPADP |
| | | | | | GSLNSFITQYSTGQVSVEIEWELQKENSK |
| | | | | | RWNPEIQYTS |
| 70 | TripleTrim3 | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ659-666GS | | spike | | NNNWGFRPKRLNFKLFNIQVKEVTDNNG |
| | Δ704-727) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGSGGS |
| | | | | | GGSGGSKHPPPQILIKNTPVPADPGSLNSF |
| | | | | | ITQYSTGQVSVEIEWELQKENSKRWNPEI |
| | | | | | QYTSGTRYLTRNL |
| 71 | TripleTrim3 | N/A | Combined | Forms genome- | DGVGSSSGNWHCDSQWLGDRVITTSTRT |
| | (GS6LCNTRN | | truncations | protecting capsids | WALPTYNNHLYKQISNSTSGGSSNDNAY |
| | Δ445- | | in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ704-727) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGSGGSGGSGGSKHPPPQILIKNTPVPADP |
| | | | | | GSLNSFITQYSTGQVSVEIEWELQKENSK |
| | | | | | RWNPEIQYTSGTRYLTRNL |
| 72 | TripleTrim3a | N/A | Combined | Forms genome- | DGVGSSSGNWHCDSQWLGDRVITTSTRT |
| | (Δ445- | | truncations | protecting capsids | WALPTYNNHLYKQISNSTSGGSSNDNAY |
| | 640GS15 | | in both | | FGYSTPWGYFDFNRFHCHFSPRDWQRLI |
| | Δ659-666GS | | spike | | NNNWGFRPKRLNFKLFNIQVKEVTDNNG |
| | Δ704-727G) | | block and | | VKTIANNLTSTVQVFTDSDYQLPYVLGSA |
| | | | sealer | | HEGCLPPFPADVFMIPQYGYLTLNDGSQA |
| | | | block | | VGRSSFYCLEYFPSQMLRTGNNFQFSYEF |
| | | | | | ENVPFHSSYAHSQSLDRLMNPLIGGSGGS |
| | | | | | GGSGGSKHPPPQILIKNTPVPADPGSLNSF |
| | | | | | ITQYSTGQVSVEIEWELQKENSKRWNPEI |
| | | | | | QYTSGGTRYLTRNL |
| 73 | TripleTrim3a | N/A | Combined | Forms genome- | DGVGSSSGNWHCDSQWLGDRVITTSTRT |
| | (GS6LCNTRN | | truncation | protecting capsids | WALPTYNNHLYKQISNSTSGGSSNDNAY |
| | Δ445- | | s in both | | FGYSTPWGYFDFNRFHCHFSPRDWQRLI |
| | 640GS15 | | spike | | NNNWGFRPKRLNFKLFNIQVKEVTDNNG |
| | Δ659-666GS | | block and | | VKTIANNLTSTVQVFTDSDYQLPYVLGSA |
| | Δ704-727) | | sealer | | HEGCLPPFPADVFMIPQYGYLTLNDGSQA |
| | with | | block | | VGRSSFYCLEYFPSQMLRTGNNFQFSYEF |
| | extended | | | | ENVPFHSSYAHSGGSGGSLCNTRNMNPLI |
| | hinge | | | | GGSGGSGGSGGSKHPPPQILIKNTPVPADP |
| | | | | | GSLNSFITQYSTGQVSVEIEWELQKENSK |
| | | | | | RWNPEIQYTSGGTRYLTRNL |

The starting amino acid residue of the sequences in the above table is D219 for AAV9 and G220 for AAV-DJ.

### Example 2

### Structure-Guided Trimming of AAV Capsid Proteins Yields Size-Expanded Particles

Structural Analysis and Modeling Reveal Two Groups of Inter-Subunit Interactions that Cement AAV Capsid's Curvature

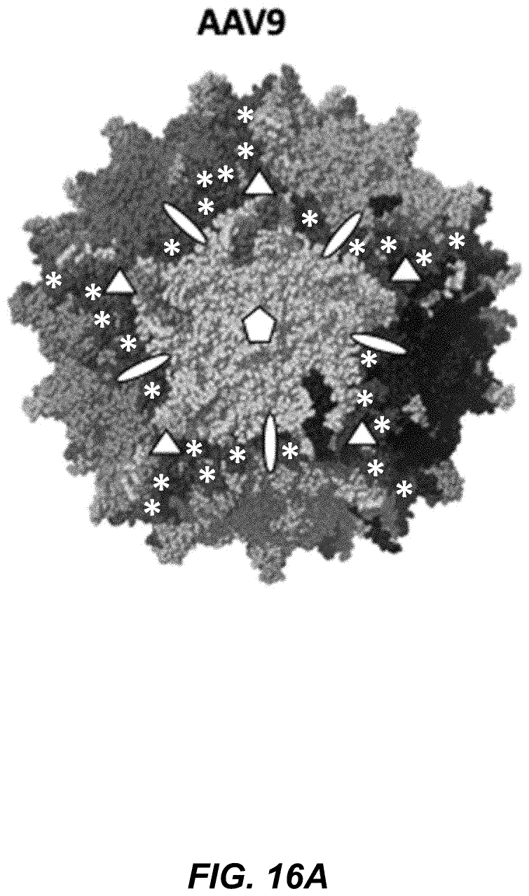

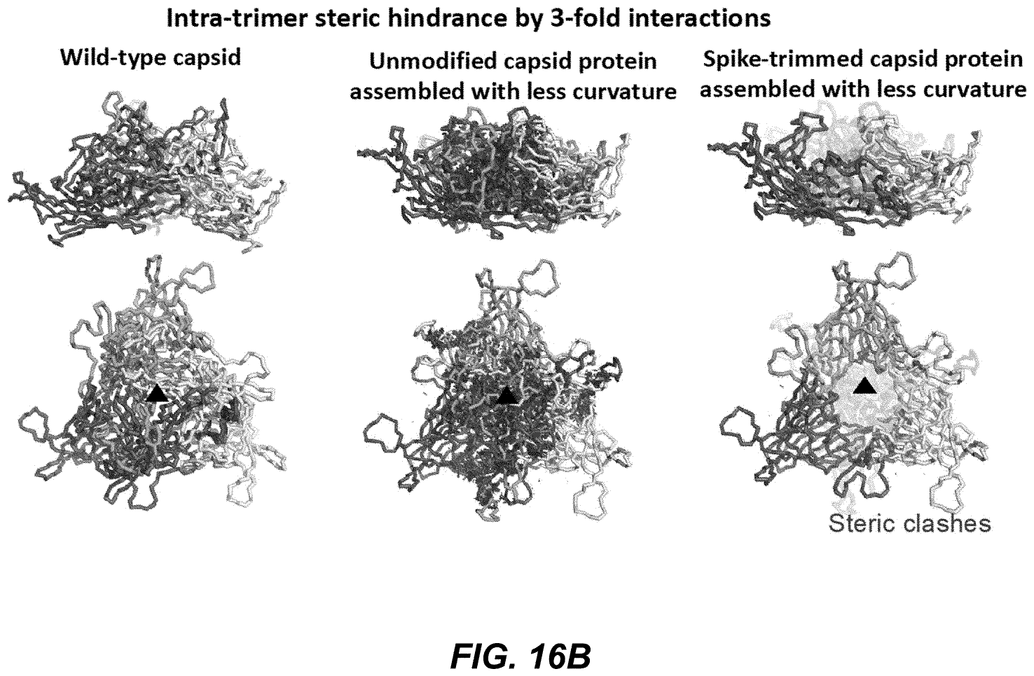

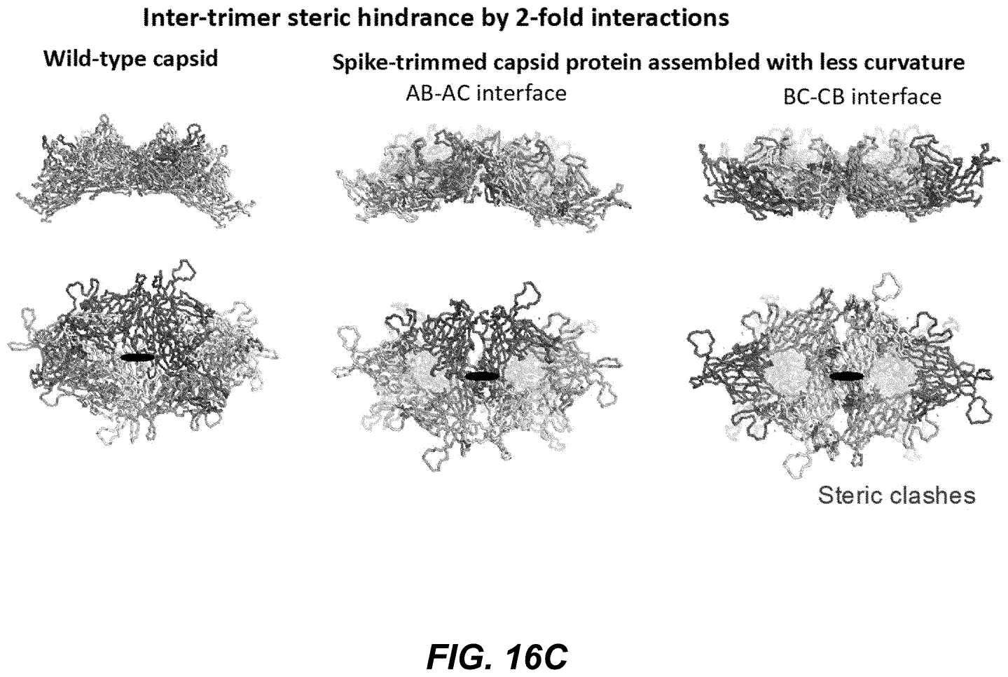

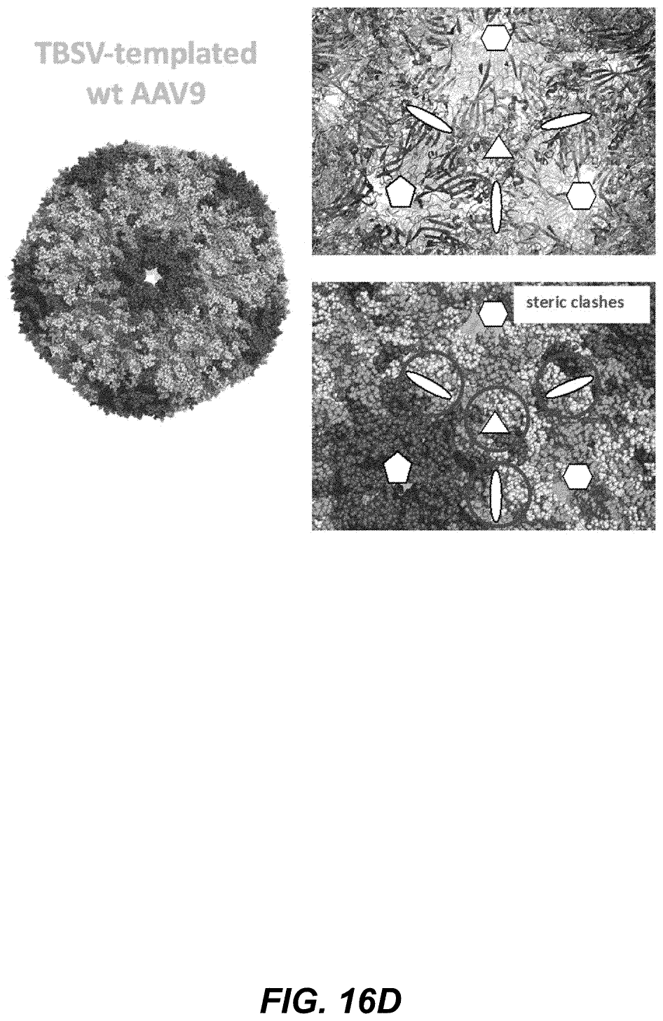

Increasing the diameter of capsids requires reducing their surface curvature. Structural analysis revealed that the curvature of an AAV capsid is mainly determined by the angle between relatively flat pentamers (FIG. 16A). These inter-pentamer angles are cemented by two groups of interactions, the interactions around the 3-fold axis at the center of a trimer, and the interactions around the 2-fold axis between two neighboring trimers (FIG. 16A-FIG. 16C).

In addition to the structural analysis, models of the architecture of a hallucinated AAV capsid with a larger radius of curvature were built to find out regions within the capsid protein sequence that prevents AAV from bending to a flatter shape. To get the relative angles between jelly rolls, TBSV, a single jelly-roll capsid that is made of 180 subunits and has a smaller surface curvature, was used as a template to align the AAV's jelly-roll region into it. To account for the size difference between individual subunits, the whole capsid was evenly expanded by ˜1.1 fold, i.e. moving each subunit farther from the center by 0.1 fold of its original distance. The modeling confirmed that two groups of interactions (i.e. interactions around the 3-fold spike and interactions around the 2-fold axis) can contribute to steric hindrance against AAV capsids from bending to a smaller curvature (FIG. 16B-FIG. 16D).

Loop Trimming Around the 3-Fold Axis Results in Size-Expanded Particles in the Cell Pellet and Capsid Protein/Genome Released to Media of Producer Cells

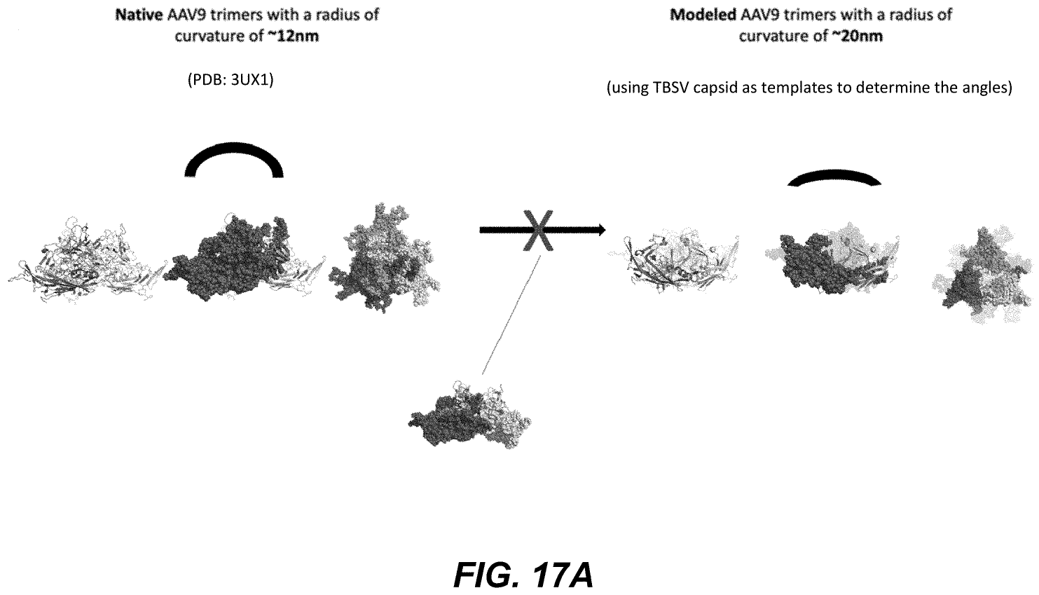

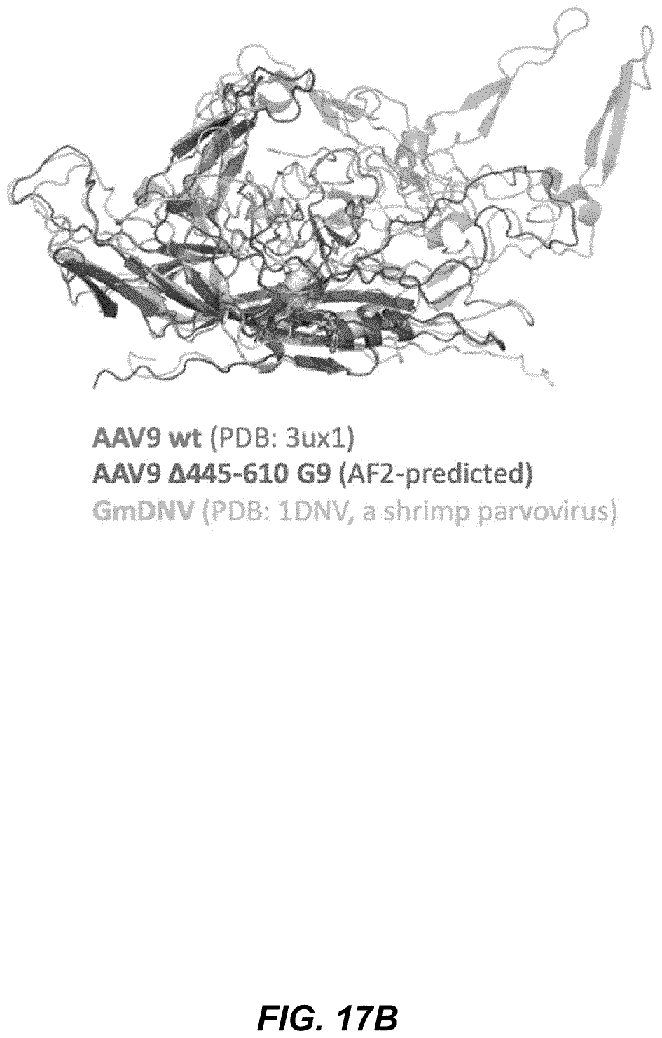

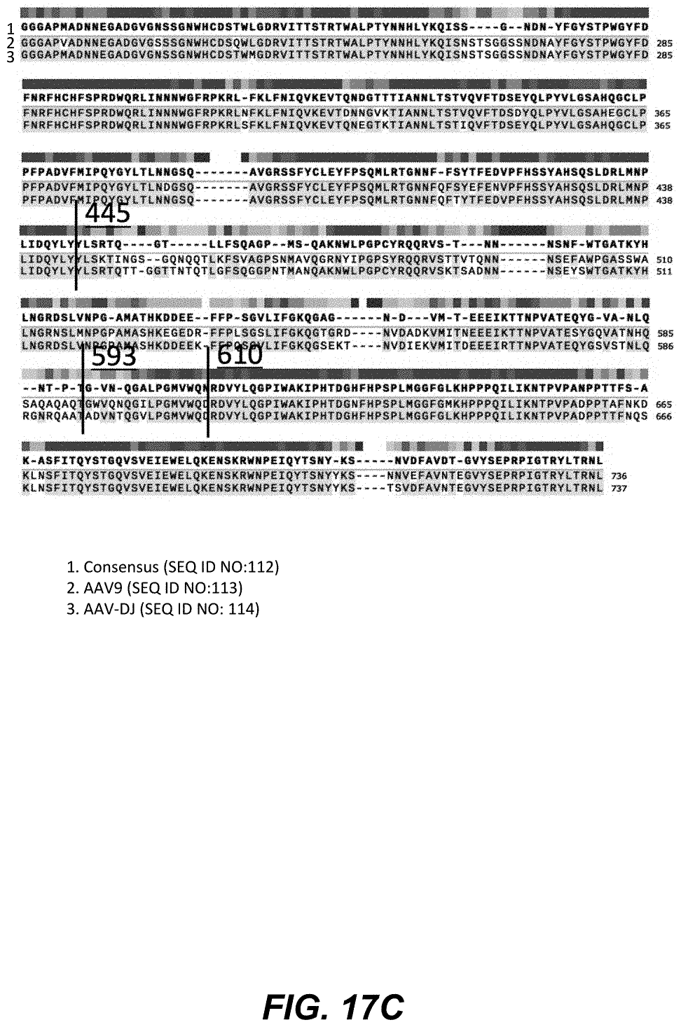

As analyzed above, the bulky, interlacing 3-fold interaction formed around the spikes (residues 418-640 in AAV9 VP1) can rigidify the curvature of AAV trimers, contributing to cementing the curvature of the capsid (FIG. 17A).

Without being bound by any particular theory, the spike region (residues 418-640 in AAV9 VP1) can tolerate large deletions. Firstly, such a bulky 3-fold spike region is not evolutionarily conserved. The spike region, particularly the long loop between beta strands G and H of the jelly-roll fold, is not present in single-jelly-roll capsids with larger sizes, such as capsids of TBSV, a 32-nm icosahedral capsid with the same single-jelly-roll protein fold, or GmDNV, a parvoviral relative of AAV that can package a ˜6.3 kb genome. Particularly, the VR-IV region of AAV (residues 445-463 in AAV9 VP1 indices), as well as the continuous region between VR-V-VR-VIII (residues 486-596 in AAV9 VP1 indices), showed drastically distinct structures compared to the corresponding regions in protopavovirus, implying that structures formed by this region are less conserved in Parvovirinae and can have evolved independently in the different sub-families after the appearance of the last common ancestor of Parvovirinae (FIG. 17B). Secondly, when the structure of unassembled monomeric AAV9 capsid protein was computationally predicted with Alphafold2, the resulting models suggested part of the spike region (residues 429-608) can fold into a domain separate from the core of the capsid protein in the pre-assembly state (FIG. 1A-FIG. 1C). Thirdly, amino acid residues in this region are much less conserved compared to the rest of the parts of the structured regions, as revealed by a sequence alignment done with capsid sequences of ˜100 serotypes (FIG. 17C). Fourthly, the region is the most tolerable to single-amino-acid mutations across the structured region.

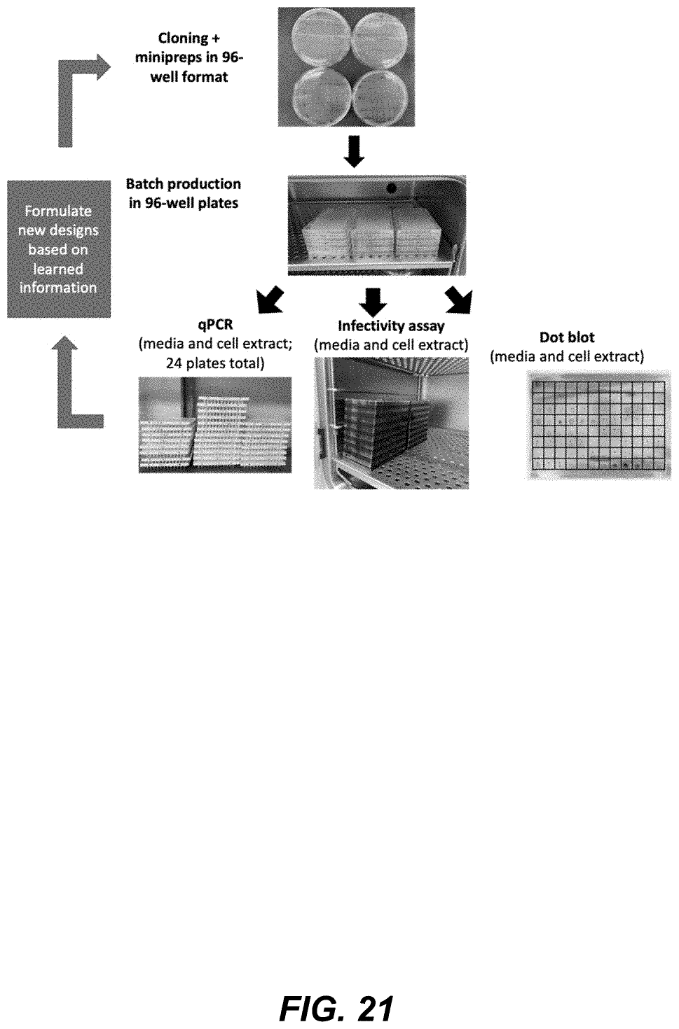

Based on the sequence alignment and structural predictions, capsid variants with large chunks of residues in the spike region trimmed and inserted flexible linkers of various lengths in replace were designed to maximally retain the native interactions. With iterative screening in 96-well format (FIG. 21), a few spike-truncated designs that assembled into capsids with enhanced yields of genome-protection titers when packaging an oversized genome were identified. Interestingly, the particle size of such variants is mostly around 30-50 nm, close to the expected size of a T=3 capsid.

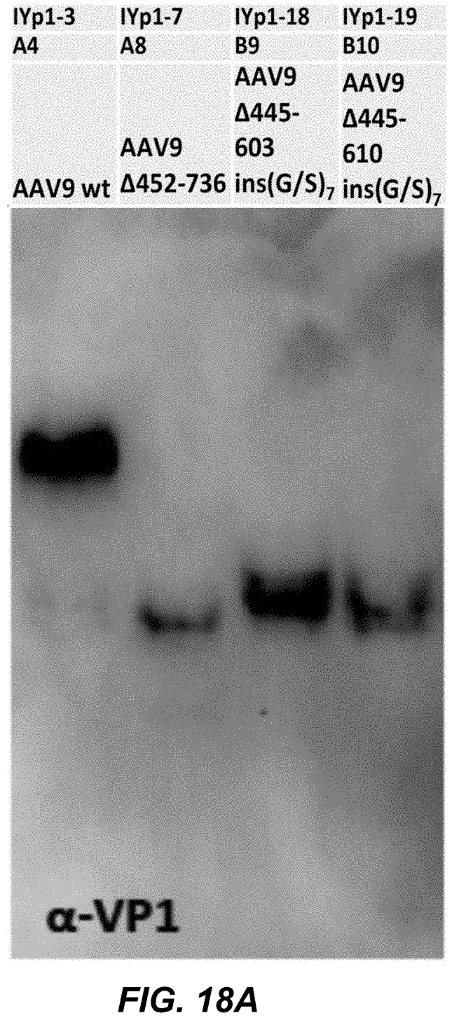

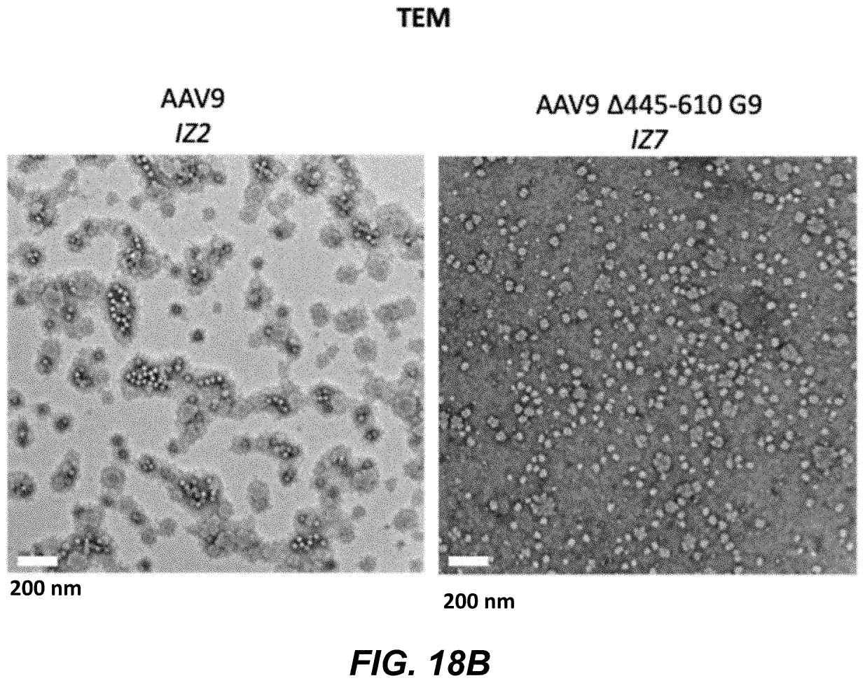

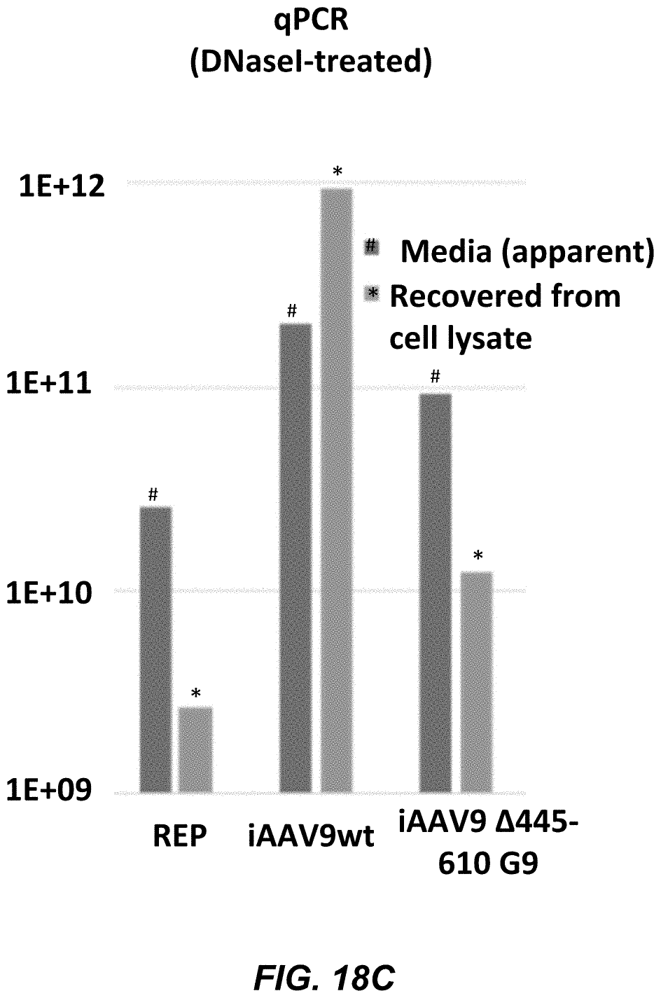

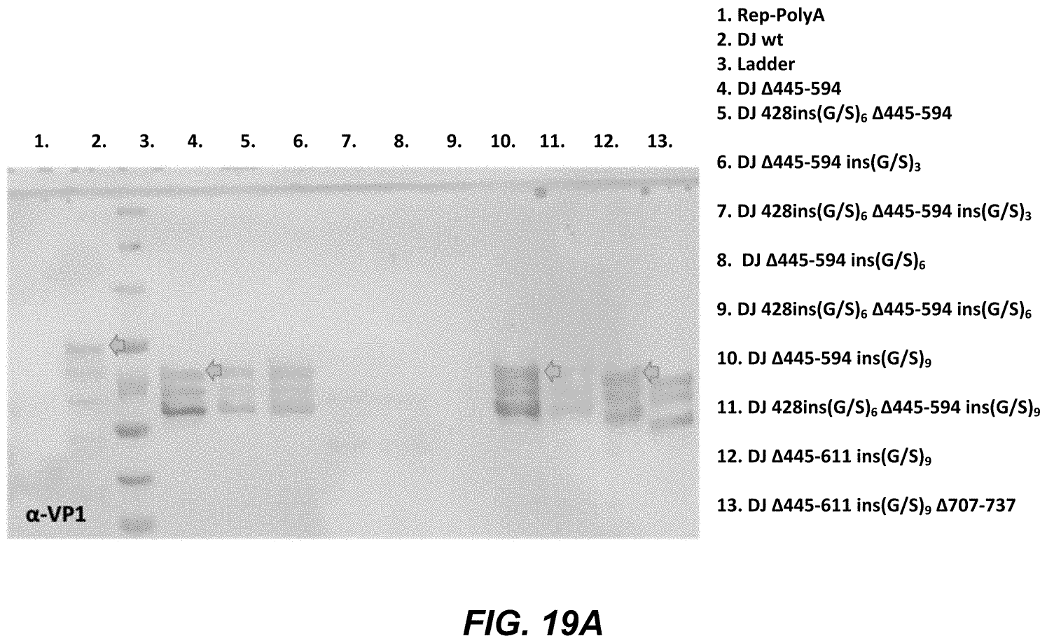

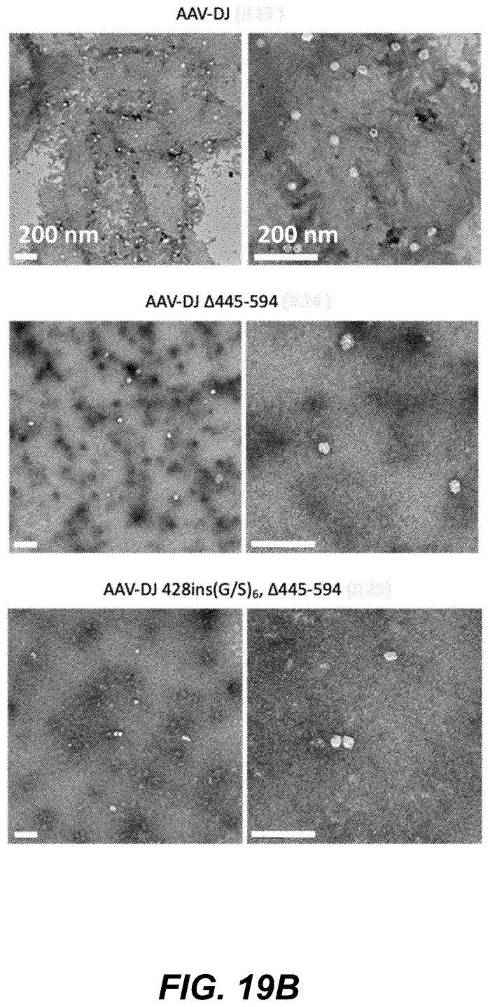

The first group of designs identified was AAV9 Δ445-610-like variants (or the counterpart truncation variants with other AAV serotypes, such as AAV-DJ Δ445-611-like variants). It made capsid proteins in the media (FIG. 18A) and the cell pellet (FIG. 19A), and the assembly product looked like 40 nm capsids when purified with a precipitation-based method (FIG. 18B). The capsids also showed some protection against DNase I treatment (FIG. 18C).

When the trimmed sequence was added back, a variant with a shorter deletion, AAV9 Δ445-593-like variants (or the counterpart truncation variants with other AAV serotypes, such as AAV-DJ Δ445-594-like variants), gave a higher yield of capsid proteins in the cell lysate (FIG. 19A) and yielded size-expanded capsids (FIG. 19B).

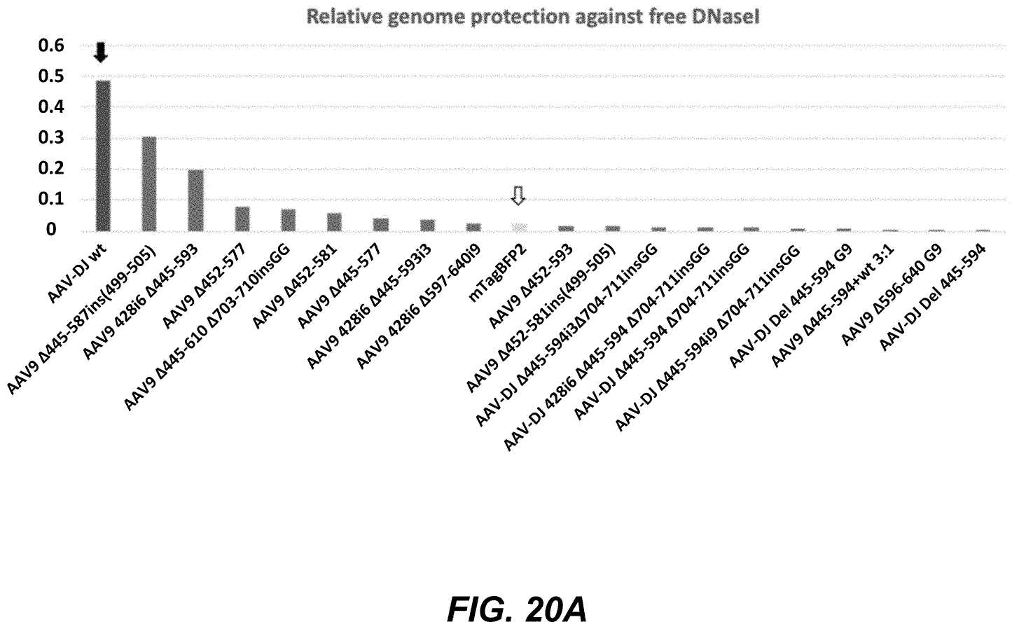

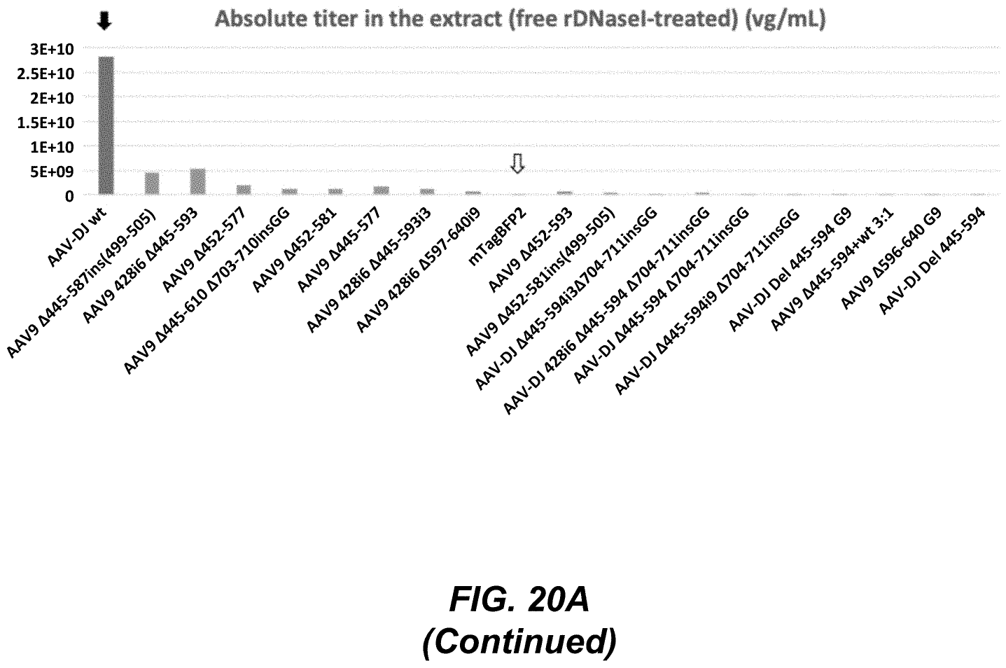

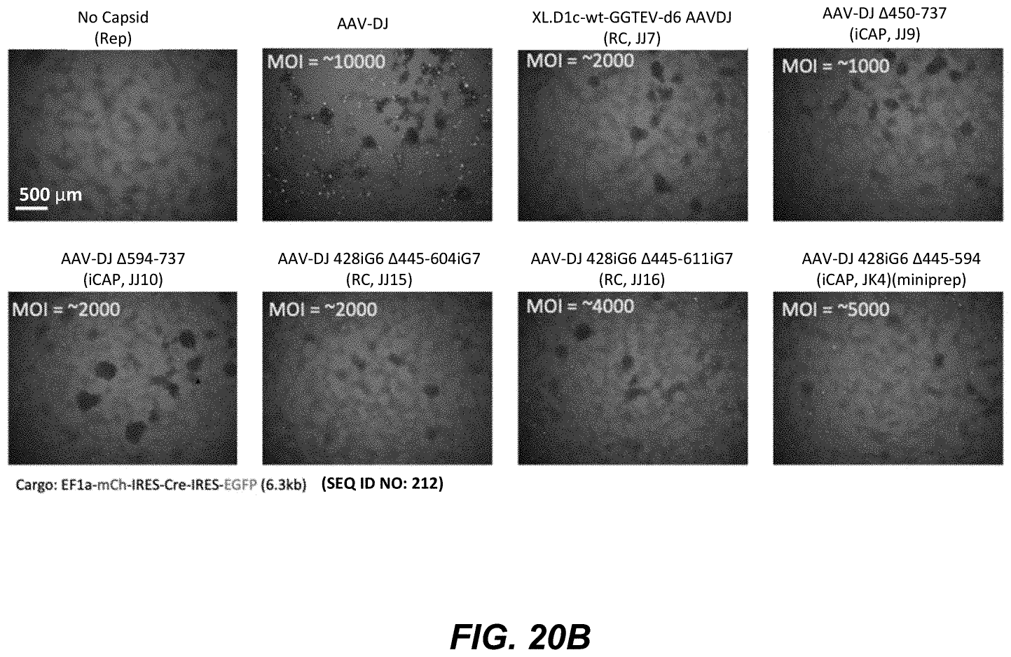

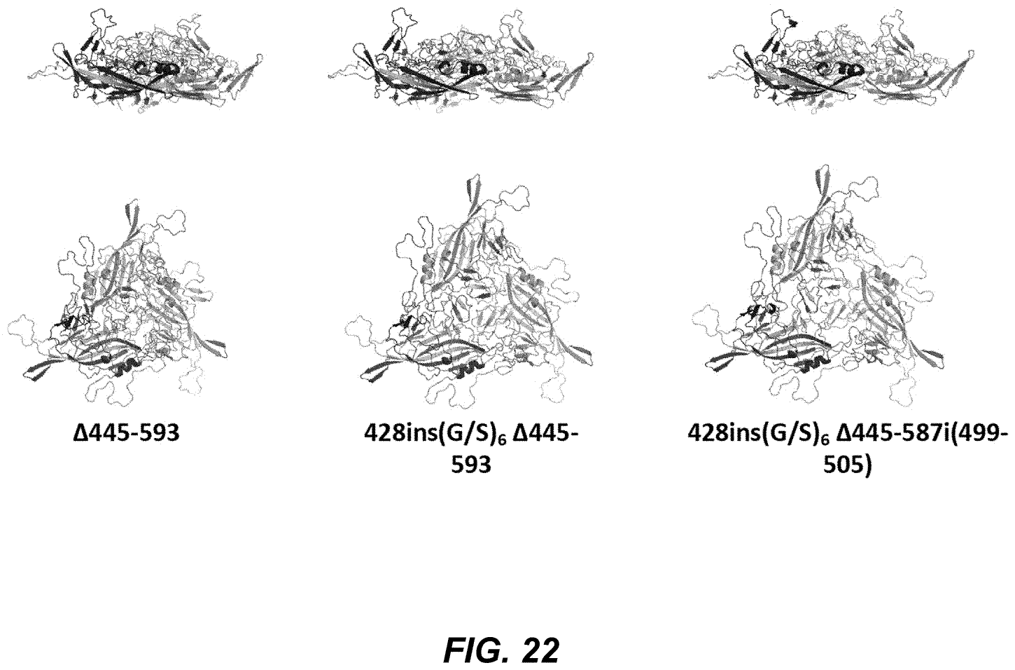

As it was previously found that extensions in the “hinge” linking the core block and a 3-fold interaction arm, or the “428 hinge”, could increase infectious titer in C-terminal truncated AAV capsids (FIG. 11A-FIG. 11D), similar designs with AAV-DJ Δ445-594 designs were tested. The results confirmed that they also make capsids with similar morphologies (FIG. 20A-FIG. 20B). AlphaFold2 was used to predict the trimer structures of AAV variants with deletions in the spike region and variants with both deletions in the spike region and insertions after the 428th residue. The results showed that the variants with hinge extension showed flatter and more regular structures with less steric clashing (FIG. 22).

All sequences tested in this Example 2 are listed below in Table 7:

TABLE 7
EXEMPLARY PEPTIDE SEQUENCES TESTED

| | Name | | | | |
|---|---|---|---|---|---|
| SEQ | (default | | | | Peptide sequence (starting |
| ID | backbone: | Other | Design | Observed | with the 1st residue |
| NO | AAV9) | names | rationale | phenotypes | in AAV9 or AAV-DJ VP1) |
| 74 | AAV9 Δ445- | N/A | 3-fold spike | Forms genome- | MAADGYLPDWLEDNLSEGIREWWALKP |
| | 610 ins(G/S)9 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLGGGGSGG |
| | | | | | GGDVYLQGPIWAKIPHTDGNFHPSPLMG |
| | | | | | GFGMKHPPPQILIKNTPVPADPPTAFNKD |
| | | | | | KLNSFITQYSTGQVSVEIEWELQKENSKR |
| | | | | | WNPEIQYTSNYYKSNNVEFAVNTEGVYS |
| | | | | | EPRPIGTRYLTRNL |
| 75 | AAV9 Δ445- | N/A | 3-fold spike | Forms genome- | MAADGYLPDWLEDNLSEGIREWWALKP |
| | 610 ins(G/S)7 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLGGGSGGG |
| | | | | | DVYLQGPIWAKIPHTDGNFHPSPLMGGFG |
| | | | | | MKHPPPQILIKNTPVPADPPTAFNKDKLNS |
| | | | | | FITQYSTGQVSVEIEWELQKENSKRWNPE |
| | | | | | IQYTSNYYKSNNVEFAVNTEGVYSEPRPI |
| | | | | | GTRYLTRNL |
| 76 | AAV9 Δ445- | N/A | 3-fold spike | Forms genome- | MAADGYLPDWLEDNLSEGIREWWALKP |
| | 603 ins(G/S)7 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLGGGSGGG |
| | | | | | GMVWQDRDVYLQGPIWAKIPHTDGNFHP |
| | | | | | SPLMGGFGMKHPPPQILIKNTPVPADPPTA |
| | | | | | FNKDKLNSFITQYSTGQVSVEIEWELQKE |
| | | | | | NSKRWNPEIQYTSNYYKSNNVEFAVNTE |
| | | | | | GVYSEPRPIGTRYLTRNL |
| 77 | AAV-DJ | N/A | 3-fold spike | Forms genome- | MAADGYLPDWLEDTLSEGIRQWWKLKP |
| | Δ445-611 | | region | protecting | GPPPPKPAERHKDDSRGLVLPGYKYLGPF |
| | ins(G/S)9 | | trimming | spherical capsids. | NGLDKGEPVNEADAAALEHDKAYDRQL |
| | | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGGGSGGGG |
| | | | | | DVYLQGPIWAKIPHTDGHFHPSPLMGGFG |
| | | | | | LKHPPPQILIKNTPVPADPPTTFNQSKLNSF |
| | | | | | ITQYSTGQVSVEIEWELQKENSKRWNPEI |
| | | | | | QYTSNYYKSTSVDFAVNTEGVYSEPRPIG |
| | | | | | TRYLTRNL |
| 78 | AAV-DJ | N/A | 3-fold spike | Forms genome- | MAADGYLPDWLEDTLSEGIRQWWKLKP |
| | Δ445-611 | | region | protecting | GPPPPKPAERHKDDSRGLVLPGYKYLGPF |
| | ins(G/S)9 | | trimming | spherical capsids. | NGLDKGEPVNEADAAALEHDKAYDRQL |
| | Δ707-737 | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGGGSGGGG |
| | | | | | DVYLQGPIWAKIPHTDGHFHPSPLMGGFG |
| | | | | | LKHPPPQILIKNTPVPADPPTTFNQSKLNSF |
| | | | | | ITQYSTGQVSVEIEWELQKENSKRWNPEI |
| | | | | | QYTSNY |
| 79 | AAV-DJ | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | | | trimming | spherical capsids. | NGLDKGEPVNEADAAALEHDKAYDRQL |
| | | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLADVNTQGVL |
| | | | | | PGMVWQDRDVYLQGPIWAKIPHTDGHFH |
| | | | | | PSPLMGGFGLKHPPPQILIKNTPVPADPPT |
| | | | | | TFNQSKLNSFITQYSTGQVSVEIEWELQKE |
| | | | | | NSKRWNPEIQYTSNYYKSTSVDFAVNTE |
| | | | | | GVYSEPRPIGTRYLTRNL |
| 80 | AAV-DJ | N/A | 3-fold spike | Forms genome- | MAADGYLPDWLEDTLSEGIRQWWKLKP |
| | 428ins(G/S)6 | | region | protecting | GPPPPKPAERHKDDSRGLVLPGYKYLGPF |
| | Δ445-594 | | trimming + | spherical capsids. | NGLDKGEPVNEADAAALEHDKAYDRQL |
| | | | 428 hinge | | DSGDNPYLKYNHADAEFQERLKEDTSFG |
| | | | region | | GNLGRAVFQAKKRLLEPLGLVEEAAKTA |
| | | | extension | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLADV |
| | | | | | NTQGVLPGMVWQDRDVYLQGPIWAKIP |
| | | | | | HTDGHFHPSPLMGGFGLKHPPPQILIKNTP |
| | | | | | VPADPPTTFNQSKLNSFITQYSTGQVSVEI |
| | | | | | EWELQKENSKRWNPEIQYTSNYYKSTSV |
| | | | | | DFAVNTEGVYSEPRPIGTRYLTRNL |
| 81 | AAV-DJ | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | ins(G/S)3 | | trimming | spherical capsids. | NGLDKGEPVNEADAAALEHDKAYDRQL |
| | | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGSADVNTQ |
| | | | | | GVLPGMVWQDRDVYLQGPIWAKIPHTD |
| | | | | | GHFHPSPLMGGFGLKHPPPQILIKNTPVPA |
| | | | | | DPPTTFNQSKLNSFITQYSTGQVSVEIEWE |
| | | | | | LQKENSKRWNPEIQYTSNYYKSTSVDFA |
| | | | | | VNTEGVYSEPRPIGTRYLTRNL |
| 82 | AAV-DJ | N/A | 3-fold spike | Forms genome- | MAADGYLPDWLEDTLSEGIRQWWKLKP |
| | 428ins(G/S)6, | | region | protecting | GPPPPKPAERHKDDSRGLVLPGYKYLGPF |
| | Δ445-594 | | trimming + | spherical capsids. | NGLDKGEPVNEADAAALEHDKAYDRQL |
| | ins(G/S)3 | | 428 hinge | | DSGDNPYLKYNHADAEFQERLKEDTSFG |
| | | | region | | GNLGRAVFQAKKRLLEPLGLVEEAAKTA |
| | | | extension | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLGGS |
| | | | | | ADVNTQGVLPGMVWQDRDVYLQGPIWA |
| | | | | | KIPHTDGHFHPSPLMGGFGLKHPPPQILIK |
| | | | | | NTPVPADPPTTFNQSKLNSFITQYSTGQVS |
| | | | | | VEIEWELQKENSKRWNPEIQYTSNYYKST |
| | | | | | SVDFAVNTEGVYSEPRPIGTRYLTRNL |
| 83 | AAV-DJ | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | ins(G/S)6 | | trimming + | spherical capsids. | NGLDKGEPVNEADAAALEHDKAYDRQL |
| | | | 428 hinge | | DSGDNPYLKYNHADAEFQERLKEDTSFG |
| | | | region | | GNLGRAVFQAKKRLLEPLGLVEEAAKTA |
| | | | extension | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGSGGSADV |
| | | | | | NTQGVLPGMVWQDRDVYLQGPIWAKIP |
| | | | | | HTDGHFHPSPLMGGFGLKHPPPQILIKNTP |
| | | | | | VPADPPTTFNQSKLNSFITQYSTGQVSVEI |
| | | | | | EWELQKENSKRWNPEIQYTSNYYKSTSV |
| | | | | | DFAVNTEGVYSEPRPIGTRYLTRNL |
| 84 | AAV-DJ | N/A | 3-fold spike | Forms genome- | MAADGYLPDWLEDTLSEGIRQWWKLKP |
| | 428ins(G/S)6, | | region | protecting | GPPPPKPAERHKDDSRGLVLPGYKYLGPF |
| | Δ445-594 | | trimming + | spherical capsids. | NGLDKGEPVNEADAAALEHDKAYDRQL |
| | ins(G/S)6 | | 428 hinge | | DSGDNPYLKYNHADAEFQERLKEDTSFG |
| | | | region | | GNLGRAVFQAKKRLLEPLGLVEEAAKTA |
| | | | extension | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLGGS |
| | | | | | GGSADVNTQGVLPGMVWQDRDVYLQGP |
| | | | | | IWAKIPHTDGHFHPSPLMGGFGLKHPPPQI |
| | | | | | LIKNTPVPADPPTTFNQSKLNSFITQYSTG |
| | | | | | QVSVEIEWELQKENSKRWNPEIQYTSNYY |
| | | | | | KSTSVDFAVNTEGVYSEPRPIGTRYLTRN |
| | | | | | L |
| 85 | AAV-DJ | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | ins(G/S)9 | | trimming | spherical capsids. | NGLDKGEPVNEADAAALEHDKAYDRQL |
| | | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGSGGSGGS |
| | | | | | ADVNTQGVLPGMVWQDRDVYLQGPIWA |
| | | | | | KIPHTDGHFHPSPLMGGFGLKHPPPQILIK |
| | | | | | NTPVPADPPTTFNQSKLNSFITQYSTGQVS |
| | | | | | VEIEWELQKENSKRWNPEIQYTSNYYKST |
| | | | | | SVDFAVNTEGVYSEPRPIGTRYLTRNL |
| 86 | AAV-DJ | N/A | 3-fold spike | Forms genome- | MAADGYLPDWLEDTLSEGIRQWWKLKP |
| | 428ins(G/S)6, | | region | protecting | GPPPPKPAERHKDDSRGLVLPGYKYLGPF |
| | Δ445-594 | | trimming + | spherical capsids. | NGLDKGEPVNEADAAALEHDKAYDRQL |
| | ins(G/S)9 | | 428 hinge | | DSGDNPYLKYNHADAEFQERLKEDTSFG |
| | | | region | | GNLGRAVFQAKKRLLEPLGLVEEAAKTA |
| | | | extension | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLGGS |
| | | | | | GGSGGSADVNTQGVLPGMVWQDRDVYL |
| | | | | | QGPIWAKIPHTDGHFHPSPLMGGFGLKHP |
| | | | | | PPQILIKNTPVPADPPTTFNQSKLNSFITQY |
| | | | | | STGQVSVEIEWELQKENSKRWNPEIQYTS |
| | | | | | NYYKSTSVDFAVNTEGVYSEPRPIGTRYL |
| | | | | | TRNL |
| 87 | AAV9 Δ445- | N/A | 3-fold spike | Forms genome- | MAADGYLPDWLEDNLSEGIREWWALKP |
| | 587ins(499- | | region | protecting | GAPQPKANQQHQDNARGLVLPGYKYLG |
| | 505) | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLEFAWPGQ |
| | | | | | QAQAQTGWVQNQGILPGMVWQDRDVYL |
| | | | | | QGPIWAKIPHTDGNFHPSPLMGGFGMKH |
| | | | | | PPPQILIKNTPVPADPPTAFNKDKLNSFITQ |
| | | | | | YSTGQVSVEIEWELQKENSKRWNPEIQYT |
| | | | | | SNYYKSNNVEFAVNTEGVYSEPRPIGTRY |
| | | | | | LTRNL |
| 88 | AAV9 428i6 | N/A | 3-fold spike | Forms genome- | MAADGYLPDWLEDNLSEGIREWWALKP |
| | Δ445-593 | | region | protecting | GAPQPKANQQHQDNARGLVLPGYKYLG |
| | | | trimming + | spherical capsids. | PGNGLDKGEPVNAADAAALEHDKAYDQ |
| | | | 428 hinge | | QLKAGDNPYLKYNHADAEFQERLKEDTS |
| | | | region | | FGGNLGRAVFQAKKRLLEPLGLVEEAAK |
| | | | extension | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSGGSGGSQSLDRLMNPLIDQYLG |
| | | | | | WVQNQGILPGMVWQDRDVYLQGPIWAK |
| | | | | | IPHTDGNFHPSPLMGGFGMKHPPPQILIKN |
| | | | | | TPVPADPPTAFNKDKLNSFITQYSTGQVS |
| | | | | | VEIEWELQKENSKRWNPEIQYTSNYYKSN |
| | | | | | NVEFAVNTEGVYSEPRPIGTRYLTRNL |
| 89 | AAV9 Δ452- | N/A | 3-fold spike | Forms genome- | MAADGYLPDWLEDNLSEGIREWWALKP |
| | 577 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIG |
| | | | | | QVATNHQSAQAQAQTGWVQNQGILPGM |
| | | | | | VWQDRDVYLQGPIWAKIPHTDGNFHPSP |
| | | | | | LMGGFGMKHPPPQILIKNTPVPADPPTAF |
| | | | | | NKDKLNSFITQYSTGQVSVEIEWELQKEN |
| | | | | | SKRWNPEIQYTSNYYKSNNVEFAVNTEG |
| | | | | | VYSEPRPIGTRYLTRNL |
| 90 | AAV9 Δ445- | N/A | 3-fold spike | Forms genome- | MAADGYLPDWLEDNLSEGIREWWALKP |
| | 610 Δ703- | | region | protecting | GAPQPKANQQHQDNARGLVLPGYKYLG |
| | 710insGG | | trimming + 2- | spherical capsids. | PGNGLDKGEPVNAADAAALEHDKAYDQ |
| | | | fold sealer | | QLKAGDNPYLKYNHADAEFQERLKEDTS |
| | | | block | | FGGNLGRAVFQAKKRLLEPLGLVEEAAK |
| | | | trimming | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLDVYLQGPI |
| | | | | | WAKIPHTDGNFHPSPLMGGFGMKHPPPQI |
| | | | | | LIKNTPVPADPPTAFNKDKLNSFITQYSTG |
| | | | | | QVSVEIEWELQKENSKRWNPEIQYTGGVE |
| | | | | | FAVNTEGVYSEPRPIGTRYLTRNL |
| 91 | AAV9 Δ452- | N/A | 3-fold spike | Forms genome- | MAADGYLPDWLEDNLSEGIREWWALKP |
| | 581 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIT |
| | | | | | NHQSAQAQAQTGWVQNQGILPGMVWQ |
| | | | | | DRDVYLQGPIWAKIPHTDGNFHPSPLMG |
| | | | | | GFGMKHPPPQILIKNTPVPADPPTAFNKD |
| | | | | | KLNSFITQYSTGQVSVEIEWELQKENSKR |
| | | | | | WNPEIQYTSNYYKSNNVEFAVNTEGVYS |
| | | | | | EPRPIGTRYLTRNL |
| 92 | AAV9 Δ445- | N/A | 3-fold spike | Forms genome- | MAADGYLPDWLEDNLSEGIREWWALKP |
| | 577 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLGQVATNH |
| | | | | | QSAQAQAQTGWVQNQGILPGMVWQDRD |
| | | | | | VYLQGPIWAKIPHTDGNFHPSPLMGGFG |
| | | | | | MKHPPPQILIKNTPVPADPPTAFNKDKLNS |
| | | | | | FITQYSTGQVSVEIEWELQKENSKRWNPE |
| | | | | | IQYTSNYYKSNNVEFAVNTEGVYSEPRPI |
| | | | | | GTRYLTRNL |
| 93 | AAV9 428i6 | N/A | 3-fold spike | Forms genome- | MAADGYLPDWLEDNLSEGIREWWALKP |
| | Δ445-593i3 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSGGSGGSQSLDRLMNPLIDQYLGG |
| | | | | | SGWVQNQGILPGMVWQDRDVYLQGPIW |
| | | | | | AKIPHTDGNFHPSPLMGGFGMKHPPPQILI |
| | | | | | KNTPVPADPPTAFNKDKLNSFITQYSTGQ |
| | | | | | VSVEIEWELQKENSKRWNPEIQYTSNYYK |
| | | | | | SNNVEFAVNTEGVYSEPRPIGTRYLTRNL |
| 94 | AAV9 428i6 | N/A | 3-fold spike | Forms genome- | MAADGYLPDWLEDNLSEGIREWWALKP |
| | Δ597-640i9 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSGGSGGSQSLDRLMNPLIDQYLYY |
| | | | | | LSKTINGSGQNQQTLKFSVAGPSNMAVQ |
| | | | | | GRNYIPGPSYRQQRVSTTVTQNNNSEFA |
| | | | | | WPGASSWALNGRNSLMNPGPAMASHKE |
| | | | | | GEDRFFPLSGSLIFGKQGTGRDNVDADKV |
| | | | | | MITNEEEIKTTNPVATESYGQVATNHQSA |
| | | | | | QAQAQTGWVGGSGGSGGSKHPPPQILIK |
| | | | | | NTPVPADPPTAFNKDKLNSFITQYSTGQV |
| | | | | | SVEIEWELQKENSKRWNPEIQYTSNYYKS |
| | | | | | NNVEFAVNTEGVYSEPRPIGTRYLTRNL |
| 95 | AAV9 Δ452- | N/A | 3-fold spike | Forms genome- | MAADGYLPDWLEDNLSEGIREWWALKP |
| | 593 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIG |
| | | | | | WVQNQGILPGMVWQDRDVYLQGPIWAK |
| | | | | | IPHTDGNFHPSPLMGGFGMKHPPPQILIKN |
| | | | | | TPVPADPPTAFNKDKLNSFITQYSTGQVS |
| | | | | | VEIEWELQKENSKRWNPEIQYTSNYYKSN |
| | | | | | NVEFAVNTEGVYSEPRPIGTRYLTRNL |
| 96 | AAV9 Δ452- | N/A | 3-fold spike | Forms genome- | MAADGYLPDWLEDNLSEGIREWWALKP |
| | 581ins(499- | | region | protecting | GAPQPKANQQHQDNARGLVLPGYKYLG |
| | 505) | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIEF |
| | | | | | AWPGQTNHQSAQAQAQTGWVQNQGILP |
| | | | | | GMVWQDRDVYLQGPIWAKIPHTDGNFHP |
| | | | | | SPLMGGFGMKHPPPQILIKNTPVPADPPTA |
| | | | | | FNKDKLNSFITQYSTGQVSVEIEWELQKE |
| | | | | | NSKRWNPEIQYTSNYYKSNNVEFAVNTE |
| | | | | | GVYSEPRPIGTRYLTRNL |
| 97 | AAV-DJ | N/A | 3-fold spike | Forms genome- | MAADGYLPDWLEDTLSEGIRQWWKLKP |
| | Δ445-594i3 | | region | protecting | GPPPPKPAERHKDDSRGLVLPGYKYLGPF |
| | Δ704- | | trimming + 2- | spherical capsids. | NGLDKGEPVNEADAAALEHDKAYDRQL |
| | 711insGG | | fold sealer | | DSGDNPYLKYNHADAEFQERLKEDTSFG |
| | | | block | | GNLGRAVFQAKKRLLEPLGLVEEAAKTA |
| | | | trimming | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGSADVNTQ |
| | | | | | GVLPGMVWQDRDVYLQGPIWAKIPHTD |
| | | | | | GHFHPSPLMGGFGLKHPPPQILIKNTPVPA |
| | | | | | DPPTTFNQSKLNSFITQYSTGQVSVEIEWE |
| | | | | | LQKENSKRWNPEIQYTGGVDFAVNTEGV |
| | | | | | YSEPRPIGTRYLTRNL |
| 98 | AAV-DJ 428i6 | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | Δ704- | | trimming | spherical capsids. | NGLDKGEPVNEADAAALEHDKAYDRQL |
| | 711insGG | | | | DSGDNPYLKYNHADAEFQERLKEDTSFG |
| | | | | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLADV |
| | | | | | NTQGVLPGMVWQDRDVYLQGPIWAKIP |
| | | | | | HTDGHFHPSPLMGGFGLKHPPPQILIKNTP |
| | | | | | VPADPPTTFNQSKLNSFITQYSTGQVSVEI |
| | | | | | EWELQKENSKRWNPEIQYTGGVDFAVNT |
| | | | | | EGVYSEPRPIGTRYLTRNL |
| 99 | AAV-DJ | N/A | 3-fold spike | Forms genome- | MAADGYLPDWLEDTLSEGIRQWWKLKP |
| | Δ445-594 | | region | protecting | GPPPPKPAERHKDDSRGLVLPGYKYLGPF |
| | Δ704-711GG | | trimming + 2- | spherical capsids. | NGLDKGEPVNEADAAALEHDKAYDRQL |
| | | | fold sealer | | DSGDNPYLKYNHADAEFQERLKEDTSFG |
| | | | block | | GNLGRAVFQAKKRLLEPLGLVEEAAKTA |
| | | | trimming | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLADVNTQGVL |
| | | | | | PGMVWQDRDVYLQGPIWAKIPHTDGHFH |
| | | | | | PSPLMGGFGLKHPPPQILIKNTPVPADPPT |
| | | | | | TFNQSKLNSFITQYSTGQVSVEIEWELQKE |
| | | | | | NSKRWNPEIQYTGGVDFAVNTEGVYSEP |
| | | | | | RPIGTRYLTRNL |
| 100 | AAV-DJ | N/A | 3-fold spike | Forms genome- | MAADGYLPDWLEDTLSEGIRQWWKLKP |
| | Δ445-594i9 | | region | protecting | GPPPPKPAERHKDDSRGLVLPGYKYLGPF |
| | Δ704-711GG | | trimming + 2- | spherical capsids. | NGLDKGEPVNEADAAALEHDKAYDRQL |
| | | | fold sealer | | DSGDNPYLKYNHADAEFQERLKEDTSFG |
| | | | block | | GNLGRAVFQAKKRLLEPLGLVEEAAKTA |
| | | | trimming | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSQSLDRLMNPLIDQYLGGSGGSGGS |
| | | | | | ADVNTQGVLPGMVWQDRDVYLQGPIWA |
| | | | | | KIPHTDGHFHPSPLMGGFGLKHPPPQILIK |
| | | | | | NTPVPADPPTTFNQSKLNSFITQYSTGQVS |
| | | | | | VEIEWELQKENSKRWNPEIQYTGGVDFA |
| | | | | | VNTEGVYSEPRPIGTRYLTRNL |
| 101 | AAV9 Δ596- | N/A | 3-fold spike | Forms genome- | MAADGYLPDWLEDNLSEGIREWWALKP |
| | 640 G9 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIN |
| | | | | | GSGQNQQTLKFSVAGPSNMAVQGRNYIP |
| | | | | | GPSYRQQRVSTTVTQNNNSEFAWPGASS |
| | | | | | WALNGRNSLMNPGPAMASHKEGEDRFFP |
| | | | | | LSGSLIFGKQGTGRDNVDADKVMITNEEE |
| | | | | | IKTTNPVATESYGQVATNHQSAQAQAQT |
| | | | | | GWGGSGGSGGSKHPPPQILIKNTPVPADP |
| | | | | | PTAFNKDKLNSFITQYSTGQVSVEIEWEL |
| | | | | | QKENSKRWNPEIQYTSNYYKSNNVEFAV |
| | | | | | NTEGVYSEPRPIGTRYLTRNL |
| 102 | AAV-DJ | N/A | 3-fold spike | Forms genome- | MAADGYLPDWLEDTLSEGIRQWWKLKP |
| | 428iG6 Δ445- | | region | protecting | GPPPPKPAERHKDDSRGLVLPGYKYLGPF |
| | 604iG7 | | trimming + | spherical capsids. | NGLDKGEPVNEADAAALEHDKAYDRQL |
| | | | 428 hinge | | DSGDNPYLKYNHADAEFQERLKEDTSFG |
| | | | extension | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLGGG |
| | | | | | SGGGGMVWQDRDVYLQGPIWAKIPHTD |
| | | | | | GHFHPSPLMGGFGLKHPPPQILIKNTPVPA |
| | | | | | DPPTTFNQSKLNSFITQYSTGQVSVEIEWE |
| | | | | | LQKENSKRWNPEIQYTSNYYKSTSVDFA |
| | | | | | VNTEGVYSEPRPIGTRYLTRNL |
| 103 | AAV-DJ | N/A | 3-fold spike | Forms genome- | MAADGYLPDWLEDTLSEGIRQWWKLKP |
| | 428iG6 Δ445- | | region | protecting | GPPPPKPAERHKDDSRGLVLPGYKYLGPF |
| | 61HG7 | | trimming + | spherical capsids. | NGLDKGEPVNEADAAALEHDKAYDRQL |
| | | | 428 hinge | | DSGDNPYLKYNHADAEFQERLKEDTSFG |
| | | | extension | | GNLGRAVFQAKKRLLEPLGLVEEAAKTA |
| | | | | | PGKKRPVEHSPVEPDSSSGTGKAGQQPAR |
| | | | | | KRLNFGQTGDADSVPDPQPIGEPPAAPSG |
| | | | | | VGSLTMAAGGGAPMADNNEGADGVGNS |
| | | | | | SGNWHCDSTWMGDRVITTSTRTWALPTY |
| | | | | | NNHLYKQISNSTSGGSSNDNAYFGYSTPW |
| | | | | | GYFDFNRFHCHFSPRDWQRLINNNWGFR |
| | | | | | PKRLSFKLFNIQVKEVTQNEGTKTIANNL |
| | | | | | TSTIQVFTDSEYQLPYVLGSAHQGCLPPFP |
| | | | | | ADVFMIPQYGYLTLNNGSQAVGRSSFYC |
| | | | | | LEYFPSQMLRTGNNFQFTYTFEDVPFHSS |
| | | | | | YAHSGGSGGSQSLDRLMNPLIDQYLGGG |
| | | | | | SGGGDVYLQGPIWAKIPHTDGHFHPSPLM |
| | | | | | GGFGLKHPPPQILIKNTPVPADPPTTFNQS |
| | | | | | KLNSFITQYSTGQVSVEIEWELQKENSKR |
| | | | | | WNPEIQYTSNYYKSTSVDFAVNTEGVYSE |
| | | | | | PRPIGTRYLTRNL |
| 104 | AAV9 Δ428- | N/A | 3-fold spike | Forms genome- | MAADGYLPDWLEDNLSEGIREWWALKP |
| | 640GS9 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAGGGGSGGGGKHPPPQILIKNTPVPA |
| | | | | | DPPTAFNKDKLNSFITQYSTGQVSVEIEW |
| | | | | | ELQKENSKRWNPEIQYTSNYYKSNNVEF |
| | | | | | AVNTEGVYSEPRPIGTRYLTRNL |
| 105 | AAV9 Δ418- | N/A | 3-fold spike | Forms genome- | MAADGYLPDWLEDNLSEGIREWWALKP |
| | 640GS3 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFGSGKHP |
| | | | | | PPQILIKNTPVPADPPTAFNKDKLNSFITQ |
| | | | | | YSTGQVSVEIEWELQKENSKRWNPEIQYT |
| | | | | | SNYYKSNNVEFAVNTEGVYSEPRPIGTRY |
| | | | | | LTRNL |
| 106 | AAV9 Δ429- | N/A | 3-fold spike | Forms genome- | MAADGYLPDWLEDNLSEGIREWWALKP |
| | 607GS4 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHGGGSQDRDVYLQGPIWAKIPHTD |
| | | | | | GNFHPSPLMGGFGMKHPPPQILIKNTPVP |
| | | | | | ADPPTAFNKDKLNSFITQYSTGQVSVEIE |
| | | | | | WELQKENSKRWNPEIQYTSNYYKSNNVE |
| | | | | | FAVNTEGVYSEPRPIGTRYLTRNL |
| 107 | XL.Dc-AAV9 | N/A | Dimerized | Forms genome- | MAADGYLPDWLEDNLSEGIREWWALKP |
| | Δ712-736 | | capsid | protecting | GAPQPKANQQHQDNARGLVLPGYKYLG |
| | | | subunit | spherical capsids. | PGNGLDKGEPVNAADAAALEHDKAYDQ |
| | | | with 2-fold | | QLKAGDNPYLKYNHADAEFQERLKEDTS |
| | | | ″sealer″ | | FGGNLGRAVFQAKKRLLEPLGLVEEAAK |
| | | | region | | TAPGKKRPVEQSPQEPDSSAGIGKSGAQP |
| | | | trimming | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIN |
| | | | | | GSGQNQQTLKFSVAGPSNMAVQGRNYIP |
| | | | | | GPSYRQQRVSTTVTQNNNSEFAWPGASS |
| | | | | | WALNGRNSLMNPGPAMASHKEGEDRFFP |
| | | | | | LSGSLIFGKQGTGRDNVDADKVMITNEEE |
| | | | | | IKTTNPVATESYGQVATNHQSAQAQAQT |
| | | | | | GWVQNQGILPGMVWQDRDVYLQGPIWA |
| | | | | | KIPHTDGNFHPSPLMGGFGMKHPPPQILIK |
| | | | | | NTPVPADPPTAFNKDKLNSFITQYSTGQV |
| | | | | | SVEIEWELQKENSKRWNPEIQYTSNYYKS |
| | | | | | NNVEFAVNTEGVYSEPRPIGTRYLTRNLG |
| | | | | | GENLYFQGASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLYYLSKTIN |
| | | | | | GSGQNQQTLKFSVAGPSNMAVQGRNYIP |
| | | | | | GPSYRQQRVSTTVTQNNNSEFAWPGASS |
| | | | | | WALNGRNSLMNPGPAMASHKEGEDRFFP |
| | | | | | LSGSLIFGKQGTGRDNVDADKVMITNEEE |
| | | | | | IKTTNPVATESYGQVATNHQSAQAQAQT |
| | | | | | GWVQNQGILPGMVWQDRDVYLQGPIWA |
| | | | | | KIPHTDGNFHPSPLMGGFGMKHPPPQILIK |
| | | | | | NTPVPADPGSLNSFITQYSTGQVSVEIEWE |
| | | | | | LQKENSKRWNPEIQYTSNYYKSNNV |
| 108 | AAV9 Δ445- | N/A | 3-fold spike | Forms genome- | MAADGYLPDWLEDNLSEGIREWWALKP |
| | 593 | | region | protecting | GAPQPKANQQHQDNARGLVLPGYKYLG |
| | | | trimming | spherical capsids. | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | PFPADVFMIPQYGYLTLNDGSQAVGRSSF |
| | | | | | YCLEYFPSQMLRTGNNFQFSYEFENVPFH |
| | | | | | SSYAHSQSLDRLMNPLIDQYLGWVQNQG |
| | | | | | ILPGMVWQDRDVYLQGPIWAKIPHTDGN |
| | | | | | FHPSPLMGGFGMKHPPPQILIKNTPVPADP |
| | | | | | PTAFNKDKLNSFITQYSTGQVSVEIEWEL |
| | | | | | QKENSKRWNPEIQYTSNYYKSNNVEFAV |
| | | | | | NTEGVYSEPRPIGTRYLTRNL |
| 109 | Wild-type | | | | MAADGYLPDWLEDNLSEGIREWWALKP |
| | AAV9 | | | | GAPQPKANQQHQDNARGLVLPGYKYLG |
| | | | | | PGNGLDKGEPVNAADAAALEHDKAYDQ |
| | | | | | QLKAGDNPYLKYNHADAEFQERLKEDTS |
| | | | | | FGGNLGRAVFQAKKRLLEPLGLVEEAAK |
| | | | | | TAPGKKRPVEQSPQEPDSSAGIGKSGAQP |
| | | | | | AKKRLNFGQTGDTESVPDPQPIGEPPAAP |
| | | | | | SGVGSLTMASGGGAPVADNNEGADGVG |
| | | | | | SSSGNWHCDSQWLGDRVITTSTRTWALP |
| | | | | | TYNNHLYKQISNSTSGGSSNDNAYFGYST |
| | | | | | PWGYFDFNRFHCHFSPRDWQRLINNNWG |
| | | | | | FRPKRLNFKLFNIQVKEVTDNNGVKTIAN |
| | | | | | NLTSTVQVFTDSDYQLPYVLGSAHEGCLP |
| | | | | | YCLEYFPSQMLRTGNNFQFSYEFENVPFH |
| | | | | | SSYAHSQSLDRLMNPLIDQYLYYLSKTIN |
| | | | | | GSGQNQQTLKFSVAGPSNMAVQGRNYIP |
| | | | | | GPSYRQQRVSTTVTQNNNSEFAWPGASS |
| | | | | | WALNGRNSLMNPGPAMASHKEGEDRFFP |
| | | | | | LSGSLIFGKQGTGRDNVDADKVMITNEEE |
| | | | | | IKTTNPVATESYGQVATNHQSAQAQAQT |
| | | | | | GWVQNQGILPGMVWQDRDVYLQGPIWA |
| | | | | | KIPHTDGNFHPSPLMGGFGMKHPPPQILIK |
| | | | | | NTPVPADPPTAFNKDKLNSFITQYSTGQV |
| | | | | | SVEIEWELQKENSKRWNPEIQYTSNYYKS |
| | | | | | NNVEFAVNTEGVYSEPRPIGTRYLTRNL |

The starting residue of all sequences is M1.

Spike-Trimmed Capsid Variants Retain Partial Genome Protection and Transduction Capability, Allowing for Future Evolution

Whether these capsid structures can protect the cargo genome from nuclease digestion was tested. Lysates of producer cells of different variants were treated with either free recombinant DNAse I or immobilized recombinant DNAse I, and the numbers of genome copies in treated samples were titered with qPCR. Although none of the spike-trimmed variants is perfectly resistant to free DNase I as wild-type AAV-DJ, a few of the variants did show partial protection of the genomes that are much higher than the no-capsid controls (FIG. 20A). Some of the variants also showed the capability of transducing cultured HEK293T cells (FIG. 20B). Based on our systematic truncations of an AAV capsid protein, we found that the size-determining factor for AAV capsid assembly appears to reside in the 3-fold interacting spike region and the 2-fold interacting sealer block. When truncated, they may produce capsids with increased sizes. Most of the size-expanded capsids can package and protect oversized genomes. Although some of such deletions cause decreased homogeneity and infectivity, these properties may be improved by further engineering.

Although AAV capsid variants with specific deletions within the spike region have produced size-expanded capsids with decent titers, they still suffer from relatively low transduction efficiency. Without being bound by any particular theory, in some embodiments, one reason is that the newly exposed capsid protein surfaces are not optimized for solubility and new types of inter-subunit interactions. In some embodiments, the loss in transduction efficiency is because of the loss of residues in the 3-fold spike that are necessary for virus-receptor interactions. However, for a similar reason, the truncated capsid variants can be less immunogenic in vivo, which, can be beneficial for their applications as gene delivery vectors.

In one embodiment, well-established directed evolution technologies can be used to optimize the new surface/interface residues for improved capsid stability, packaging efficiency, and transduction efficiency.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A variant adeno-associated virus (AAV) capsid protein, wherein (a) fifty or more of amino acid residues functionally equivalent to amino acids 452 to 577 of the AAV9 VP1 protein (SEQ ID NO: 109) have been deleted or substituted, (b) twenty or more of amino acid residues functionally equivalent to amino acids 597 to 640 of the AAV9 VP1 protein (SEQ ID NO: 109) have been deleted or substituted, (c) five or more of amino acid residues functionally equivalent to amino acids 656 to 669 of the AAV9 VP1 protein (SEQ ID NO: 109) have been deleted or substituted, (d) five or more of amino acid residues functionally equivalent to amino acids 692 to 736 of the AAV9 VP1 protein (SEQ ID NO: 109) have been deleted or substituted, or a combination thereof.

2. (canceled)

3. The variant AAV capsid protein of claim 1, comprising a deletion or substitution of the amino acid residues functionally equivalent to amino acids 452 to 577 of the AAV9 VP1 protein (SEQ ID NO: 109).

4. The variant AAV capsid protein of claim 1, comprising a deletion or substitution of the amino acid residues functionally equivalent to amino acids 433 to 640 of the AAV9 VP1 protein (SEQ ID NO: 109), amino acids 445 to 577 of the AAV9 VP1 protein (SEQ ID NO: 109), amino acids 445 to 587 of the AAV9 VP1 protein (SEQ ID NO: 109), amino acids 445 to 593 of the AAV9 VP1 protein (SEQ ID NO: 109), amino acids 445 to 594 of the AAV9 VP1 protein (SEQ ID NO: 109), amino acids 445 to 603 of the AAV9 VP1 protein (SEQ ID NO: 109), amino acids 445 to 604 of the AAV9 VP1 protein (SEQ ID NO: 109), amino acids 445 to 610 of the AAV9 VP1 protein (SEQ ID NO: 109), amino acids 445 to 611 of the AAV9 VP1 protein (SEQ ID NO: 109), amino acids 445 to 640 of the AAV9 VP1 protein (SEQ ID NO: 109), amino acids 445 to 691 of the AAV9 VP1 protein (SEQ ID NO: 109), amino acids 452 to 577 of the AAV9 VP1 protein (SEQ ID NO: 109), amino acids 452 to 581 of the AAV9 VP1 protein (SEQ ID NO: 109), amino acids 452 to 593 of the AAV9 VP1 protein (SEQ ID NO: 109), amino acids 452 to 599 of the AAV9 VP1 protein (SEQ ID NO: 109), amino acids 429 to 607 of the AAV9 VP1 protein (SEQ ID NO: 109), amino acids 428 to 640 of the AAV9 VP1 protein (SEQ ID NO: 109), or amino acids 418 to 640 of the AAV9 VP1 protein (SEQ ID NO: 109).

5. The variant AAV capsid protein of claim 1, comprising a deletion or substitution of the amino acid residues functionally equivalent to amino acids 593 to 640 of the AAV9 VP1 protein (SEQ ID NO: 109), amino acids 594 to 640 of the AAV9 VP1 protein (SEQ ID NO: 109), or amino acids 596 to 640 of the AAV9 VP1 protein (SEQ ID NO: 109).

6. The variant AAV capsid protein of claim 1, comprising a deletion or substitution of the amino acid residues functionally equivalent to amino acids 704 to 711 of the AAV9 VP1 protein (SEQ ID NO: 109), amino acids 704 to 727 of the AAV9 VP1 protein (SEQ ID NO: 109), amino acids 704 to 736 of the AAV9 VP1 protein (SEQ ID NO: 109), amino acids 706 to 736 of the AAV9 VP1 protein (SEQ ID NO: 109), amino acids 712 to 736 of the AAV9 VP1 protein (SEQ ID NO: 109), amino acids 693 to 736 of the AAV9 VP1 protein (SEQ ID NO: 109), or amino acids 692 to 736 of the AAV9 VP1 protein (SEQ ID NO: 109).

7. The variant AAV capsid protein of claim 1, comprising a deletion or substitution of the amino acid residues functionally equivalent to amino acids 658 to 667 of the AAV9 VP1 protein (SEQ ID NO: 109), or amino acids 659 to 666 of the AAV9 VP1 protein (SEQ ID NO: 109).

8. The variant AAV capsid protein of claim 1, comprising a deletion or substitution of the amino acid residues functionally equivalent to amino acids 426 to 736 of the AAV9 VP1 protein (SEQ ID NO: 109), amino acids 444 to 736 of the AAV9 VP1 protein, amino acids 445 to 736 of the AAV9 VP1 protein (SEQ ID NO: 109), amino acids 452 to 736 of the AAV9 VP1 protein (SEQ ID NO: 109), or amino acids 450 to 736 of the AAV9 VP1 protein (SEQ ID NO: 109).

9. The variant AAV capsid protein of claim 1, wherein the substitution is by a peptide segment.

10. (canceled)

11. The variant AAV capsid protein of claim 9, wherein the peptide segment is a flexible peptide segment.

12. (canceled)

13. (canceled)

14. The variant AAV capsid protein of claim 1, wherein the variant AAV capsid protein comprises the amino acid residues functionally equivalent to Y426, A427, and H428 of the AAV9 VP1 protein (SEQ ID NO: 109).

15. (canceled)

16. (canceled)

17. (canceled)

18. (canceled)

19. (canceled)

20. (canceled)

21. A variant adeno-associated virus (AAV) capsid protein, wherein (a) fifty or more of the amino acid residues functionally equivalent to amino acids 452 to 581 of the AAV9 VP1 protein (SEQ ID NO: 109) have been deleted, and (b) the variant AAV capsid protein comprises five or more of the deleted amino acids in (a) in the C-terminus.

22. The variant AAV capsid protein of claim 21, wherein (a) the amino acid residues functionally equivalent to amino acids 417 to 640 of the AAV9 VP1 protein (SEQ ID NO: 109) have been substituted by a peptide segment of GGS and (b) the variant AAV capsid protein comprises the deleted amino acids 430 to 640 of the AAV9 VP1 protein (SEQ ID NO: 109) in (a) in the C-terminus.

23. The variant AAV capsid protein of claim 21, wherein (a) the amino acid residues functionally equivalent to amino acids 417 to 640 of the AAV9 VP1 protein (SEQ ID NO: 109) have been substituted by a peptide segment of GGSGGGS (SEQ ID NO: 122) and (b) the variant AAV capsid protein comprises the deleted amino acids 430 to 640 of the AAV9 VP1 protein (SEQ ID NO: 109) in (a) in the C-terminus.

24. A variant adeno-associated virus (AAV) capsid, comprising a variant AAV capsid protein of claim 1.

25. A variant adeno-associated virus (AAV) capsid, wherein the AAV capsid comprises a plurality of multimers each comprising two or more AAV capsid proteins, wherein at least one of the two or more AAV capsid proteins is a variant AAV capsid protein of claim 1.

26. The variant AAV capsid of claim 25, wherein two of the two or more AAV capsid proteins are connected by a linker.

27. (canceled)

28. The variant AAV capsid of claim 25, wherein the variant AAV capsid comprises two or more multimers that differ with respect to the capsid protein isoforms that compose the multimers.

29.-45. (canceled)

46. A recombinant AAV (rAAV), the rAAV comprising:
the variant AAV capsid of claim 24; and
a heterologous nucleic acid, wherein the heterologous nucleic acid comprises a polynucleotide encoding a payload, and wherein the payload comprises a payload RNA agent and/or a payload protein.

47.-81. (canceled)

82. A composition, comprising
the variant AAV capsid protein of claim 1; and
a pharmaceutically acceptable carrier.

83. (canceled)

84. (canceled)

85. (canceled)

86. (canceled)

87. A method of introducing a nucleic acid into a cell, comprising
contacting the cell with the composition of claim 82.

88.-103. (canceled)
